SEQUENCE LISTING (37 sequences)

SEQ ID NO: 1 - DNA, 20 bp, Artificial sequence
  synthetic sequence
  misc_binding (1)..(1): The nucleotide at position 1 comprises Cy3
cagtcagtcagtcagtcagt

SEQ ID NO: 2 - DNA, 20 bp, Artificial sequence
  synthetic sequence
  misc_binding (1)..(1): The nucleotide at position 1 comprises Cy5
cagtcagtcagtcagtcagt

SEQ ID NO: 3 - DNA, 20 bp, Artificial sequence
  synthetic sequence
  misc_binding (1)..(1): The nucleotide at position 1 comprises AlexaFluor488
ttcttcttcgttcttcttct

SEQ ID NO: 4 - DNA, 100 bp, Artificial sequence
  synthetic sequence
  misc_binding (1)..(1): The nucleotide at position 1 comprises a lipid
gtaacgatccagctgtcactttttttttttttttttttttttttttttttttttttttttttttttttttttttttttttttcttcttcgttcttcttct

SEQ ID NO: 5 - DNA, 20 bp, Artificial sequence
  synthetic sequence
  misc_binding (1)..(1): The nucleotide at position 1 comprises an azide
  misc_binding (20)..(20): The nucleotide at position 20 comprises Cy3
ctctctctctctctctggct

SEQ ID NO: 6 - DNA, 20 bp, Artificial sequence
  synthetic sequence
  misc_binding (1)..(1): The nucleotide at position 1 comprises Cy5
ctctctctctctctctggct

SEQ ID NO: 7 - DNA, 20 bp, Artificial sequence
  synthetic sequence
  misc_feature (1)..(1): The nucleotide at position 1 comprises Cy3
ctctctctctctctctggct

SEQ ID NO: 8 - DNA, 40 bp, Artificial sequence
  synthetic sequence
  misc_binding (1)..(1): The nucleotide at position 1 comprises AminoC6
agccagagagagagagagagctaagcagaattcccataag

SEQ ID NO: 9 - DNA, 53 bp, Artificial sequence
  synthetic sequence
  misc_binding (1)..(1): The nucleotide at position 1 comprises Cy5
cagctggatcgttacccaccctagtcattggaggtgacgagtgagtcgtatga

SEQ ID NO: 10 - DNA, 20 bp, Artificial sequence
  synthetic sequence
  misc_binding (1)..(1): The nucleotide at position 1 comprises a lipid
agtgacagctggatcgttac

SEQ ID NO: 11 - DNA, 40 bp, Artificial sequence
  synthetic sequence
actgactgactgactgactgccataagggatccctaagca

SEQ ID NO: 12 - DNA, 20 bp, Artificial sequence
  synthetic sequence
agaagaagaacgaagaagaa

SEQ ID NO: 13 - DNA, 20 bp, Artificial sequence
  synthetic sequence
agccagagagagagagagag

SEQ ID NO: 14 - DNA, 40 bp, Artificial sequence; identical to SEQ ID NO: 8
  synthetic sequence

SEQ ID NO: 15 - DNA, 50 bp, Artificial sequence
  synthetic sequence
tcatacgactcactcgtcacctccaatgactagggtgggtaacgatccag

SEQ ID NO: 16 - protein, 15 aa, Artificial sequence
  synthetic sequence
TNYLFSPNGPIARAW

SEQ ID NO: 17 - protein, 16 aa, Artificial sequence
  synthetic sequence
TNYLFSPNGPIARAWC

SEQ ID NO: 18 - protein, 16 aa, Artificial sequence
  synthetic sequence
TNYLFSPNGPIARAWC

SEQ ID NO: 19 - protein, 174 aa, Artificial sequence
  synthetic sequence
MKSSHHHHHHENLYFQSNAAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKSC

SEQ ID NO: 20 - DNA, 20 bp, Artificial sequence
  synthetic sequence
  misc_binding (1)..(1): The nucleotide at position 1 comprises AminoC6
actgactgactgactgactg

SEQ ID NO: 21 - DNA, 20 bp, Artificial sequence
  synthetic sequence
  misc_binding (1)..(1): The nucleotide at position 1 comprises an azide
  misc_binding (20)..(20): The nucleotide at position 1 comprises Cy5
cagtcagtcagtcagtcagt

SEQ ID NO: 22 - DNA, 100 bp, Artificial sequence
  synthetic sequence
  misc_binding (1)..(1): The nucleotide at position 1 comprises a lipid
gtaacgatccagctgtcactttttttttttttttttttttttttttttttttttttttttttttttttttttttttttttcagtcagtcagtcagtcagt

SEQ ID NO: 23 - DNA, 20 bp, Artificial sequence
  synthetic sequence
  misc_binding (1)..(1): The nucleotide at position 1 comprises AminoC6
agaagaagaacgaagaagaa

SEQ ID NO: 24 - DNA, 20 bp, Artificial sequence
  synthetic sequence
  misc_binding (1)..(1): The nucleotide at position 1 comprises AminoC6
ttcttcttcgttcttcttct

SEQ ID NO: 25 - DNA, 100 bp, Artificial sequence
  synthetic sequence
  misc_binding (1)..(1): The nucleotide at position 1 comprises a lipid
gtaacgatccagctgtcactttttttttttttttttttttttttttttttttttttttttttttttttttttttttttttagaagaagaacgaagaagaa

SEQ ID NO: 26 - DNA, 20 bp, Artificial sequence
  synthetic sequence
  misc_binding (1)..(1): The nucleotide at position 1 comprises AminoC6
agccagagagagagagagag

SEQ ID NO: 27 - DNA, 100 bp, Artificial sequence
  synthetic sequence
  misc_binding (1)..(1): The nucleotide at position 1 comprises a lipid
gtaacgatccagctgtcactttttttttttttttttttttttttttttttttttttttttttttttttttttttttttttctctctctctctctctggct

SEQ ID NO: 28 - DNA, 20 bp, Artificial sequence
  synthetic sequence
cttatgggaattctgcttag

SEQ ID NO: 29 - DNA, 28 bp, Artificial sequence
  synthetic sequence
agccagagagagagagagagctaagcag

SEQ ID NO: 30 - DNA, 12 bp, Artificial sequence
  synthetic sequence
aattcccataag

SEQ ID NO: 31 - DNA, 12 bp, Artificial sequence
  synthetic sequence
aattctgcttag

SEQ ID NO: 32 - DNA, 20 bp, Artificial sequence
  synthetic sequence
ctctctctctctctctggct

SEQ ID NO: 33 - DNA, 50 bp, Artificial sequence; identical to SEQ ID NO: 15
  synthetic sequence
  misc_binding (1)..(1): The nucleotide at position 1 comprises AminoC6

SEQ ID NO: 34 - DNA, 53 bp, Artificial sequence; identical to SEQ ID NO: 9
  synthetic sequence
  misc_binding (1)..(1): The nucleotide at position 1 comprises Cy5

SEQ ID NO: 35 - DNA, 40 bp, Artificial sequence; identical to SEQ ID NO: 11
  synthetic sequence
  misc_binding (1)..(1): The nucleotide at position 1 comprises AminoC6

SEQ ID NO: 36 - DNA, 20 bp, Artificial sequence
  synthetic sequence
  misc_binding (1)..(1): The nucleotide at position 1 comprises Cy5
cttatgggaattctgcttag

SEQ ID NO: 37 - DNA, 20 bp, Artificial sequence
  synthetic sequence
  misc_binding (1)..(1): The nucleotide at postion 1 comprises AminoC6
agccagagagagagagagag

(19) United States
(12) Patent Application Publication
(10) Pub. No.: US 2024/0125764 A1
(43) Pub. Date: Apr. 18, 2024

(54) COMPOSITIONS AND METHODS FOR TEMPORAL CONTROL OF CELL MODULATION

(71) Applicant: The Regents of the University of California, Oakland, CA (US)

(72) Inventors: Lydia Lee Sohn, Oakland, CA (US); David V. Schaffer, Danville, CA (US); Olivia J. Scheideler, Berkeley, CA (US); Connor Andrew Tsuchida, Berkeley, CA (US)

(21) Appl. No.: 17/768,700

(22) PCT Filed: Nov. 4, 2020
(86) PCT No.: PCT/US20/58828

§ 371 Date: Apr. 13, 2022

Related U.S. Application Data
(60) Provisional application No. 62/933,040, filed on Nov. 8, 2019.

(51) Int. Cl. G01N 33/50; C12N 9/22; C12N 15/11

(52) U.S. Cl. CPC G01N 33/5005; C12N 9/22; C12N 15/11; C12N 2310/20

(57) ABSTRACT

The present disclosure provides a composition comprising a solid support, a plurality of tethered oligonucleotides attached to the solid support, and a plurality of untethered oligonucleotides hybridized to the tethered oligonucleotides. An untethered oligonucleotide can comprise, attached via the 5′ end, a cell, or an effector molecule. Hybridization of an untethered oligonucleotide to a tethered oligonucleotide generates an enzyme cleavage site, which allows for temporally controlled removal of an effector molecule. The present disclosure provides methods of temporally modulating the activity and/or phenotype of a cell. The present disclosure provides a solid support comprising patterned tethered oligonucleotides attached thereto; and methods of making the solid support.

### CROSS-REFERENCE

This application claims the benefit of U.S. Provisional Patent Application No. 62/933,040, filed Nov. 8, 2019, which application is incorporated herein by reference in its entirety.

### STATEMENT REGARDING FEDERALLY SPONSORED RESEARCH

This invention was made with government support under Grant No. CA190843 awarded by the National Institutes of Health. The government has certain rights in the invention.

### INTRODUCTION

Biological systems are regulated by the temporal dynamics of external stimuli in which individual signals as well the coordinated actions of multiple cues are modulated across time to orchestrate tissue function and instruct cell behavior. The increasing realization of the importance of temporal parameters in signaling has motivated the development of engineering platforms for imparting temporal control, which the traditional genetic approaches for constitutive knockdown/knockouts and overexpression to perturb signaling dynamics does not provide.

There is a need in the art for systems and methods that provide for temporal control over presentation of effector proteins to cells.

### SUMMARY

The present disclosure provides a composition comprising a solid support, a plurality of tethered oligonucleotides attached to the solid support, and a plurality of untethered oligonucleotides hybridized to the tethered oligonucleotides. An untethered oligonucleotide can comprise, attached via the 5′ end, a cell, or an effector molecule. Hybridization of an untethered oligonucleotide to a tethered oligonucleotide generates an enzyme cleavage site, which allows for temporally controlled removal of an effector molecule. The present disclosure provides methods of temporally modulating the activity and/or phenotype of a cell. The present disclosure provides a solid support comprising patterned tethered oligonucleotides attached thereto; and methods of making the solid support.

### BRIEF DESCRIPTION OF THE DRAWINGS

FIG. 1A-1D show high-resolution surface DNA patterning using photolithography.

FIG. 2A-2C depict microfabricated DNA patterns direct the capture of NSCs.

FIG. 3A-3D depict scalable, multicomponent DNA patterns organize heterogeneous cell populations.

FIG. 4A-4C depict microfabricated DNA patterns direct the spatial organization of solid-phase ligands.

FIG. 5A-5C depict multicomponent DNA patterns enable tight spatial control and investigation of the presentation of competing ligand cues, FGF-2 and ephrin-B2, on single NSC behavior.

FIG. 6A-6C show cell occupancy in response to various ligand presentations of FGF-2 and ephrin-B2 and resulting end fate after 5-day differentiation.

FIG. 7A-7B show characterization and optimization of DNA Patterning Steps.

FIG. 8A-8B show optimization of DNA-Patterned Features for High Efficiency Single-Cell Capture.

FIG. 9A-9B show Re-Use of Photoresist (PR) Layer vs. New PR Layer for Multicomponent DNA Patterning.

FIG. 10 depicts robustness of Photolithographic Approach for Assembling Multiplexed DNA Patterns.

FIG. 11A-11B show tunable Multicomponent DNA Patterns within the Same and Distinct Layers.

FIG. 12A-12B show characterization of Labeling Reaction of Recombinant Fluorescent Protein, eGFP, with a Fluorescently-Tagged Oligonucleotide Label using Dibenzocyclooctyne (DBCO) Heterobifunctional Cross-linker.

FIG. 13A-13B depict Stability and Specificity of DNA-Directed Enhanced Green Fluorescent (eGFP) Protein Patterns.

FIG. 14A-14C depict optimization of Polyacrylamide (PA) Patterning using Photolithography.

FIG. 15A-15B depict microfabricated DNA and PA Patterns Support High-Throughput Clonal Analysis of Adult Neural Stem Cells (NSCs).

FIG. 16A-16B show purification of Recombinant Fibroblast Growth Factor-2 (FGF-2; SEQ ID NO:19) and 5-Ethynyl-2′-Deoxyuridine (EdU) Pulse-Chase Experiment to Validate Protein Activity in Adult Neural Stem Cells (NSCs).

FIG. 17A-17B depict characterization of Oligo Labeling Reaction of Niche Ligands, Fibroblast Growth Factor-2 (FGF-2) and EphB4-Binding Peptide, using Click Chemistry.

FIG. 18A-18B depict multicomponent DNA Patterns Enable Controlled, High-Throughput Studies of Adult Neural Stem Cell (NSC) Niche Solid-Phase Ligand Cues, Fibroblast Growth Factor-2 (FGF-2) and Ephrin-B2, at the Single-Cell Level.

FIG. 19A-19B depict Cell Body Segmentation using ilastik Software.

FIG. 20A-20C show an overview of Custom Fiji Script for Counting Cells.

FIG. 21A-21B depict tracking changes in Average Cell Occupancy within FGF-2 over Time for Each Individual Microisland.

FIG. 22A-22C show polydimethylsiloxane (PDMS) Stamping Protocol to Enable Cell Patterning.

FIG. 23A-23B depict DNA-Directed Cell Patterning using Photolithographically-Defined Surface DNA Patterns.

FIG. 24 shows an overview of Surface-Patterned DNA Sequences and their Complementary Fluorescent, Cell-Labeling, and Ligand-Labeling Oligonucleotides.

FIG. 25 shows an overview of Complementary Pairs of DNA Strands used for Characterization, Cell Patterning, Ligand Patterning, and Biological Experiments.

FIG. 26 shows an in-Depth Report of Experimental Sample Number “n”.

FIG. 27A-27B show a DNase-based strategy to achieve rapid, one-pot cleavage.

FIG. 28A-28B show DNase-based cleavage of patterned fibroblast growth factor-2 (FGF-2) induced changes in single NSC proliferation.

FIG. 29A-29C show Encoding DNA cleavage specificity with restriction sites.

FIG. 30A-30C depict RNA-guided, site-specific cleavage with Streptococcus pyogenes Cas9 (spCas9) ribonucleoprotein (RNP).

FIG. 31 depicts DNA-Patterned, High-Throughput Single Neural Stem Cell (NSC) Array.

FIG. 32 depicts Patterned Single Neural Stem Cell (NSC) Retention Following Laminin Incubation and DNase Treatment.

FIG. 33 shows the effects of DNase treatment on adult neural stem cell (NSC) differentiation.

FIG. 34 depicts Live/Dead Assessment of Adult Neural Stem Cells (NSCs) following 1× CutSmart Treatment in N2 Media for Various Incubation Times.

FIG. 35A-35B show the effects of 1× CutSmart buffer on NSC behavior.

FIG. 36A-36B depict Cleavage Kinetics of Plasmid in Different Buffer Conditions using Gel Format.

FIG. 37A-37B depict specificity of Restriction Enzyme-Based Cleavage.

FIG. 38 depicts in-Depth Visualization of Rehybridization Strategy Post Restriction Enzyme-Based Cleavage.

FIG. 39 depicts SpCas9 RNP Cleavage Kinetics Screen in Gel Format.

FIG. 40 depicts an overview of DNA Sequences.

FIG. 41A-41D depict RNA-guided, site-specific cleavage with Streptococcus pyogenes Cas9 (spCas9) ribonucleoprotein (RNP).

FIG. 42A-42B depict engineering complex temporal signaling logic.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an effector molecule” includes a plurality of such effector molecules and reference to “the target cell” includes reference to one or more target cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a composition comprising a solid support, a plurality of tethered oligonucleotides attached to the solid support, and a plurality of untethered oligonucleotides hybridized to the tethered oligonucleotides. An untethered oligonucleotide can comprise, attached via the 5′ end, a cell, or an effector molecule. Hybridization of an untethered oligonucleotide to a tethered oligonucleotide generates an enzyme cleavage site, which allows for temporally controlled removal of an effector molecule. The present disclosure provides methods of temporally modulating an activity and/or phenotype of a cell. The present disclosure provides a solid support comprising patterned tethered oligonucleotides attached thereto; and methods of making the solid support.

### Compositions

The present disclosure provides a composition comprising a solid support, a plurality of tethered oligonucleotides attached to the solid support, and a plurality of untethered oligonucleotides hybridized to the tethered oligonucleotides. An untethered oligonucleotide can comprise, attached via the 5′ end, a cell, or an effector molecule. Hybridization of an untethered oligonucleotide to a tethered oligonucleotide generates an enzyme cleavage site, which allows for temporally controlled removal of an effector molecule.

A composition of the present disclosure can comprise: a) a solid support; b) a plurality of tethered oligonucleotides, where the tethered oligonucleotides are attached to the solid support via the 5′ termini of the oligonucleotides, and where each of the plurality of tethered oligonucleotides comprises a nucleotide sequence that, when hybridized to a complementary nucleotide sequence present in an untethered oligonucleotide, generates an enzyme cleavage site; and c) a plurality of untethered oligonucleotides that are hybridized to the plurality of tethered oligonucleotides, where the untethered oligonucleotides each comprises: i) the nucleotide sequence that generates an enzyme cleavage site (e.g., where the enzyme cleavage site is a restriction enzyme cleavage site or a site that is cleavable by a CRISPR/Cas effector polypeptide); ii) either a cell, or an effector molecule that affects an activity and/or a phenotype of a cell, where the cell or the effector molecule is attached to the untethered oligonucleotide at the 5′ end of the untethered oligonucleotides. In some cases, the untethered oligonucleotides each include, attached thereto, a fluorophore.

A composition of the present disclosure can comprise: a) a solid support; b) a plurality of tethered oligonucleotides, where the tethered oligonucleotides are attached to the solid support via the 5′ termini of the oligonucleotides in a patterned array, wherein each of the plurality of tethered oligonucleotides comprises a nucleotide sequence that, when hybridized to a complementary nucleotide sequence present in an untethered oligonucleotide, generates an enzyme cleavage site; and c) a plurality of untethered oligonucleotides that are hybridized to the plurality of tethered oligonucleotides in the patterned array, wherein the untethered oligonucleotides each comprise: i) the nucleotide sequence that generates an enzyme cleavage site, wherein the enzyme cleavage site is a restriction enzyme cleavage site or a site that is cleavable by a CRISPR/Cas effector polypeptide; ii) a cell, or an effector molecule that affects an activity and/or a phenotype of a cell, wherein the cell or the effector molecule is attached to the untethered oligonucleotide at the 5′ end of the untethered oligonucleotides; and iii) a fluorophore. The composition can comprise multiple subsets (also referred to herein as “micro-islands”) in a patterned array, where each of the micro-islands comprises: i) at least one untethered oligonucleotide to which a cell is attached; and ii) two or more (e.g., 2, 3, 4, 5, 6, 7, 8, 9, 10, or more than 10) untethered oligonucleotides, where a different effector molecule (e.g., 2, 3, 4, 5, 6, 7, 8, 9, 10, or more than 10 different effector molecules) is attached to each of the two or more untethered oligonucleotides, and where the two or more untethered oligonucleotides are arranged in a patterned array around the untethered oligonucleotide to which the cell (the target cell) is attached. In this manner, the two or more effector molecules are spatially arranged relative to the target cell. The spatial arrangement of the two or more effector molecules relative to the target cell can recapitulate the spatial arrangement of such effector molecules, relative to the target cell, as it might occur physiologically, e.g., in a tissue or organ.

The tethered and the untethered oligonucleotides have a length of from about 15 nucleotides (nt) to about 50 nt; e.g., the tethered and the untethered oligonucleotides have a length of from about 15 nt to about 20 nt (e.g., 15 nt, 16 nt, 17 nt, 18 nt, 19 nt, or 20 nt), from about 20 nt to about 25 nt (e.g., 20 nt, 21 nt, 22 nt, 23 nt, 24 nt, or 25 nt), from about 25 nt to about 30 nt (e.g., 25 nt, 26 nt, 27 nt, 28 nt, 29 nt, or 30 nt), from about 30 nt to about 35 nt, from about 35 nt to about 40 nt, from about 40 nt to about 45 nt, or from about 45 nt to about 50 nt.

As noted above, each of the plurality of tethered oligonucleotides comprises a nucleotide sequence that, when hybridized to a complementary nucleotide sequence present in an untethered oligonucleotide, generates an enzyme cleavage site. Such a nucleotide sequence is referred to herein as a “hybridization nucleotide sequence.” The length of the hybridization nucleotide sequence can be from about 6 nucleotides (nt) to about 30 nt; e.g., the length of the hybridization nucleotide sequence can be from about 6 nt to about 10 nt (e.g., 6 nt, 7 nt, 8 nt, 9 nt, or 10 nt), from about 10 nt to about 15 nt (e.g., 10 nt, 11 nt, 12 nt, 13 nt, 14 nt, or 15 nt), from about 15 nt to about 20 nt (e.g., 15 nt, 16 nt, 17 nt, 18 nt, 19 nt, or 20 nt), from about 20 nt to about 25 nt, or from about 25 nt to about 30 nt.

In some cases, the tethered oligonucleotides comprise a hybridization nucleotide sequence that, when hybridized to a complementary nucleotide sequence present in an untethered oligonucleotide, generates a restriction enzyme recognition site. In some cases, the tethered oligonucleotides comprise a hybridization nucleotide sequence that, when hybridized to a complementary nucleotide sequence present in an untethered oligonucleotide, generates an enzyme cleavage site that can be cleaved by a CRISPR/Cas effector polypeptide when the CRISPR/Cas effector polypeptide is complexed with a guide RNA that comprises a nucleotide sequence that is complementary to the hybridization nucleotide sequence present in the tethered oligonucleotide or the untethered oligonucleotide. Non-limiting examples of untethered oligonucleotides comprising a nucleotide sequence include, but are not limited to: A′-BamHI-Cy3: [Cy3] CAGTCAGTCAGTCAGTCAGT (SEQ ID NO:1); A′-Cy5: [Cy5] CAGTCAGTCAGTCAGTCAGT(SEQ ID NO:2); F′-AlexaFluor 488: [AlexaFluor488] TTCTTCTTCGTTCTTCTTCT(SEQ ID NO:3); F′-Lipid: [Lipid GTAACGATCCAGCTGTCACTTTTTTTTTTTTTTTTTTTTTTTTTTTTTTTTTTTTTTTTTTTTTTT TTTTTTTTTTTTTTTTCTTCTTCGTTCTTCTTCT(SEQ ID NO:4); Azide-G′-Cy3: [Azide] CTCTCTCTCTCTCTCTGGCT [Cy3] (SEQ ID NO:5); G′-Cy5: [Cy5] CTCTCTCTCTCTCTCTGGCT (SEQ ID NO:6); G′-Cy3: [Cy3] CTCTCTCTCTCTCTCTGGCT (SEQ ID NO:7); G-EcoRI-NH₂: [AminoC6] AGCCAGAGAGAGAGAGAGAGCTAAGC AGAATTCCCATAAG (SEQ ID NO:8); G′-EcoRI-Cy5: [Cy5] CAGCTGGATCGTTACCCACCCTAGTCATTGGAGGTGACGAGTGAGTCGTATGA (SEQ ID NO:9); and CoAnchor Lipid: [Lipid] AGTGACAGCTGGATCGTTAC (SEQ ID NO:10).

In some cases, the one or more tethered oligonucleotides is a 5′ amine-modified oligonucleotide. In some cases, the one or more tethered oligonucleotides comprises a nucleotide sequence that, when hybridized to a complementary nucleotide sequence present in an untethered oligonucleotide, generates an enzyme cleavage site. Non-limiting examples of nucleotide sequences of the tethered oligonucleotides, include, but are not limited to: A-BamHI-NH₂: ACTGACTGACTGACTGACTGCCATAAGGGATCCCTAAGCA (SEQ ID NO:11); F—NH₂: AGAAGAAGAACGAAGAAGAA (SEQ ID NO:12); G-NH₂: AGCCAGAGAGAGAGAGAGAG (SEQ ID NO:13); G-EcoRI-NH₂ AGCCAGAGAGAGAGAGAGAGCTAAGC AGAATTCCCATAAG (SEQ ID NO:14); B-Cas9-PAM-D′-NH₂, and TCATACGACTCACTCGTCACCTCCAATGACTAGGGTGGGTAACGATCCAG (SEQ ID NO:15).

In some cases, the one or more tethered oligonucleotides are covalently attached to an aldehyde-reactive substrate within the one or more patterns, thereby forming a tethered DNA patterned substrate. The one or more tethered DNA patterns comprises a diameter ranging from 50 nm to 50 mm. In some cases, the one or more tethered DNA patterns comprises a diameter ranging from 50 nm to 100 nm. In some cases, the one or more tethered DNA patterns comprises a diameter ranging from 50 nm to 100 μm. In some cases, the one or more tethered DNA patterns comprises a diameter ranging from 2 to 5 μm, 5 to 10 μm, 10 to 15 μm, 15 to 20 μm, 20 to 25 μm, 25 to 30 μm, 30 to 35 μm, 35 to 40 μm, 40 to 45 μm, or 45 to 50 μm. In some cases, the one or more tethered DNA patterns comprises a diameter ranging from 50 to 100 μm, 100 to 150 μm, 150 to 200 μm, 200 to 250 μm, 250 to 300 μm, 300 to 350 μm, 350 to 400 μm, 400 to 450 μm, or 450 to 500 μm. In some cases, the one or more tethered DNA patterns comprises a diameter ranging from 1 mm to 5 mm, 5 mm to 10 mm, 10 mm to 15 mm, 15 mm to 20 mm, 20 mm to 25 mm, or 25 mm to 30 mm.

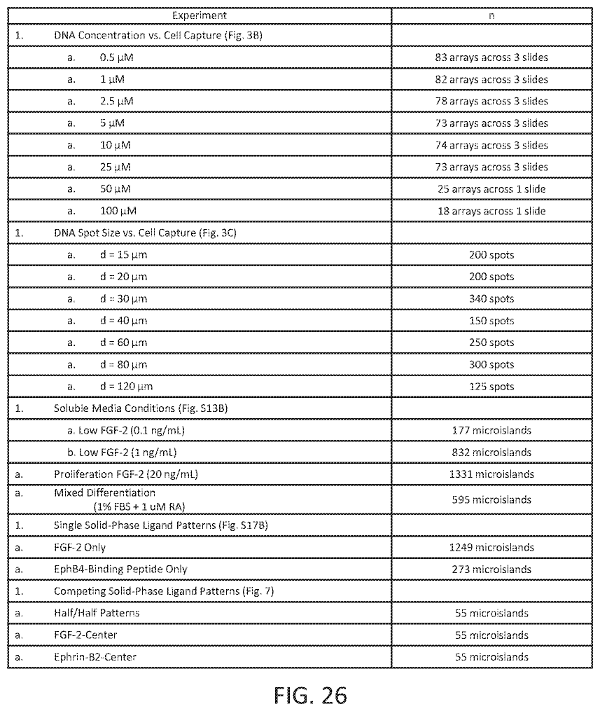

In some cases, the one or more tethered DNA patterns is a cylindrical shape, a circular shape, a square shape, a spherical shape, a cylindrical shape, a rectangular shape, or a combination thereof. In some cases, the one or more tethered DNA patterns comprises one or more micro-islands. In some cases, the one or more tethered DNA patterns comprises one or more tethered DNA patterns selected from the tethered DNA patterns as shown in FIG. 26. The one or more tethered DNA patterns is not limited to the shapes and/or sizes as described herein and can be any shape and/or size as required per conditions specific to its intended use.

In some cases, the one or more pattern comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, 500 or more, 1000 or more, 1500 or more, or 2000 or more tethered DNA patterns.

In some cases, the DNA pattern comprises two or more array of tethered DNA patterns, three or more array of tethered DNA patterns, four or more array of tethered DNA patterns, five or more array of tethered DNA patterns, six or more array of tethered DNA patterns, seven or more array of tethered DNA patterns, eight or more array of tethered DNA patterns, nine or more array of tethered DNA patterns, or ten or more array of tethered DNA patterns separated from one another. In some cases, an array of tethered DNA patterns comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, 500 or more, 1000 or more, 1500 or more, or 2000 or more tethered DNA patterns. In some cases, the grid separates a first array of tethered DNA patterns from a second array of tethered DNA patterns. In some cases, the aldehyde-reactive substrate comprises a 2×2 array of tethered DNA patterns, a 3×3 array of tethered DNA patterns, a 4×4 array of tethered DNA patterns, a 5×5 array of tethered DNA patterns, a 6×6 array of tethered DNA patterns, a 7×7 array of DNA patterns, an 8×8 array of tethered DNA patterns, a 9×9 array of tethered DNA patterns, or a 10×10 array of tethered DNA patterns. In some cases, the aldehyde-reactive substrate comprises a 15×15 array of tethered DNA patterns, a 20×20 array of tethered DNA patterns, a 25×25 array of tethered DNA patterns, a 100×100 array of tethered DNA patterns, a 150×150 array of tethered DNA patterns, or more than 150×150 array of tethered DNA patterns. In some cases, the grid creates one or more micro-islands separated from one another, each comprising an array of tethered DNA patterns. In some cases, the one or more microislands is a cylindrical shape, a circular shape, a square shape, a spherical shape, a cylindrical shape, a rectangular shape, or a combination thereof. In some cases, the one or more microislands comprises a length ranging from 1 μm to 500 μm and a width ranging from 1 μm to 500 μm. In some cases, the one or more microislands comprises a length of 141 μm and a width of 141 μm. In some cases, the one or more microislands comprises a length ranging from 1 mm to 50 mm and a width ranging from 1 mm to 50 mm. In some cases, the one or more microislands comprises one or more microislands selected from the microislands as shown in FIG. 26. The one or more microislands comprising one or more tethered DNA patterns is not limited to the shapes and/or sizes as described herein and can be any shape and/or size as required per conditions specific to its intended use.

In some cases, a composition of the present disclosure comprises: at least a first, a second, and a third plurality of hybridized, untethered oligonucleotides that are bound to the tethered oligonucleotides in the patterned array, wherein: a) the first plurality of hybridized, untethered oligonucleotides comprises, bound to the 5′ end of the oligonucleotides, a first effector molecule, wherein the first plurality of hybridized, untethered oligonucleotides generates a first enzyme cleavage site; and b) the second plurality of hybridized, untethered oligonucleotides comprises, bound to the 5′ end of the oligonucleotides, a second effector molecule, wherein the second plurality of hybridized, untethered oligonucleotides generates a second enzyme cleavage site; and c) the third plurality of hybridized, untethered oligonucleotides comprises, bound to the 5′ end of the oligonucleotides, a target cell. In some cases, the composition further comprises a fourth plurality of hybridized, untethered oligonucleotides, wherein the fourth plurality of hybridized, untethered oligonucleotides bound to the 5′ end of the oligonucleotides, a third effector molecule, wherein the fourth plurality of hybridized, untethered oligonucleotides generates a third enzyme cleavage site. In some cases, the composition further comprises a fifth plurality of hybridized, untethered oligonucleotides, wherein the fifth plurality of hybridized, untethered oligonucleotides bound to the 5′ end of the oligonucleotides, a fourth effector molecule, wherein the fifth plurality of hybridized, untethered oligonucleotides generates a fourth enzyme cleavage site. In some cases, the composition further comprises a sixth plurality of hybridized, untethered oligonucleotides, wherein the sixth plurality of hybridized, untethered oligonucleotides bound to the 5′ end of the oligonucleotides, a fifth effector molecule, wherein the sixth plurality of hybridized, untethered oligonucleotides generates a fifth enzyme cleavage site. In some cases, the composition further comprises a seventh plurality of hybridized, untethered oligonucleotides, wherein the seventh plurality of hybridized, untethered oligonucleotides bound to the 5′ end of the oligonucleotides, a sixth effector molecule, wherein the seventh plurality of hybridized, untethered oligonucleotides generates a sixth enzyme cleavage site. In some cases, the composition further comprises an eighth plurality of hybridized, untethered oligonucleotides, wherein the eighth plurality of hybridized, untethered oligonucleotides bound to the 5′ end of the oligonucleotides, a seventh effector molecule, wherein the eighth plurality of hybridized, untethered oligonucleotides generates a seventh enzyme cleavage site. In some cases, the composition further comprises a ninth plurality of hybridized, untethered oligonucleotides, wherein the ninth plurality of hybridized, untethered oligonucleotides bound to the 5′ end of the oligonucleotides, an eighth effector molecule, wherein the ninth plurality of hybridized, untethered oligonucleotides generates an eighth enzyme cleavage site. In some cases, the composition further comprises a tenth plurality of hybridized, untethered oligonucleotides, wherein the tenth plurality of hybridized, untethered oligonucleotides bound to the 5′ end of the oligonucleotides, a ninth effector molecule, wherein the tenth plurality of hybridized, untethered oligonucleotides generates a ninth enzyme cleavage site.

### Restriction Enzyme Cleavage Sites

In some cases, the tethered oligonucleotides comprise a hybridization nucleotide sequence that, when hybridized to a complementary nucleotide sequence present in an untethered oligonucleotide, generates a restriction enzyme recognition site. Numerous restriction enzymes, and their corresponding recognition sites, are known in the art. See, e.g., Roberts et al. (2014) Nucl. Acids Res. 43(D1):D298. Examples of restriction enzyme cleavage sites include nucleotide sequences recognized and cleaved by, e.g., EcoRI, BamHI, HindIII, KpnI, NotI, PstI, SmaI, XhoI, NaeI, NarI, BspMI, HpaII, Sa II, EcoRII, AtuBI, Cfr9I, SauBMKI, FokI, Alw26I, BbvI, BsrI, Earl, HphI, MboII, SfaNI, and the like.

### CRISRP/Cas Effector Polypeptides

In some cases, the tethered oligonucleotides comprise a hybridization nucleotide sequence that, when hybridized to a complementary nucleotide sequence present in an untethered oligonucleotide, generates a site that is cleaved by a CRISPR/Cas effector polypeptide when the CRISPR/Cas effector polypeptide is complexed with a guide RNA that comprises a nucleotide sequence that hybridizes to the hybridization nucleotide sequence in the tethered oligonucleotide or to the complementary nucleotide sequence in the untethered oligonucleotide.

Examples of suitable CRISPR/Cas effector polypeptides include class 2 CRISPR/Cas effector polypeptides, such as a type II CRISPR/Cas effector polypeptides, type V CRISPR/Cas effector polypeptides, and type VI CRISPR/Cas effector polypeptides.

In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR/Cas protein” is used herein to encompass the CRISPR/Cas effector polypeptide (e.g., the target nucleic acid cleaving protein) from class 2 CRISPR systems. Thus, the term “class 2 CRISPR/Cas effector polypeptide” as used herein encompasses type II CRISPR/Cas effector polypeptides (e.g., Cas9); type V-A CRISPR/Cas effector polypeptides (e.g., Cpf1 (also referred to a “Cas12a”)); type V-B CRISPR/Cas effector polypeptides (e.g., C2c1 (also referred to as “Cas12b”)); type V-C CRISPR/Cas effector polypeptides (e.g., C2c3 (also referred to as “Cas12c”)); type V-U1 CRISPR/Cas effector polypeptides (e.g., C2c4); type V-U2 CRISPR/Cas effector polypeptides (e.g., C2c8); type V-U5 CRISPR/Cas effector polypeptides (e.g., C2c5); type V-U4 CRISPR/Cas proteins (e.g., C2c9); type V-U3 CRISPR/Cas effector polypeptides (e.g., C2c10); type VI-A CRISPR/Cas effector polypeptides (e.g., C2c2 (also known as “Cas13a”)); type VI-B CRISPR/Cas effector polypeptides (e.g., Cas13b (also known as C2c4)); and type VI-C CRISPR/Cas effector polypeptides (e.g., Cas13c (also known as C2c7)). To date, class 2 CRISPR/Cas effector polypeptides encompass type II, type V, and type VI CRISPR/Cas effector polypeptides, but the term is also meant to encompass any class 2 CRISPR/Cas effector polypeptide suitable for binding to a corresponding guide RNA and forming an RNP complex.

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual- (dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, which are repaired either by non-homologous end joining (NHEJ) or homology-directed recombination (HDR).

Examples of various Cas9 proteins (and Cas9 domain structure) and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

### Cells Bound to Untethered Oligonucleotides

As noted above, the untethered oligonucleotides comprise: i) a cell; or ii) an effector molecule that affects an activity and/or a phenotype of a cell, where the cell or the effector molecule is attached to the untethered oligonucleotide at the 5′ end of the untethered oligonucleotides. As noted above, in some cases, a composition comprises: i) an untethered oligonucleotide comprising, attached thereto, a target cell; and ii) two or more (e.g., 2, 3, 4, 5, 6, 7, 8, 9, or 10, or more than 10) different untethered oligonucleotides (generating different enzyme cleavage sites) each having attached thereto a different effector molecule.

Cells that can be bound to untethered oligonucleotides include eukaryotic cells. Cells that can be bound to untethered oligonucleotides include mammalian cells, insect cells, arachnid cells, amphibian cells, reptile cells, avian cells, fish cells, plant cells, algal cells, helminth cells, protozoan cells, and the like. Mammalian cells that can be bound to untethered oligonucleotides include human cells, non-human primate cells, ungulate cells (e.g., bovine cells; ovine cells; caprine cells; equine cells; etc.), rodent cells (e.g., murine cells such as mouse cells and rat cells), lagomorph cells, feline cells, canine cells, and the like.

Where the cell is an animal cell (e.g., a mammalian cell), suitable cells include cells of particular tissues (e.g., lung, liver, heart, kidney, brain, pancreas, muscle (e.g., cardiac muscle; skeletal muscle), spleen, skin, fetal tissue, etc.), or a particular cell type (e.g., neuronal cells, epithelial cells, endothelial cells, immune cells, leukocytes, fibroblasts, astrocytes, macrophages, glial cells, dendritic cells, smooth muscle cells, skeletal muscle cells, islet cells, T lymphocytes, B lymphocytes, etc.). In some cases, the cell is a primary cell (e.g., a cell obtained from an individual). In some cases, the cell is a cell line (e.g., an immortalized cell line).

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some cases, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In some cases, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In some cases, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some cases, the cell is a diseased cell. Diseased cells include, e.g., cancerous cells; virus-infected cells; bacterium-infected cells; protozoan-infected cells; cells comprising a pathology-associated mutation (e.g., sickle cell anemia; Huntington's disease; and the like); cells from an individual with a non-Mendelian disorder (e.g., cells from an individual with Crohn's disease; cells from an individual with idiopathic pulmonary fibrosis; etc.).

Suitable cancer cells include, but are not limited to, cancerous cells of any of the following cancers: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epithelioid cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some cases, the effect of an effector molecule on an activity and/or phenotype of a cancer cell is compared to the effect of the effector molecule on the activity and/or phenotype of a non-cancerous cell. The non-cancerous (“normal”) cell can be a cell of the same cell type as the cancerous cell.

### Effector Molecules Bound to Untethered Oligonucleotides

As noted above, the untethered oligonucleotides comprise: i) a cell; or ii) an effector molecule that affects an activity and/or a phenotype of a cell, where the cell or the effector molecule is attached to the untethered oligonucleotide at the 5′ end of the untethered oligonucleotides.

Effector molecules include polypeptides, lipids, and oligosaccharides. Effector molecules include molecules comprising both a polypeptide and a lipid. Effector molecules include molecules comprising both a polypeptide and an oligosaccharide. Effector molecules include molecules comprising both a lipid and an oligosaccharide.

Effector molecules comprising a lipid include, e.g., effector molecules comprising a fatty acyl; a sterol lipid (e.g., a sterol (e.g., cholesterol, ergosterol, C24-propyl sterols, or stanol), a steroid, a secosteroid, or bile acid); a lycerolipid; a glycerophospholipid; a sphingolipid; a sterol lipid; a prenol lipid; a saccharolipid; a polyketide; lysophosphatidic acid; sphingosine 1-phosphate; phosphatidyl choline; sphingomyelin; lysophospholipids; cannabinoids; sn-2-arachidonoylglycerol; prostanoids (e.g., prostaglandins, thromboxanes, prostacyclins); leukotrienes; a fatty acid (e.g., a saturated fatty acid (e.g., propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, or hexatriacontanoic acid), a monounsaturated fatty acid (e.g., palmitoleic acid, vaccenic acid, oleic acid, eicosenoic acid, erucic acid, gadoleic acid, myristoleic acid, or nervonic acid), or a polyunsaturated fatty acid (e.g., hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, tetracosahexaenoic acid, linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid, mead acid, rumenic acid, alpha-calendic acid, beta-calendic acid, jacaric acid, alpha-eleostearic acid, beta-eleostearic acid, catalpic acid, punicic acid, rumelenic acid, alpha-parinaric acid, beta-parinaric acid, bosseopentaenoic acid, pinolenic acid, or podocarpic acid)); and the like.

Effector molecules comprising an oligosaccharide include, e.g., effector molecules comprising mannose; N-acetylglucosamine; galactose; galactosamine; glucosamine; fucose; a sialyl Lewis X glycan such as a sialyl-Lewis^{x} (sLe^{x}) tetrasaccharide; a ganglioside; and the like. See. e.g., U.S. 2018/0055928.

### Effector Polypeptides

Suitable effector polypeptides include, but are not limited to, growth factors, hormones (e.g., peptide hormones), adhesion proteins, tumor-associated antigens, integrins, chemokines, juxtacrines, antibodies, extracellular matrix polypeptides, co-stimulatory polypeptides, T-cell receptors, leukotrienes, morphogens (e.g., Wnt polypeptides), delta family proteins, Notch family proteins, Eph/Ephrins; and the like.

Non-limiting examples of suitable effector polypeptides include adrenocorticotropic hormone (ACTH), amylin, angiopoietin-1, angiopoietin-2, angiotensin, atrial natriuretic peptide (ANP), bone-derived growth factor (BDGF), bone morphogenic protein (BMP) (e.g., BMP-2, BMP-4, BMP-7), brain-derived neurotrophic factor (BDNF), calcitonin, cartilage-derived growth factor (CDGF), cholecystokinin, ephrin B2 (EphB2), epidermal growth factor (EGF), erythropoietin, fibroblast growth factor (FGF), FGF-2, FGF-19, FGF-3, FGF-8, follicle stimulating hormone (FSH), gastrin, glial fibrillary acidic protein (GFAP), glucagon, granulocyte colony stimulating factor (G-CSF), granulocyte-macrophage colony stimulating factor (GM-CSF), ghrelin, hematopoietic growth factor, hepatocyte growth factor (HGF), insulin, insulin-like growth factor (IGF1), an interleukin (IL) (e.g., IL-1, IL-1, IL-4, IL-5, IL-6, IL-8, IL-9, IL-10, IL-12, IL-13, IL-15, IL-18, IL-23), an interferon (IFN) (e.g., IFN-α, IFN-β, IFN-γ), keratinocyte growth factor, leptin, leuteinizing hormone, macrophage inflammatory peptide MIP-la, macrophage inflammatory peptide MIP-1b, macrophage colony stimulating factor (M-CSF), macrophage chemoattractant and activating factor (MCAF), melanocyte-stimulating hormone (MSH), nerve growth factor (NGF), neural epidermal growth factor-like-1 (NELL1), neurotrophin-3 (NT-3), neurotrophin-4 (NT-4), neuronal growth factor, neutrophil activating protein (NAP), a Notch polypeptide (e.g., Notch-1, Notch-2, Notch-3, Notch-4), oxytocin, parathyroid hormone, parathyroid hormone-related peptide (PTHrP), placental growth factor, platelet derived growth factor (PDGF), prolactin, RANTES, relaxin, renin, resistin, skeletal growth factor (SGF), somatostatin, stromal derived growth factor 1 (SDF-1), thyroid stimulating hormone (TSH), thyrotropin-releasing hormone, transforming growth factor (TGF)-β1, TGF-β2, TGF-β3, TGF-β4, TGF-β5, tumor necrosis factor (TNF), thrombopoietin, vascular endothelial growth factor (VEGF), vasopressin, vasoactive intestinal peptide, a Wnt polypeptide (e.g., Wnt1, Wnt2, Wnt2b (also called Wnt13), Wnt3, Wnt3a, Wnt4, Wnt5a, Wnt5b, Wnt6, Wnt7a, Wnt7b, Wnt7c, Wnt8, Wnt8a, Wnt8b, Wnt5c, Wnt10a, Wnt10b, Wnt11, Wnt14, Wnt15, or Wnt16), and the like.

Non-limiting examples of suitable effector polypeptides include laminin, integrin, collagen, elastin, tenascin, fibronectin, fibrinogen, poly(L-lysine), vitronectin, fibrillin, thrombospondin, and the like.

Suitable effector polypeptides include antibodies. The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein.

In some cases, the effector polypeptide is a nanobody. The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

In some cases, the effector polypeptide is a single-chain Fv (scFv). “Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

### Attaching an Effector Molecule to an Oligonucleotide

An effector molecule (polypeptide; lipid; oligosaccharide) can be attached to the 5′ end of an untethered oligonucleotide using any of a variety of methods. Methods of attaching molecules (e.g., polypeptide; lipid; oligosaccharide) to nucleic acids are known in the art; any such method can be used to attach an effector molecule (polypeptide; lipid; oligosaccharide) to an untethered oligonucleotide. The oligonucleotide can comprise, or can be modified to comprise, a first reactive coupling group; and the effector molecule (polypeptide; lipid; oligosaccharide) can comprise, or can be modified to comprise, a second reactive coupling group. The first reactive coupling group is capable of reacting with the second reactive coupling group to form a covalent bond.

Suitable attachment chemistries include: 1) reaction of acrylamides, alkyl halides, alkyl sulfonates, aziridines, haloacetamides, or maleimides with thiols to form thioether bonds; 2) reaction of acyl halides, acyl nitriles, anhydrides, or carboxylic acids with alcohols or phenols to form an ester bond; 3) reaction of an aldehyde with an amine or aniline to form an imine bond; 4) reaction of an aldehyde or ketone with a hydrazine to form a hydrazone bond; 5) reaction of an aldehyde or ketone with a hydroxylamine to form an oxime bond; 6) reaction of an alkyl halide with an amine or aniline to form an alkyl amine bond; 7) reaction of alkyl halides, alkyl sulfonates, diazoalkanes, or epoxides with carboxylic acids to form an ester bond; 8) reaction of an alkyl halide or alkyl sulfonate with an alcohol or phenol to form an ether bond; 9) reaction of an anhydride with an amine or aniline to form a carboxamide or imide bond; 10) reaction of an aryl halide with a thiol to form a thiophenol bond; 11) reaction of an aryl halide with an amine to form an aryl amine bond; 12) reaction of a boronate with a glycol to form a boronate ester bond; 13) reaction of a carboxylic acid with a hydrazine to form a hydrazide bond; 14) of a carbodiimide with a carboxylic acid to form an N-acylurea or anhydride bond; 15) reaction of an epoxide with a thiol to form a thioether bond; 16) reaction of a haloplatinate with an amino or heterocyclic group to form a platinum complex; 16) reaction of a halotriazine with an amine or aniline to form an aminotriazine bond; 17) reaction of a halotriazines with an alcohol or phenol to form a triazinyl ether bond; 18) reaction of an imido ester with an amine or aniline to form an amidine bond; 19) reaction of an isocyanate with an amine or aniline to form a urea; 20) reaction of an isocyanate with an alcohol or phenol to form a urethane bond; 21) reaction of an isothiocyanate with an amine or aniline to form a thiourea bond; 22) reaction of a phosphoramidate with an alcohol to form a phosphite ester bond; 23) reaction of a silyl halide with an alcohol to form a silyl ether bond; 24) reaction of a sulfonate ester with an amine or aniline to form an alkyl amine bond; 25) reaction of a sulfonyl halide with an amine or aniline to form a sulfonamide bond; 26) reaction of a thioester with the thiol group of a cysteine followed by rearrangement to form an amide bond; 27) reaction of an azide with an alkyne to form a 1,2,3-triazole; and 28) of an aldehyde with an N-terminal cysteine to form a 5-membered thiazolidine ring.

### Fluorophores and Chromophores

A fluorophore or a chromophore can be attached to the 3′ end of an untethered oligonucleotide. Any of a variety of chromophores and fluorophores can be attached to an oligonucleotide. Suitable chromophores and fluorophores include, but are not limited to, 3,3′-Diaminobenzidine (DAB), 3-Amino-9-Ethylcarbazole (AEC), 4-Chloro-1-Naphtol (CN), P-Phenylenediamine Dihydrochloride/pyrocatechol (Hanker-Yates reagent), Fast Red TR, New Fuchsin, Fast Blue BB, and the like. Suitable fluorescent compounds include, but are not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AutoFluorescent Protein; Alexa Fluor350™; Alexa Fluor430™; Alexa Fluor488™; Alexa Fluor 532™; Alexa Fluor546™; Alexa Fluor568™; Alexa Fluor594™; Alexa Fluor 633™; Alexa Fluor 647™; Alexa Fluor660™; Alexa Fluor680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H; Blue Fluorescent Protein); BFP/GFP FRET; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Brilliant Violet 421; Brilliant Violet 510; Brilliant Violet 605; Brilliant Violet 650; Brilliant Violet 711; Brilliant Violet 786; BTC; BTC-SN; Calcein; Calcein Blue; Calcium Crimson™; Calcium Green; Calcium Green-1; Calcium Green-2; Calcium Green-SN; Calcium Green-C18; Calcium Orange; Calcofluor White; Carboxy-X-hodamine (5-ROX); Cascade Blue™; Cascade Yellow; Catecholamine; CCF2 (GeneBlazer); CFDA; CFP (Cyan Fluorescent Protein); CF405S; CF488A; CF 488; CF 543; CF 647; CF 750; CF 760; CF 780; FP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS; DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; Dopamine; DsRed; DTAF; DY-630-NHS; DY-635-NHS; EBFP (Enhanced Blue Fluorescent Protein); ECFP (Enhanced Cyan Fluorescent Protein); EGFP (Enhanced Green Fluorescent Protein); ELF 97; Eosin; ER-Tracker™ Green; ER-Tracker™ Red; ER-Tracker™ Blue-White DPX; Erythrosin; Erythrosin ITC; Ethidium Bromide; Ethidium homodimer-1 (EthD-1); Euchrysin; EukoLight; Europium (III) chloride; EYFP (Enhanced Yellow Fluorescent Protein); Fast Blue; FDA; FIF (Formaldehyde Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green; Mitotracker Orange; Mitotracker Red; Mitramycin; Monobromobimane; Monobromobimane (mBBr-GSH); Monochlorobimane; MPS (Methyl Green Pyronine Stilbene); mStrawberry; NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B; Phycoerythrin R; PKH26 (Sigma); PKH67; PMIA; Pontochrome Blue Black; POPO-1; POPO-3; PO-PRO-1; PO-PRO-3; Primuline; Procion Yellow; Propidium Iodid (PI); Pyrene; Pyronine; Pyronine B; Pyrozal Brilliant Flavin 7GF; QD400; QD425; QD450; QD500; QD520; QD525; QD530; QD535; QD540; QD545; QD560; QD565; QD570; QD580; QD585; QD590; QD600; QD605; QD610; QD620; QD625; QD630; QD650; QD655; QD705; QD800; QD1000; QSY 7; Quinacrine Mustard; Red 613 (PE-TexasRed); Resorufin; RFP; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin; rsGFP (red shifted GFP (S65T)); S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgGFP™ (super glow GFP; SITS (Primuline); SITS (Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; SYTOX Red; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TetramethylRodaminelsoThioCyanate; True Blue; TruRed; Tubulin Tracker™ Green; Ultralite; Uranine B; Uvitex SFC; wt GFP (wild type GFP); WW 781; X-Rhodamine; XRITC; Xylene Orange; Y66F; Y66H; Y66W; Yellow GFP (Yellow shifted); Green Fluorescent Protein; YFP (Yellow Fluorescent Protein); YO-PRO-1; YO-PRO-3; YOYO-1; YOYO-3; combinations and derivatives thereof; and the like.

Method of Temporally Modulating an Activity and/or a Phenotype of a Cell

The present disclosure provides methods of temporally modulating the activity and/or phenotype of a cell. The methods comprise: a) at a first time, contacting the composition of the present disclosure with a first enzyme that cleaves a first enzyme cleavage site, wherein said contacting results in removal of a first effector molecule from the target cell; and b) determining the effect of the removal of the first effector molecule on an activity and/or phenotype of the cell. In some cases, the method further comprises: c) at a second time, contacting the composition with a second enzyme that cleaves a second enzyme cleavage site, wherein said contacting results in removal of a second effector molecule from the target cell; and d) determining the effect of the removal of the second effector molecule on an activity and/or phenotype of the cell. In some cases, the method further comprises: e) at a third time, contacting the composition with a third enzyme that cleaves a third enzyme cleavage site, wherein said contacting results in removal of a third effector molecule from the target cell; and f) determining the effect of the removal of the third effector molecule on an activity and/or phenotype of the cell. In some cases, the method further comprises: g) at a fourth time, contacting the composition with a fourth enzyme that cleaves a fourth enzyme cleavage site, wherein said contacting results in removal of a fourth effector molecule from the target cell; and h) determining the effect of the removal of the fourth effector molecule on an activity and/or phenotype of the cell. In some cases, the method further comprises: i) at a fifth time, contacting the composition with a fifth enzyme that cleaves a fifth enzyme cleavage site, wherein said contacting results in removal of a fifth effector molecule from the target cell; and j) determining the effect of the removal of the fifth effector molecule on an activity and/or phenotype of the cell. In some cases, the method further comprises: j) at a sixth time, contacting the composition with a sixth enzyme that cleaves a sixth enzyme cleavage site, wherein said contacting results in removal of a sixth effector molecule from the target cell; and k) determining the effect of the removal of the sixth effector molecule on an activity and/or phenotype of the cell. In some cases, the method further comprises: l) at a seventh time, contacting the composition with a seventh enzyme that cleaves a seventh enzyme cleavage site, wherein said contacting results in removal of a seventh effector molecule from the target cell; and m) determining the effect of the removal of the seventh effector molecule on an activity and/or phenotype of the cell. In some cases, the method further comprises: n) at an eighth time, contacting the composition with an eighth enzyme that cleaves an eighth enzyme cleavage site, wherein said contacting results in removal of an eighth effector molecule from the target cell; and o) determining the effect of the removal of the eighth effector molecule on an activity and/or phenotype of the cell. In some cases, the method further comprises: p) at a ninth time, contacting the composition with a ninth enzyme that cleaves a ninth enzyme cleavage site, wherein said contacting results in removal of a ninth effector molecule from the target cell; and q) determining the effect of the removal of the ninth effector molecule on an activity and/or phenotype of the cell. In some cases, the method further comprises: r) at a tenth time, contacting the composition with a tenth enzyme that cleaves a tenth enzyme cleavage site, wherein said contacting results in removal of a tenth effector molecule from the target cell; and s) determining the effect of the removal of the tenth effector molecule on an activity and/or phenotype of the cell.

Activities of a cell that can be detected following temporal removal of one or more effector molecules include, but are not limited to, proliferation, differentiation, secretion of a cytokine, apoptosis, metabolic activity, cytoskeleton reorganization, migration, and the like. Assays to detect and/or measure such cellular activities are known in the art. For example, methods of detecting and measuring cell proliferation include use of a [³H]-thymidine incorporation assay; a 5-bromo-2′-deoxy-uridine (BrdU) assay, which involves incorporation of BrdU into newly synthesized DNA and detection using an anti-BrdU antibody; an MTT (3-[4,5-dimethylthiazol-2-yl]-2,5-diphenyltetrazolium bromide; thiazolyl blue) assay; an XTT assay (measurement of cell proliferation based upon the reduction of the tetrazolium salt, 2,3-Bis(2-methoxy-4-nitro-5-sulfophenyl)-2H-tetrazolium-5-carboxanilide); a baseclick EdU assay (Salic and Mitchison (2008) Proc. Natd. Acad. Sci. USA 105:2415); a water-soluble tetrazolium salts (WST-1) assay; a luminescent ATP assay; a Ki67 assay (antibodies to nuclear proliferation protein Ki67 are used to measure cellular proliferation); a CFSE (5(6)-carboxyfluorescein diacetate N-succinimidyl ester) assay (CFSE, a non-fluorescent cell permeable dye, is cleaved by intracellular esterases, resulting in green fluorescence); and the like.

Phenotypes of a cell that can be detected following temporal removal of one or more effector molecules include, but are not limited to, expression of a cell surface marker; expression of an intracellular marker; a genomic phenotype; a transcriptomic phenotype; an epigenomic phenotype; and the like. Assays to detect and/or measure various phenotypes are known in the art. For example, cell surface expression of a marker (e.g., cell surface expression of a polypeptide) can be detected and/or measured using a fluorescence activated cell sorting (FACS) method.

As one non-limiting example, the effect of temporal removal of one or more effector molecules on stem cell differentiation can be determined by detecting expression of one or more differentiation markers by the cell.

### Solid Support

The present disclosure provides a solid support comprising one or more patterns exposing an aldehyde-reactive substrate; and one or more tethered oligonucleotides covalently attached to the exposed aldehyde-reactive substrate within the one or more patterns.

The present disclosure provides a solid support comprising: a) one or more patterns exposing an aldehyde-reactive substrate; b) one or more tethered oligonucleotides, covalently attached to the one or more patterns via an amine-modified termini at the 5′ end of the plurality of tethered oligonucleotides, wherein the one or more of tethered oligonucleotides comprises a nucleotide sequence that, when hybridized to a complementary nucleotide sequence present in an untethered oligonucleotide, generates a restriction enzyme cleavage site or a site that is cleavable by a CRISPR/Cas effector polypeptide; and c) one or more untethered oligonucleotides.

A solid support, used in its conventional sense, refers to a surface upon which another element, such as functional groups or molecules, may be adhered. A solid support may be configured as a substrate. Suitable solid supports can have a variety of shapes, sizes, forms, and compositions and can be derived from naturally occurring materials, naturally occurring materials that have been synthetically modified, or synthetic materials. Non-limiting examples of suitable support materials include, but are not limited to, nitrocellulose, glasses, silicas, teflons, metals (for example, gold, platinum, and the like), or other materials. Non-limiting examples of solid support substrates include, but are not limited to polymeric materials, including plastics (for example, polytetrafluoroethylene, polypropylene, polystyrene, polycarbonate, and blends thereof, and the like), polysaccharides such as agarose and dextran, polyacrylamides, polystyrenes, polyvinyl alcohols, copolymers of hydroxyethyl methacrylate and methyl methacrylate, and the like. A solid support may be homogenous or a composite structure of two or more different materials, e.g., where the solid support includes a first base material that is coated on a surface with one or more additional different coating materials.

In some cases, the solid support is a glass substrate (e.g., a glass slide). In some cases, the surface of the solid support is patterned with aldehyde groups to create one or more patterns exposing the aldehyde-functionalized substrate. The one or more patterns exposing the aldehyde-reactive substrate provides a spatial platform for the attachment of molecules. These groups can capture molecules through physical attraction, such as electrostatic interaction, for example, or chemical binding.

In some cases, the one or more patterns exposing the aldehyde-reactive substrates comprises a diameter ranging from 50 nm to 50 mm. In some cases, the one or more patterns exposing the aldehyde-reactive substrates comprises a diameter ranging from 50 nm to 100 nm. In some cases, the one or more patterns exposing the aldehyde-reactive substrates comprises a diameter ranging from 50 nm to 100 μm. In some cases, the one or more patterns exposing the aldehyde-reactive substrates comprises a diameter ranging from 2 to 5 μm, 5 to 10 μm, 10 to 15 μm, 15 to 20 μm, 20 to 25 μm, 25 to 30 μm, 30 to 35 μm, 35 to 40 μm, 40 to 45 μm, or 45 to 50 μm. In some cases, the one or more patterns exposing the aldehyde-reactive substrates comprises a diameter ranging from 50 to 100 μm, 100 to 150 μm, 150 to 200 μm, 200 to 250 μm, 250 to 300 μm, 300 to 350 μm, 350 to 400 μm, 400 to 450 μm, or 450 to 500 μm. In some cases, the one or more patterns exposing the aldehyde-reactive substrates comprises a diameter ranging from 1 mm to 5 mm, 5 mm to 10 mm, 10 mm to 15 mm, 15 mm to 20 mm, 20 mm to 25 mm, or 25 mm to 30 mm.

In some cases, the one or more patterns exposing the aldehyde-reactive substrate is a cylindrical shape, a circular shape, a square shape, a spherical shape, a cylindrical shape, a rectangular shape, or a combination thereof. In some cases, the one or more patterns comprises one or more micro-islands. The one or more patterns exposing the aldehyde-reactive substrate is not limited to the shapes and/or sizes as described herein and can be any shape and/or size as required per conditions specific to its intended use.

In some cases, the solid support comprising the one or more patterns exposing the aldehyde-reactive substrate comprises one or more fiducial markers. In some cases, the fiducial markers are metal alignment markers. In some cases, the alignment markers on the solid support substrate provides for registration of the one or more patterns exposing the aldehyde-reactive substrate.

In some cases, the aldehyde-reactive substrate comprises a grid surrounding the one or more patterns. In some cases, the grid separates two or more patterns, three or more patterns, four or more patterns, five or more patterns, six or more patterns, seven or more patterns, eight or more patterns, nine or more patterns, or ten or more from one another. In some cases, the grid creates one or more micro-islands separated from one another, each comprising one or more patterns. In some cases, each of the micro-islands are separated from one another by a distance ranging from about 50 μm to about 500 μm. In some cases, each of the micro-islands are separated from one another by a distance ranging from about 50-100 μm, 100-150 μm, 150-200 μm, 200-250 μm, 250-300 μm, 300-350 μm, 350-400 μm, 400-450 μm, or 450-500 μm. In some cases, each of the micro-islands are separated from one another by a distance ranging from about 1-2 mm, 2-4 mm, 4-6 mm, 6-8 mm, 8-10 mm, 10-12 mm, 12-14 mm, 14-16 mm, 16-18 mm, or 18-20 mm. In some cases, the one or more microislands is a cylindrical shape, a circular shape, a square shape, a spherical shape, a cylindrical shape, a rectangular shape, or a combination thereof. In some cases, the one or more microislands comprises a length ranging from 1 μm to 500 μm and a width ranging from 1 μm to 500. In some cases, the one or more microislands comprises a length of 141 μm and a width of 141 μm. In some cases, the one or more microislands comprises a length ranging from 1 mm to 50 mm and a width ranging from 1 mm to 50 mm. The one or more microislands comprising one or more patterns exposing the aldehyde-reactive substrate is not limited to the shapes and/or sizes as described herein and can be any shape and/or size as required per conditions specific to its intended use.

In some cases, the one or more patterns comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, 500 or more, 1000 or more, 1500 or more, or 2000 or more patterns.

In some cases, the aldehyde-reactive substrate comprises a grid surrounding the one or more patterns. In some cases, the grid separates two or more array of patterns, three or more array of patterns, four or more array of patterns, five or more array of patterns, six or more array of patterns, seven or more array of patterns, eight or more array of patterns, nine or more array of patterns, or ten or more array of patterns separated from one another. In some cases, an array of patterns comprises 2 or more, 6 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, or 500 or more patterns exposing the aldehyde-reactive substrate. In some cases, the grid separates a first array of patterns exposing the aldehyde-reactive substrate from a second array of patterns exposing the aldehyde-reactive substrate. In some cases, the aldehyde-reactive substrate comprises a 2×2 array of patterns, a 3×3 array of patterns, a 4×4 array of patterns, a 5×5 array of patterns, a 6×6 array of patterns, a 7×7 array of patterns, an 8×8 array of patterns, a 9×9 array of patterns, or a 10×10 array of patterns. In some cases, the aldehyde-reactive substrate comprises a 15×15 array of patterns, a 20×20 array of patterns, a 25×25 array of patterns, 50×50 array of patterns, 100×100 array of patterns, 150×150 array of patterns, or more than 150×150 array of patterns. In some cases, the grid creates one or more micro-islands separated from one another, each comprising an array of patterns. In some cases, the one or more microislands is a cylindrical shape, a circular shape, a square shape, a spherical shape, a cylindrical shape, a rectangular shape, or a combination thereof. In some cases, the one or more microislands comprises a length ranging from 1 μm to 500 μm and a width ranging from 1 μm to 500. In some cases, the one or more microislands comprises a length of 141 μm and a width of 141 μm. In some cases, the one or more microislands comprises a length ranging from 1 mm to 50 mm and a width ranging from 1 mm to 50 mm. The one or more microislands comprising one or more patterns exposing the aldehyde-reactive substrate is not limited to the shapes and/or sizes as described herein and can be any shape and/or size as required per conditions specific to its intended use.

The grid can be made of any suitable material, such as, but not limited to, plastics (for example, polytetrafluoroethylene, polypropylene, polystyrene, polycarbonate, and blends thereof, and the like), polysaccharides such as agarose and dextran, polyacrylamides, polystyrenes, polyvinyl alcohols, copolymers of hydroxyethyl methacrylate and methyl methacrylate, and the like. The grid can be made using any known photolithographic or electron beam lithography technique.

Aspects of the present disclosure include one or more tethered oligonucleotides attached to the solid support in a patterned array. In some cases, the one or more tethered oligonucleotides is a 5′ amine-modified oligonucleotide. In some cases, the one or more tethered oligonucleotides is covalently attached to the exposed aldehyde on the solid support substrate. For example, the 5′ end of one or more tethered oligonucleotides react with the exposed aldehyde groups within the one or more patterns to induce the formation of Schiff bonds (C═N) between the terminal amine on the one or more tethered oligonucleotide and the exposed aldehyde on the solid support. The one or more tethered oligonucleotides are then covalently attached to the exposed surface of the aldehyde groups by converting hydrolysable Schiff base bonds to single C—N bonds.

In some cases, the one or more tethered oligonucleotides are covalently attached to the exposed aldehyde-reactive substrate within the one or more patterns, thereby forming a tethered DNA patterned substrate. The tethered DNA pattern comprises a diameter ranging from 50 nm to 50 mm. In some cases, the one or more tethered DNA patterns comprises a diameter ranging from 50 nm to 100 nm. In some cases, the one or more Tethered DNA patterns comprises a diameter ranging from 50 nm to 100 μm. In some cases, the one or more tethered DNA patterns comprises a diameter ranging from 2 to 5 μm, 5 to 10 μm, 10 to 15 μm, 15 to 20 μm, 20 to 25 μm, 25 to 30 μm, 30 to 35 μm, 35 to 40 μm, 40 to 45 μm, or 45 to 50 μm. In some cases, the one or more tethered DNA patterns comprises a diameter ranging from 50 to 100 μm, 100 to 150 μm, 150 to 200 μm, 200 to 250 μm, 250 to 300 μm, 300 to 350 μm, 350 to 400 μm, 400 to 450 μm, or 450 to 500 μm. In some cases, the one or more tethered DNA patterns comprises a diameter ranging from 1 mm to 5 mm, 5 mm to 10 mm, 10 mm to 15 mm, 15 mm to 20 mm, 20 mm to 25 mm, or 25 mm to 30 mm.

In some cases, the one or more tethered DNA patterns is a cylindrical shape, a circular shape, a square shape, a spherical shape, a cylindrical shape, a rectangular shape, or a combination thereof. In some cases, the one or more tethered DNA patterns comprises one or more micro-islands. In some cases, the one or more tethered DNA patterns comprises one or more tethered DNA patterns selected from the tethered DNA patterns as shown in FIG. 26. The one or more tethered DNA patterns is not limited to the shapes and/or sizes as described herein and can be any shape and/or size as required per conditions specific to its intended use.

In some cases, the one or more DNA patterns comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, 500 or more, 1000 or more, 1500 or more, or 2000 or more tethered DNA patterns.

In some cases, the DNA pattern comprises two or more array of tethered DNA patterns, three or more array of tethered DNA patterns, four or more array of tethered DNA patterns, five or more array of tethered DNA patterns, six or more array of tethered DNA patterns, seven or more array of tethered DNA patterns, eight or more array of tethered DNA patterns, nine or more array of tethered DNA patterns, or ten or more array of tethered DNA patterns separated from one another. In some cases, an array of tethered DNA patterns comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, or 500 or more tethered DNA patterns. In some cases, the grid separates a first array of tethered DNA patterns from a second array of tethered DNA patterns. In some cases, the aldehyde-reactive substrate comprises a 2×2 array of tethered DNA patterns, a 3×3 array of tethered DNA patterns, a 4×4 array of tethered DNA patterns, a 5×5 array of tethered DNA patterns, a 6×6 array of tethered DNA patterns, a 7×7 array of DNA patterns, an 8×8 array of tethered DNA patterns, a 9×9 array of tethered DNA patterns, or a 10×10 array of tethered DNA patterns. In some cases, the aldehyde-reactive substrate comprises a 15×15 array of tethered DNA patterns, a 20×20 array of tethered DNA patterns, or a 25×25 array of tethered DNA patterns. In some cases, the grid creates one or more micro-islands separated from one another, each comprising an array of tethered DNA patterns. In some cases, the one or more microislands is a cylindrical shape, a circular shape, a square shape, a spherical shape, a cylindrical shape, a rectangular shape, or a combination thereof. In some cases, the one or more microislands comprises a length ranging from 1 μm to 500 μm and a width ranging from 1 μm to 500 μm. In some cases, the one or more microislands comprises a length of 141 μm and a width of 141 μm. In some cases, the one or more microislands comprises a length ranging from 1 mm to 50 mm and a width ranging from 1 mm to 50 mm. In some cases, the one or more microislands comprises one or more microislands selected from the microislands as shown in FIG. 26. The one or more microislands comprising one or more tethered DNA patterns is not limited to the shapes and/or sizes as described herein and can be any shape and/or size as required per conditions specific to its intended use.

In some cases, the one or more tethered oligonucleotides comprises a nucleotide sequence that, when hybridized to a complementary nucleotide sequence present in an untethered oligonucleotide, generates an enzyme cleavage site. In some cases, the one or more tethered oligonucleotides comprising a nucleotide sequence that, when hybridized to a complementary nucleotide sequence present in an untethered oligonucleotide, directs the spatial organization of the hybridized untethered oligonucleotide on the solid support. In some cases, the one or more patterns comprising the hybridized untethered oligonucleotide provides for spatial heterogeneity on the solid support. In some cases, the one or more tethered oligonucleotides are positioned orthogonally relative to the aldehyde-reactive substrate.

The tethered and the untethered oligonucleotides have a length of from about 15 nucleotides (nt) to about 50 nt; e.g., the tethered and the untethered oligonucleotides have a length of from about 15 nt to about 20 nt (e.g., 15 nt, 16 nt, 17 nt, 18 nt, 19 nt, or 20 nt), from about 20 nt to about 25 nt (e.g., 20 nt, 21 nt, 22 nt, 23 nt, 24 nt, or 25 nt), from about 25 nt to about 30 nt (e.g., 25 nt, 26 nt, 27 nt, 28 nt, 29 nt, or 30 nt), from about 30 nt to about 35 nt, from about 35 nt to about 40 nt, from about 40 nt to about 45 nt, or from about 45 nt to about 50 nt.

As noted above, each of the one or more tethered oligonucleotides comprises a nucleotide sequence that, when hybridized to a complementary nucleotide sequence present in an untethered oligonucleotide, generates an enzyme cleavage site. Such a nucleotide sequence is referred to herein as a “hybridization nucleotide sequence.” The length of the hybridization nucleotide sequence can be from about 6 nucleotides (nt) to about 30 nt; e.g., the length of the hybridization nucleotide sequence can be from about 6 nt to about 10 nt (e.g., 6 nt, 7 nt, 8 nt, 9 nt, or 10 nt), from about 10 nt to about 15 nt (e.g., 10 nt, 11 nt, 12 nt, 13 nt, 14 nt, or 15 nt), from about 15 nt to about 20 nt (e.g., 15 nt, 16 nt, 17 nt, 18 nt, 19 nt, or 20 nt), from about 20 nt to about 25 nt, or from about 25 nt to about 30 nt.

In some cases, the solid support comprises one or more untethered oligonucleotides. In some cases, the one or more untethered oligonucleotides comprises a nucleotide sequence that hybridizes to the one or more tethered oligonucleotides in a patterned array. In some cases, hybridization of an untethered oligonucleotide to a tethered oligonucleotide generates an enzyme cleavage site, which allows for temporally controlled removal of an effector molecule. In some cases, the untethered oligonucleotides each comprises: i) the nucleotide sequence that generates an enzyme cleavage site (e.g., where the enzyme cleavage site is a restriction enzyme cleavage site or a site that is cleavable by a CRISPR/Cas effector polypeptide); ii) either a cell, or an effector molecule that affects an activity and/or a phenotype of a cell, where the cell or the effector molecule is attached to the untethered oligonucleotide at the 5′ end of the untethered oligonucleotides. In some cases, the untethered oligonucleotides each include, attached thereto, a fluorophore. In some cases, the fluorophore is attached to the untethered oligonucleotide at the 3′ end of the untethered oligonucleotide.

In some cases, the one or more untethered oligonucleotides comprising a nucleotide sequence that hybridizes to the complementary nucleotide sequence in one or more tethered oligonucleotides and form a patterned array of hybridized untethered oligonucleotides on the solid support. In some cases, the patterned array of hybridized untethered oligonucleotides forms the same patterned array as the tethered oligonucleotides covalently attached to the one or more patterns on the solid support exposing an aldehyde-reactive substrate. In some cases, the one or more patterns comprising the hybridized untethered oligonucleotide provides for spatial heterogeneity on the solid support. In some cases, the one or more untethered oligonucleotides hybridized to the one or more tethered oligonucleotides are positioned orthogonally relative to the aldehyde-reactive substrate.

In some cases, the one or more untethered oligonucleotides comprises at least a first, a second, and a third plurality of hybridized, untethered oligonucleotides that are bound to the tethered oligonucleotides in the patterned array (e.g., via hybridization of the tethered oligonucleotides on the solid support). In some cases, the first plurality of hybridized, untethered oligonucleotides comprises, bound to the 5′ end of the oligonucleotides, a first effector molecule, wherein the first plurality of hybridized, untethered oligonucleotides generates a first enzyme cleavage site. In some cases, the second plurality of hybridized, untethered oligonucleotides comprises, bound to the 5′ end of the oligonucleotides, a second effector molecule, wherein the second plurality of hybridized, untethered oligonucleotides generates a second enzyme cleavage site. In some cases, the third plurality of hybridized, untethered oligonucleotides comprises, bound to the 5′ end of the oligonucleotides, a target cell. In some cases, the one or more untethered oligonucleotides further comprises fourth plurality of hybridized, untethered oligonucleotides, wherein the fourth plurality of hybridized, untethered oligonucleotides bound to the 5′ end of the oligonucleotides, a third effector molecule, wherein the fourth plurality of hybridized, untethered oligonucleotides generates a third enzyme cleavage site.

Non-limiting examples of untethered oligonucleotides comprising a nucleotide sequence includes, but is not limited to: A′-BamHI-Cy3: [Cy3] CAGTCAGTCAGTCAGTCAGT (SEQ ID NO:1); A′-Cy5: [Cy5] CAGTCAGTCAGTCAGTCAGT (SEQ ID NO:2); F′-AlexaFluor 488: [AlexaFluor488] TTCTTCTTCGTTCTTCTTCT (SEQ ID NO:3); F′-Lipid: [Lipid GTAACGATCCAGCTGTCACTTTTTTTTTTTTTTTTTTTTTTTTTTTTTTTTTTTTTTTTTTTTTTT TTTTTTTTTTTTTTTTCTTCTTCGTTCTTCTTCT(SEQ ID NO:4); Azide-G′-Cy3: [Azide] CTCTCTCTCTCTCTCTGGCT [Cy3] (SEQ ID NO:5); G′-Cy5: [Cy5] CTCTCTCTCTCTCTCTGGCT (SEQ ID NO:6); G′-Cy3: [Cy3] CTCTCTCTCTCTCTCTGGCT (SEQ ID NO:7); G-EcoRI-NH₂: [AminoC6] AGCCAGAGAGAGAGAGAGAGCTAAGC AGAATTCCCATAAG (SEQ ID NO:8); G′-EcoRI-Cy5: [Cy5] CAGCTGGATCGTTACCCACCCTAGTCATTGGAGGTGACGAGTGAGTCGTATGA (SEQ ID NO:9); and CoAnchor Lipid: [Lipid] AGTGACAGCTGGATCGTTAC (SEQ ID NO:10).

In some cases, the solid support comprises one or more flow cells. In some cases, the one or more flow cells comprises a flow cell chamber. In some cases, the flow cell chamber is configured to flow the untethered oligonucleotides of the present disclosure across the surface of the flow cell chamber. In some cases, the flow cell comprises an inlet. In some cases, the flow cell comprises an outlet.

In some cases, the one or more flow cells are positioned over the one or more patterns exposing the aldehyde-reactive substrate. In some cases, the one or more flow cells are positioned over the one or more patterns exposing the aldehyde-reactive substrate. In some cases, the one or more flow cells are positioned over a microisland surrounding the one or more patterns, or an more array of patterns, exposing the aldehyde-reactive substrate. In some cases, the one or more flow cells are affixed to the solid support substrate such that each flow cell is positioned over the one or more microislands comprising one or more patterns, or an array of patterns. In some cases, the one or more patterns, or an array of patterns is contained within the flow cell chamber. In some cases, the one or more flow cells are made from a polymeric material. Non-limiting examples of polymeric materials include, but are not limited to, plastics (for example, polytetrafluoroethylene, polypropylene, polystyrene, polycarbonate, and blends thereof, and the like), PDMS, polysaccharides such as agarose and dextran, polyacrylamides, polystyrenes, polyvinyl alcohols, copolymers of hydroxyethyl methacrylate and methyl methacrylate, and the like.

In some cases, the one or more flow cells is configured to flow one or more untethered oligonucleotides across the solid support comprising the one or more tethered oligonucleotides. For example, the one or more flow cells concentrates the cells to the surface of the solid support and increases the probability of hybridization between the untethered oligonucleotides and the tethered oligonucleotides.

### Method of Making a DNA Patterned Surface

Aspects of the present disclosure include methods of making a DNA patterned surface, wherein the method comprises a) functionalizing a surface of a solid support with aldehyde groups to form an aldehyde-reactive substrate; b) applying a cell-resistive layer onto the aldehyde-reactive substrate; c) heating the cell-resistive layer; d) applying a mask comprising one or more patterns to the cell-resistive layer; e) exposing the aldehyde-reactive substrate, the cell-resistive layer, and the mask to ultraviolet (UV) light to create one or more patterns exposing the aldehyde-reactive substrate; f) flowing one or more tethered oligonucleotides over the one or more patterns exposing the aldehyde-reactive substrate, wherein the one or more tethered oligonucleotides comprises a nucleotide sequence that, when hybridized to a complementary nucleotide sequence present in an untethered oligonucleotide, generates an enzyme cleavage site; g) conjugating the 5′ amine-modified end of the one or more tethered oligonucleotides to the one or more patterns exposing the aldehyde-reactive substrate; and h) removing the cell-resistive layer. Upon removal of the cell-resistive layer, a DNA patterned layer is formed. The DNA patterned layer comprises tethered oligonucleotides covalently attached to the patterned aldehyde-reactive substrate.

In some cases, the method of making a DNA patterned surface further comprises repeating steps a) through h) to form two or more DNA patterned layers, three or more DNA patterned layers, four or more DNA patterned layers, five or more DNA patterned layers, five or more DNA patterned layers, six or more DNA patterned layers, seven or more DNA patterned layers, eight or more DNA patterned layers, nine or more DNA patterned layers, or ten or more DNA patterned layers. In some cases, repeating steps a) through h) forms a second DNA patterned layer. In some cases, after repeating steps a) through h) to form a second DNA patterned layer, the method is repeated for a third time to form a third DNA patterned layer. In some cases, after repeating steps a) through h) to form a third DNA patterned layer, the method is repeated for a fourth time to form a fourth DNA patterned layer. In some cases, after repeating steps a) through h) to form a fourth DNA patterned layer, the method is repeated for a fifth time to form a fifth DNA patterned layer. In some cases, after repeating steps a) through h) to form a fifth DNA patterned layer, the method is repeated for a sixth time to form a sixth DNA patterned layer. In some cases, after repeating steps a) through h) to form a sixth DNA patterned layer, the method is repeated for a seventh time to form a seventh DNA patterned layer. In some cases, after repeating steps a) through h) to form a seventh DNA patterned layer, the method is repeated for an eighth time to form an eighth DNA patterned layer. In some cases, after repeating steps a) through h) to form an eighth DNA patterned layer, the method is repeated for a ninth time to form a ninth DNA patterned layer. In some cases, after repeating steps a) through h) to form a ninth DNA patterned layer, the method is repeated for a tenth time to form a tenth DNA patterned layer. Steps a) through h) can repeated up to one or more times, two or more times, three or more times, four or more times, five or more times, six or more times, seven or more times, eight or more times, nine or more times, ten or more times, 11 or more times, 12 or more times, 13 or more times, 14 or more times, or 15 or more times without losing any functionality of the tethered oligonucleotides on the aldehyde-reactive substrate.

In some cases, each patterned layer comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, 500 or more, 1000 or more, 1500 or more, or 2000 or more tethered DNA patterns.

A solid support, used in its conventional sense, refers to a surface upon which another element, such as functional groups or molecules, may be adhered. A solid support may be configured as a substrate. Suitable solid supports can have a variety of shapes, sizes, forms, and compositions and can be derived from naturally occurring materials, naturally occurring materials that have been synthetically modified, or synthetic materials. Non-limiting examples of suitable support materials include, but are not limited to, nitrocellulose, glasses, silicas, teflons, metals (for example, gold, platinum, and the like), or other materials. Non-limiting examples of solid support substrates include polymeric materials, which include, but are not limited to plastics (for example, polytetrafluoroethylene, polypropylene, polystyrene, polycarbonate, and blends thereof, and the like), polydimethylsiloxane (PDMS), polysaccharides such as agarose and dextran, polyacrylamides, polystyrenes, polyvinyl alcohols, copolymers of hydroxyethyl methacrylate and methyl methacrylate, and the like. A solid support may be homogenous or a composite structure of two or more different materials, e.g., where the solid support includes a first base material that is coated on a surface with one or more additional different coating materials.

In some cases, the solid support is a glass substrate (e.g., a glass slide). In some cases, the method includes coating the solid support with aldehyde groups to create an aldehyde-functionalized surface of the solid support. The aldehyde functionalized surface is an aldehyde-reactive surface. Aldehyde-functional groups coated on a surface have can provide a platform for attachment of molecules. These groups can capture molecules through physical attraction, such as electrostatic interaction, for example, or chemical binding. In some cases, the method comprises coating the solid support with aldehyde-functional groups by covalently attaching the aldehyde-functional groups to the surface of the solid support. Aldehyde-groups can be functionalized to the solid support using any known technique.

In some cases, the solid support comprises applying a cell-resistive layer on the aldehyde-reactive substrate. In some cases, applying a cell-resistive layer on the aldehyde-reactive substrate comprises dipping the aldehyde-reactive substrate in a polymer layer containing the cell-resist, pipetting the cell-resist solution on to the aldehyde-reactive substrate, or by spin coating a cell-resistive layer onto the aldehyde-reactive substrate. For example, in some cases, a liquid resist is dropped onto the solid support, which is then spin-coated on the surface of the substrate to form a coating. In some cases, the method comprises spinning a cell-resistive layer at 1000 to 6000 rpm. In some cases, the method comprises spinning a cell-resistive layer at 1000, 2000, 3000, 4000, 5000, or 6000 rpm. In some cases, the method comprises spinning a cell-resistive layer at 3000 rpm. In some cases, the method comprises spinning the cell-resistive layer for a period of time of from about 1 second to about 5 minutes. In some cases, the method comprises spinning the cell-resistive layer for a period of time of from about 1 second to about 30 seconds, from about 30 seconds to about 60 seconds, from about 60 seconds to about 90 seconds, from about 90 seconds to about 120 seconds, from about 120 seconds to about 150 seconds, from about 150 seconds to about 180 seconds, from about 180 seconds to about 210 seconds, or from about 210 seconds to about 240 seconds.

In some cases, the method comprises heating the cell-resistive layer. In some cases, heating the cell-resistive layer comprises soft-baking the cell-resistive layer using known lithographic techniques. In some cases, soft-baking the cell-resistive layer comprises heating the cell resistive layer for a time period of about 30 seconds or more, about 60 seconds or more, about 90 seconds or more, about 120 seconds or more, about 150 seconds or more, about 180 seconds or more, or about 210 seconds or more. In some cases, the cell-resistive layer is heated at a temperature of about 50° C. or more, about 60° C. or more, about 70° C. or more, about 80° C. or more, about 90° C. or more, about 100° C. or more, about 110° C. or more, or about 120° C. or more.

In some cases, the cell-resistive layer is a photoresist, an electron beam resist, an ion beam resist, or any known cell-resist used in lithographic techniques used to define the dimensions and shape (e.g. spatial configuration of the patterns) exposing the aldehyde-reactive substrate to which tethered oligonucleotides can attach to. A photoresist, as used herein and in its conventional sense, is a light-sensitive material used in photolithography to create a patterned coating on a surface. In some cases, photoresists are composed of adhesive agents, sensitizers, and solvents. In some cases, the photoresist is a positive photoresist. In some cases, the cell-resistive layer is a S1813 photoresist. A mask that defines the shape and surface area of one or more patterns may be positioned over the photoresist. In some cases, the mask allows light to pass through the one or more patterned regions in the mask, thereby exposing the one or more patterned regions of the positive photoresist to light and making the photoresist in the one or more patterned regions soluble to the photoresist developer. Accordingly, upon development of the photoresist, the one or more patterned region of the photoresist is removed.

In some cases, the photoresist is a negative photoresist. In these cases, the mask may be designed to allow light to pass through one or more patterned regions in the mask, thereby exposing one or more patterned regions of the negative photoresist to light and making the photoresist in the regions surrounding the one or more patterned regions soluble to the photoresist developer. Accordingly, upon development of the photoresist, the regions surrounding the one or more patterned regions of the photoresist is removed.

A variety of positive and negative photoresists may be used in the methods disclosed herein. As used herein, the phrase “positive photoresist” refers to a type of photoresist in which the portion of the photoresist that is exposed to light becomes soluble to the photoresist developer. While, the portion of the photoresist that is unexposed remains insoluble to the photoresist developer. As used herein, the phrase “negative photoresist” refers to a type of photoresist in which the portion of the photoresist that is exposed to light becomes insoluble to the photoresist developer. While, the unexposed portion of the photoresist is dissolved by the photoresist developer. Non-limiting examples of photoresist include, but are not limited to, Hoechst AZ 4620, Hoechst AZ 4562, AZ 1500, e.g., AZ 1514 H, Shipley 1813, Shipley 1400-17, Shipley 1400-27, Shipley 1400-37, etc.

In some cases, a cell-resistive layer is an electron beam resist. In some cases, an electron beam resist is an electron sensitive material used in electron beam lithography to create a patterned coating on a surface. In some cases, the electron beam resist comprises a polymer dissolved in a liquid solvent.

In some cases, the method of the present disclosure comprises applying a mask comprising one or more patterns to the cell-resistive layer. In some cases, a mask defines the pattern exposing the aldehyde-reactive substrate. Masks may be generated by standard procedure based on the desired pattern exposing the aldehyde-reactive substrate. In some cases, the mask comprises one or more shapes and/or dimensions that allow radiation (e.g. light, ions, electrons, etc.) to pass through in a defined pattern. In some cases, the mask is a plate or film with transparent areas to allow radiation to pass through. In some cases, the mask is an optical mask. In some cases, the mask is a digital mask. In some cases, the optical mask is used in electron beam lithography, where a focused beam of electrons is scanned on the solid support substrate such that patterns are created and transmitted electronically.

For example, in photolithography, one or more patterns in the mask provides for exposure of the one or more patterns to light to solubilize the cell-resistive layers within the location of the one or more patterns, thereby creating one or more patterns in the cell-resistive layer. In some cases, light may be used to expose a defined region of a photoresist layer via the mask. In certain cases, light may be a short wavelength light (for example, a wavelength of about 100 nm-440 nm), such as, ultra violet (UV) light, deep UV light, H and I lines of a mercury-vapor lamp. In some cases, the light is emitted at a wavelength of 193 nm, 248 nm, or 365 nm. The step of exposing the photoresist to light may be followed with a step of photoresist development where the photoresist is contacted with a photoresist developer. In embodiments, where a positive photoresist is used, the regions of the positive photoresist layer exposed to light are washed away in the photoresist developer. In embodiments, where a negative photoresist is used, the regions of the negative photoresist layer not exposed to light are washed away in the photoresist developer.

Any standard photoresist developer compatible with the photoresist deposited may be used in the methods described herein. As such, a positive developer may be used to remove any positive photoresist exposed to light. In certain cases, a negative developer may be used to remove any negative photoresist not exposed to light. In some cases, the photoresist is washed with an MF-321 developer. In some cases, after washing away the photoresist layer in the photoresist developer, the one or more patterns exposing the aldehyde-reactive substrate is washed with deionized water and dried with nitrogen gas.

In some cases, the cell-resistive layer comprises one or more patterns exposing the aldehyde-reactive substrate. In some cases, the one or more patterns exposing the aldehyde-reactive substrates comprises a diameter ranging from 50 nm to 50 mm. In some cases, the one or more patterns exposing the aldehyde-reactive substrates comprises a diameter ranging from 50 nm to 100 nm. In some cases, the one or more patterns exposing the aldehyde-reactive substrates comprises a diameter ranging from 50 nm to 500 μm. In some cases, the one or more patterns exposing the aldehyde-reactive substrates comprises a diameter ranging from 2 to 5 μm, 5 to 10 μm, 10 to 15 μm, 15 to 20 μm, 20 to 25 μm, 25 to 30 μm, 30 to 35 μm, 35 to 40 μm, 40 to 45 μm, or 45 to 50 μm. In some cases, the one or more patterns exposing the aldehyde-reactive substrates comprises a diameter ranging from 50 to 100 μm, 100 to 150 μm, 150 to 200 μm, 200 to 250 μm, 250 to 300 μm, 300 to 350 μm, 350 to 400 μm, 400 to 450 μm, or 450 to 500 μm. In some cases, the one or more patterns exposing the aldehyde-reactive substrates comprises a diameter ranging from 1 mm to 5 mm, 5 mm to 10 mm, 10 mm to 15 mm, 15 mm to 20 mm, 20 mm to 25 mm, or 25 mm to 30 mm.

In some cases, the one or more patterns of the cell-resistive layer exposing the aldehyde-reactive substrate is a cylindrical shaped pattern, a circular shaped pattern, a square shaped pattern, a spherical shaped pattern, a cylindrical shaped pattern, or a rectangular shaped pattern. In some cases, the one or more patterns of the cell-resistive layer exposing the aldehyde-reactive substrate is a shaped pattern that mimics a biological structure. In some cases, the one or more patterns of the cell-resistive layer exposing the aldehyde-reactive substrate is shaped as any type of biological tissue. Non-limiting examples of shapes of patterns that mimic a biological structure include, but are not limited to, intestinal folds and/or liver lobules. The cell-resistive layer with one or more patterns is not limited to the shapes and/or sizes as described herein and can be any shape and/or size as required per conditions specific to its intended use.

In some cases, the mask comprising one or more patterns defines the one or more DNA patterns covalently attached to the aldehyde-reactive substrate. In some cases, the shape of the one or more patterns in the mask is a cylindrical shape, a circular shape, a square shape, a spherical shape, a cylindrical shape, or a rectangular shape. In some cases, the one or more patterns on the mask is a shaped pattern that mimics a biological structure. In some cases, the one or more patterns of the cell-resistive layer exposing the aldehyde-reactive substrate is shaped as any type of biological tissue. Non-limiting examples of shapes of patterns that mimic a biological structure include, but are not limited to, intestinal folds and/or liver lobules. The one or more patterns one the mask is not limited to the shapes and/or sizes as described herein and can be any shape and/or size as required per conditions specific to its intended use.

In some cases, the cell-resistive layer with one or more patterns further comprises one or more fiducial markers using standard photolithography techniques. For example, a cell resistive-layer can be patterned using a mask aligner, followed by deposition of a 100 Å thin film of titanium via electron-gun evaporation. The cell-resistive layer and excess metal is removed by acetone lift-off.

Aspects of the present disclosure include flowing one or more tethered oligonucleotides over the one or more patterns exposing the aldehyde-reactive substrate, wherein the one or more tethered oligonucleotides comprises a nucleotide sequence that, when hybridized to a complementary nucleotide sequence present in an untethered oligonucleotide, generates an enzyme cleavage site. In some cases, flowing one or more tethered oligonucleotides over the one or more patterns exposing the aldehyde-reactive substrate comprises dropcasting a solution containing the one or more tethered oligonucleotides over the one or more patterns exposing the aldehyde-reactive substrate. In some cases, the solution containing the one or more tethered oligonucleotides comprises a tethered oligonucleotide concentration ranging from 0.1-0.5 μM, 0.5-5 μM, 5-10 μM, 10-15 μM, 15-20 μM, 20-25 μM, 25-30 μM, 35-40 μM, 45-50, 50-55 μM, 55-60 μM, 60-65 μM, 65-70 μM, 70-75 μM, 75-80 μM, 80-85 μM, 85-90 μM, 90-95 μM, or 95-100 μM. In some cases, the solution containing the one or more tethered oligonucleotides comprises a tethered oligonucleotide concentration of about 0.5 μM, about 1 μM, about 1.5 μM, about 2.0 μM, about 2.5 μM, about 5 μM, about 10 μM, about 20 μM, about 25 μM, about 50 μM, or about 100 μM.

In some cases, the method further comprises, before flowing the one or more tethered oligonucleotides over the one or more patterns exposing the aldehyde-reactive substrate, diluting the tethered oligonucleotides in a buffer. In some cases, the buffer is water. In some cases, the buffer is a salt buffer. In some cases, the buffer is a sodium phosphate buffer. In some cases, the buffer is a saline-sodium citrate (SSC) buffer. In some cases, the buffer incorporates a low salt concentration and preserves the integrity of the patterned cell-resistive layer (e.g. photoresist). In some cases, the pH of the tethered oligonucleotide solution ranges from 8-8.5. In some cases, the pH of the tethered oligonucleotide solution is 8.0. In some cases, the pH of the tethered oligonucleotide solution is 8.5. In some cases, the concentration of the buffer is about 25 mM, about 30 mM, about 35 mM, about 40 mM, about 45 mM, about 50 mM, about 55 mM or about 60 mM.

In some cases, following flowing of the one or more tethered oligonucleotides in a solution, the method comprises covering the solid support to prevent evaporation of the tethered oligonucleotide solution. In some cases, the method further comprises, before conjugating the 5′ amine-modified end of the one or more tethered oligonucleotides to the one or more patterns exposing the aldehyde-reactive substrate, incubating the one or more tethered oligonucleotides on the aldehyde-reactive substrate for a period of time of about 1 minute, 2 minutes, about 3 minutes, about 4 minutes, about 5 minutes, about 6 minutes, about 7 minutes, about 8 minutes, about 9 minutes, or about 10 minutes.

In some cases, the method further comprises heating the one or more tethered oligonucleotide solution on the aldehyde-reactive substrate for period of time of about 30 minutes, or 60 minutes. In some cases, heating comprises placing the aldehyde-reactive substrate in a 75° C. oven to induce the formation of Schiff bonds (C═N) between the terminal amine on the tethered oligonucleotide and the aldehyde on the surface of the solid support substrate.

In some cases, the method further comprises submerging the aldehyde-reactive surface with an aqueous solution (e.g. an aqueous solution that does not degrade the tethered oligonucleotides, the cell-resistive layer, and/or cell viability and fitness). In some cases, the method further comprises submerging the aldehyde-reactive surface with sodium dodecyl sulfate (SDS) in deionized water. In some cases, the method further comprises submerging the aldehyde-reactive surface with sodium dodecyl sulfate (SDS) in deionized water. In some cases, the aldehyde-reactive surface is submerged in 0.4% SDS in deionized water. In some cases, the method further comprises rinsing the aldehyde-reactive substrate with deionized water to remove excess oligonucleotides.

In some cases, the method further comprises covalently conjugating the tethered oligonucleotides to the one or more patterns exposing the aldehyde-reactive substrate. In some cases, covalently conjugating the tethered oligonucleotides to the one or more patterns exposing the aldehyde-reactive substrates converts the hydrolysable Schiff base to single C—N bonds. In some cases, converting the hydrolysable Schiff base to single C—N bonds comprises reductive amine-condensation. In some cases, reductive-amine condensation comprises heating the aldehyde-reactive substrate at a temperature of about 25° C., about 50° C., about 75° C., or about 100° C. In some cases, heating the aldehyde-reactive substrate is for a time period of about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some cases, reductive-amine condensation comprises heating the aldehyde-reactive substrate at a temperature of about 75° C. for 30 minutes.

In some cases, covalently conjugating the tethered oligonucleotides to the one or more patterns exposing the aldehyde-reactive substrate comprises conducting a reductive-amination step. In some cases, the method comprises conducting a reductive-amination step comprising incubating the aldehyde-reactive substrate in an aqueous solution (e.g. an aqueous solution that does not degrade the tethered oligonucleotides, the cell-resistive layer, and/or cell viability and fitness). In some cases, the method comprises conducting a reductive-amination step comprises incubating the aldehyde-reactive substrate in sodium triacetoxyborohydride. In some cases, the method comprises conducting a reductive-amination step comprises incubating the aldehyde-reactive substrate in sodium cyanoborohydride. In some cases, the method comprises conducting a reductive-amination step comprises incubating the aldehyde-reactive substrate in sodium borohydride. In some cases, the method comprises conducting a reductive-amination step comprises incubating the aldehyde-reactive substrate in sodium borohydride in 1×PBS. In some cases, the sodium borohydride is 0.25% sodium borohydride. In some cases, the aldehyde-reactive substrate is incubated with sodium borohydride for a time period of about 1 minute, 5 minutes, 15 minutes, 30 minutes, 40 minutes, 50 minutes, or 60 minutes at room temperature.

In some cases, following the reductive amination step, the method comprises rinsing the aldehyde-reactive substrate with deionized water. In some cases, the method further comprises removing the cell-resistive layer from the aldehyde-reactive substrate. In some cases, removing the cell-resistive layer comprises rinsing the aldehyde-reactive substrate with acetone. In some cases, removing the cell-resistive layer comprises rinsing the aldehyde-reactive substrate with deionized water. In some cases, removing the cell-resistive layer comprises rinsing the aldehyde-reactive substrate with acetone, followed by deionized water. In some cases, removing the cell-resistive layer comprises rinsing the aldehyde-reactive substrate, and drying the aldehyde-reactive substrate with dry nitrogen gas.

In some cases, removal of the cell-resistive layer results in one or more tethered DNA patterns comprising one or more tethered oligonucleotides attached to the aldehyde-reactive substrate. In some cases, the one or more patterns comprises one or more array of patterns. Non-limiting examples of one or more patterns, one or more array or patterns, and/or microislands is shown in FIG. 26.

The tethered and the untethered oligonucleotides have a length of from about 15 nucleotides (nt) to about 50 nt; e.g., the tethered and the untethered oligonucleotides have a length of from about 15 nt to about 20 nt (e.g., 15 nt, 16 nt, 17 nt, 18 nt, 19 nt, or 20 nt), from about 20 nt to about 25 nt (e.g., 20 nt, 21 nt, 22 nt, 23 nt, 24 nt, or 25 nt), from about 25 nt to about 30 nt (e.g., 25 nt, 26 nt, 27 nt, 28 nt, 29 nt, or 30 nt), from about 30 nt to about 35 nt, from about 35 nt to about 40 nt, from about 40 nt to about 45 nt, or from about 45 nt to about 50 nt.

As noted above, each of the plurality of tethered oligonucleotides comprises a nucleotide sequence that, when hybridized to a complementary nucleotide sequence present in an untethered oligonucleotide, generates an enzyme cleavage site. Such a nucleotide sequence is referred to herein as a “hybridization nucleotide sequence.” The length of the hybridization nucleotide sequence can be from about 6 nucleotides (nt) to about 30 nt; e.g., the length of the hybridization nucleotide sequence can be from about 6 nt to about 10 nt (e.g., 6 nt, 7 nt, 8 nt, 9 nt, or 10 nt), from about 10 nt to about 15 nt (e.g., 10 nt, 11 nt, 12 nt, 13 nt, 14 nt, or 15 nt), from about 15 nt to about 20 nt (e.g., 15 nt, 16 nt, 17 nt, 18 nt, 19 nt, or 20 nt), from about 20 nt to about 25 nt, or from about 25 nt to about 30 nt. Non-limiting examples of nucleotide sequences of the tethered oligonucleotides, include, but are not limited to: A-BamHI-NH₂: ACTGACTGACTGACTGACTGCCATAAGGGATCCCTAAGCA (SEQ ID NO:11); F—NH₂: AGAAGAAGAACGAAGAAGAA (SEQ ID NO:12); G-NH₂: AGCCAGAGAGAGAGAGAGAG (SEQ ID NO:13); G-EcoRI-NH₂ AGCCAGAGAGAGAGAGAGAGCTAAGC AGAATTCCCATAAG (SEQ ID NO:14); B-Cas9-PAM-D′-NH₂, and TCATACGACTCACTCGTCACCTCCAATGACTAGGGTGGGTAACGATCCAG (SEQ ID NO:15).

In some cases, the one or more tethered oligonucleotides are covalently attached to the exposed aldehyde-reactive substrate within the one or more patterns, thereby forming a tethered DNA patterned substrate. The tethered DNA pattern comprises a diameter ranging from 50 nm to 50 mm. In some cases, the one or more tethered DNA patterns comprises a diameter ranging from 50 nm to 100 nm. In some cases, the one or more Tethered DNA patterns comprises a diameter ranging from 50 nm to 100 μm. In some cases, the one or more tethered DNA patterns comprises a diameter ranging from 2 to 5 μm, 5 to 10 μm, 10 to 15 μm, 15 to 20 μm, 20 to 25 μm, 25 to 30 μm, 30 to 35 μm, 35 to 40 μm, 40 to 45 μm, or 45 to 50 μm. In some cases, the one or more tethered DNA patterns comprises a diameter ranging from 50 to 100 μm, 100 to 150 μm, 150 to 200 μm, 200 to 250 μm, 250 to 300 μm, 300 to 350 μm, 350 to 400 μm, 400 to 450 μm, or 450 to 500 μm. In some cases, the one or more tethered DNA patterns comprises a diameter ranging from 1 mm to 5 mm, 5 mm to 10 mm, 10 mm to 15 mm, 15 mm to 20 mm, 20 mm to 25 mm, or 25 mm to 30 mm.

In some cases, the one or more tethered DNA patterns is a cylindrical shape, a circular shape, a square shape, a spherical shape, a cylindrical shape, a rectangular shape, or a combination thereof. In some cases, the one or more tethered DNA patterns comprises one or more micro-islands. In some cases, the one or more tethered DNA patterns comprises one or more tethered DNA patterns selected from the tethered DNA patterns as shown in FIG. 26. The one or more tethered DNA patterns is not limited to the shapes and/or sizes as described herein and can be any shape and/or size as required per conditions specific to its intended use. In some cases, the one or more DNA patterns is a cylindrical shaped DNA pattern, a circular shaped DNA pattern, a square shaped DNA pattern, a spherical shaped DNA pattern, a cylindrical shaped DNA pattern, or a rectangular shaped DNA pattern. In some cases, the one or more DNA patterns is a shaped pattern that mimics a biological structure. In some cases, the one or more patterns of the cell-resistive layer exposing the aldehyde-reactive substrate is shaped as any type of biological tissue. Non-limiting examples of shapes of patterns that mimic a biological structure include, but are not limited to, intestinal folds and/or liver lobules. The one or more DNA patterns is not limited to the shapes and/or sizes as described herein and can be any shape and/or size as required per conditions specific to its intended use.

In some cases, the DNA pattern comprises two or more array of tethered DNA patterns, three or more array of tethered DNA patterns, four or more array of tethered DNA patterns, five or more array of tethered DNA patterns, six or more array of tethered DNA patterns, seven or more array of tethered DNA patterns, eight or more array of tethered DNA patterns, nine or more array of tethered DNA patterns, or ten or more array of tethered DNA patterns separated from one another. In some cases, an array of tethered DNA patterns comprises 2 or more, 5 or more, 10 or more, 20 or more, 30 or more, 40 or more, 50 or more, 60 or more, 70 or more, 80 or more, 90 or more, 100 or more, 120 or more, 130 or more 140 or more, 150 or more, 200 or more, 250 or more 300 or more, 350 or more, 400 or more 450 or more, or 500 or more tethered DNA patterns. In some cases, the grid separates a first array of tethered DNA patterns from a second array of tethered DNA patterns. In some cases, the aldehyde-reactive substrate comprises a 2×2 array of tethered DNA patterns, a 3×3 array of tethered DNA patterns, a 4×4 array of tethered DNA patterns, a 5×5 array of tethered DNA patterns, a 6×6 array of tethered DNA patterns, a 7×7 array of DNA patterns, an 8×8 array of tethered DNA patterns, a 9×9 array of tethered DNA patterns, or a 10×10 array of tethered DNA patterns. In some cases, the aldehyde-reactive substrate comprises a 15×15 array of tethered DNA patterns, a 20×20 array of tethered DNA patterns, a 25×25, a 50×50 array of tethered DNA patterns, a 100×100 array of tethered DNA patterns, a 150×150 array of tethered DNA patterns, or more than 150×150 array of tethered DNA patterns. In some cases, the grid creates one or more micro-islands separated from one another, each comprising an array of tethered DNA patterns. In some cases, the one or more microislands is a cylindrical shape, a circular shape, a square shape, a spherical shape, a cylindrical shape, a rectangular shape, or a combination thereof. In some cases, the one or more microislands comprises a length ranging from 1 μm to 500 μm and a width ranging from 1 μm to 500 μm. In some cases, the one or more microislands comprises a length of 141 μm and a width of 141 μm. In some cases, the one or more microislands comprises a length ranging from 1 mm to 50 mm and a width ranging from 1 mm to 50 mm. In some cases, the one or more microislands comprises one or more microislands selected from the microislands as shown in FIG. 26. The one or more microislands comprising one or more tethered DNA patterns is not limited to the shapes and/or sizes as described herein and can be any shape and/or size as required per conditions specific to its intended use.

In some cases, the method further comprises fabricating a grid, following DNA patterning, to form one or more microislands. In some cases, microislands separate single-cell patterns from each other during culture. In some cases, fabricating a grid comprises applying a cell-resistive layer (e.g. photoresist) over the aldehyde-reactive substrate and heating the cell-resistive layer. In some cases, fabricating the grid further comprises applying a mask comprising one or more patterns to the cell-resistive layer, wherein the one or more patterns of the mask does not expose the one or more DNA pattern. For example, the one or more patterns of the mask are configured to pattern areas of the aldehyde-reactive substrate that are not conjugated to a tethered oligonucleotide. In some cases, the method of fabricating the grid further comprises exposing the aldehyde-reactive substrate, the cell-resistive layer, and the mask to UV light. The UV light solubilizes the cell-resistive layer where tethered DNA patterns are not present, and the remaining cell-resistive layer that is not solubilized protects the tethered DNA patterns. The method of fabricating the grid further comprises dropcasting a polymeric material (e.g. polyacrylamide) across the aldehyde-reactive substrate, reacting with the exposed aldehyde groups within unpatterned cell-resistive areas. In some cases, the polymer is a polyacrylamide. In some cases, the method of fabricating the grid comprises dropcasting a 10% polyacrylamide solution in 10 mM 4-(2-hydroxyethyl)-1-piperazineethanesulfonic acid) (HEPES, pH=7). In some cases, prior to dropbasting the polyacrylamide solution, the method comprises degassing the polyacrylamide solution in a desiccator for a period of time (e.g., 10 minutes) to remove dissolved oxygen. In some cases, the method further comprises activating the polyacrylamide solution with 1.5% tetramethylethylenediamine (Bio-Rad) and 0.225% ammonium persulfate (Bio-Rad), the polyacrylamide mixture was dropcast immediately over the photoresist features, and a Gel Slick (Lonza)-treated glass coverslip is used to spread out the polyacrylamide solution over the entire DNA-patterned substrate. In some cases, following 1 hour of polymerization, the method comprises removing a coverslip from the aldehyde-reactive substrate, and rinsing the aldehyde-reactive substrate with deionized water to remove unreacted polyacrylamide. In some cases, the method further comprises removing a cell-resistive layer defining the polyacrylamide patterns and protecting the tethered DNA pattern by dissolving in acetone. In some cases, the method further comprises rinsing the aldehyde-reactive substrate with Deionized water, and drying the aldehyde-reactive substrate with dry nitrogen gas.

In some cases, the method of the present disclosure comprises flowing one or more untethered oligonucleotides of the present disclosure over the one or more DNA patterns with a flow cell, as described herein. In some cases, the solid support comprises one or more flow cells. In some cases, the one or more flow cells comprises a flow cell chamber. In some cases, the flow cell chamber is configured to flow the untethered oligonucleotides of the present disclosure across the surface of the flow cell chamber. In some cases, the flow cell comprises an inlet. In some cases, the flow cell comprises an outlet.

In some cases, the method comprises positioning one or more flow cells over the one or more patterns exposing the aldehyde-reactive substrate. In some cases, the one or more flow cells are positioned over the one or more patterns exposing the aldehyde-reactive substrate. In some cases, the method comprises positioning the one or more flow cells over a microisland surrounding the one or more patterns, or an more array of patterns, exposing the aldehyde-reactive substrate. In some cases, the method comprises affixing the one or more flow cells to the solid support substrate such that each flow cell is positioned over the one or more microislands comprising one or more patterns, or an array of patterns. In some cases, the one or more patterns, or an array of patterns is contained within the flow cell chamber. In some cases, the one or more flow cells are made from a polymeric material. Non-limiting examples of polymeric materials include, but are not limited to, plastics (for example, polytetrafluoroethylene, polypropylene, polystyrene, polycarbonate, and blends thereof, and the like), PDMS, polysaccharides such as agarose and dextran, polyacrylamides, polystyrenes, polyvinyl alcohols, copolymers of hydroxyethyl methacrylate and methyl methacrylate, and the like.

In some cases, the one or more flow cells is configured to flow one or more untethered oligonucleotides across the solid support comprising the one or more tethered oligonucleotides. For example, the one or more flow cells concentrates the cells to the surface of the solid support and increases the probability of hybridization between the untethered oligonucleotides and the tethered oligonucleotides.

In some cases, the method further comprises flowing, onto the one or more patterns: i) one or more cells; ii) one or more effector molecules; or iii) a combination of i) and ii), wherein the one or more cells and the one or more effector molecules is bound to one or more untethered oligonucleotides comprising the complementary nucleotide sequence to the one or more tethered oligonucleotide sequences on the one or more patterns exposing the aldehyde-reactive substrate.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-54 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising:
a) a solid support;
b) a plurality of tethered oligonucleotides, wherein the tethered oligonucleotides are attached to the solid support via the 5′ termini of the oligonucleotides in a patterned array, wherein each of the plurality of tethered oligonucleotides comprises a nucleotide sequence that, when hybridized to a complementary nucleotide sequence present in an untethered oligonucleotide, generates an enzyme cleavage site; and
c) a plurality of untethered oligonucleotides that are hybridized to the plurality of tethered oligonucleotides in the patterned array, wherein the untethered oligonucleotides each comprise:
   i) the nucleotide sequence that generates an enzyme cleavage site, wherein the enzyme cleavage site is a restriction enzyme cleavage site or a site that is cleavable by a CRISPR/Cas effector polypeptide;
   ii) a cell, or an effector molecule that affects an activity and/or a phenotype of a cell, wherein the cell or the effector molecule is attached to the untethered oligonucleotide at the 5′ end of the untethered oligonucleotides; and
   iii) a fluorophore.

Aspect 2. The composition of aspect 1, wherein the effector molecule is a polypeptide.

Aspect 3. The composition of aspect 1, wherein the effector molecule comprises a lipid.

Aspect 4. The composition of aspect 1, wherein the effector molecule comprises an oligosaccharide.

Aspect 5. The composition of any one of aspects 1-4, wherein the enzyme cleavage site is:
a) a restriction enzyme cleavage site; or
b) a site that is cleavable by a CRISPR/Cas effector polypeptide when the CRISPR/Cas effector polypeptide is complexed with a guide RNA.

Aspect 6. The composition of any one of aspects 1-5, wherein the composition comprises at least a first, a second, and a third plurality of hybridized, untethered oligonucleotides that are bound to the tethered oligonucleotides in the patterned array, wherein:
a) the first plurality of hybridized, untethered oligonucleotides comprises, bound to the 5′ end of the oligonucleotides, a first effector molecule, wherein the first plurality of hybridized, untethered oligonucleotides generates a first enzyme cleavage site; and
b) the second plurality of hybridized, untethered oligonucleotides comprises, bound to the 5′ end of the oligonucleotides, a second effector molecule, wherein the second plurality of hybridized, untethered oligonucleotides generates a second enzyme cleavage site; and
c) the third plurality of hybridized, untethered oligonucleotides comprises, bound to the 5′ end of the oligonucleotides, a target cell.

Aspect 7. The composition of aspect 6, further comprising a fourth plurality of hybridized, untethered oligonucleotides, wherein the fourth plurality of hybridized, untethered oligonucleotides bound to the 5′ end of the oligonucleotides, a third effector molecule, wherein the fourth plurality of hybridized, untethered oligonucleotides generates a third enzyme cleavage site.

Aspect 8. The composition of any one of aspects 1-7, wherein the cell is a stem cell or a progenitor cell.

Aspect 9. The composition of any one of aspects 1-8, wherein the effector molecule is a growth factor, a hormone, an adhesion protein, a tumor-associated antigen, an integrin, a chemokine, a juxtacrine, an. antibody, an extracellular matrix polypeptide, a co-stimulatory polypeptide, a T-cell receptor, a morphogen, a delta family protein, a Notch family protein, a Wnt polypeptide, or an Eph polypeptide.

Aspect 10. A method of temporally modulating an activity and/or phenotype of a cell, the method comprising:
a) at a first time, contacting the composition of any one of aspects 2-9 with a first enzyme that cleaves the first enzyme cleavage site, wherein said contacting results in removal of the first effector molecule from the target cell; and
b) determining the effect of the removal of the first effector molecule on an activity and/or phenotype of the cell.

Aspect 11. The method of aspect 10, comprising:
c) at a second time, contacting the composition of any one of aspects 2-6 with a second enzyme that cleaves the second enzyme cleavage site, wherein said contacting results in removal of the second effector molecule from the target cell; and
d) determining the effect of the removal of the second effector molecule on an activity and/or phenotype of the cell.

Aspect 12. A solid support comprising:
a) one or more patterns exposing an aldehyde-reactive substrate;
b) one or more tethered oligonucleotides covalently attached to the exposed aldehyde-reactive substrate within the one or more patterns via an amine-modified terminus at the 5′ end of the one or more tethered oligonucleotides,
wherein the one or more tethered oligonucleotides comprises a nucleotide sequence that, when hybridized to a complementary nucleotide sequence present in an untethered oligonucleotide, generates an enzyme cleavage site.

Aspect 13. The solid support of aspect 12, wherein the enzyme cleavage site is:
a) a restriction enzyme cleavage site; or
b) a site that is cleavable by a CRISPR/Cas effector polypeptide when the CRISPR/Cas effector polypeptide is complexed with a guide RNA.

Aspect 14. The solid support of aspect 12 or aspect 13, wherein the one or more tethered oligonucleotides comprises a nucleotide sequence that hybridizes to a complementary nucleotide sequence present in an untethered oligonucleotide, generates an enzyme cleavage site, and wherein the untethered oligonucleotide comprises a target cell bound to the 5′ end of the untethered oligonucleotide.

Aspect 15. The solid support of aspect 12 or aspect 13, wherein the one or more tethered oligonucleotides comprises a nucleotide sequence that hybridizes to a complementary nucleotide sequence present in an untethered oligonucleotide, generates an enzyme cleavage site, and wherein the untethered oligonucleotide comprises an effector molecule bound to the 5′ end of the untethered oligonucleotide.

Aspect 16. The solid support of any one of aspects 12-15, wherein the aldehyde-reactive substrate further comprises a grid that surrounds the one or more patterns.

Aspect 17. The solid support of any one of aspects 9-16, wherein the aldehyde-reactive substrate further comprises one or more alignment markers.

Aspect 18. The solid support of any one of aspects 9-17, wherein the one or more tethered oligonucleotides has a length of from 20 nucleotides to 50 nucleotides.

Aspect 19. The solid support of any one of aspects 9-18, wherein the one or more patterns exposing the aldehyde-reactive substrate comprises a diameter ranging from 50 nm to 50 mm.

Aspect 20. The solid support of any one of aspects 9-19, wherein the one or more patterns exposing the aldehyde-reactive substrate comprises a diameter ranging from 2-5 μm, 5-10 μm, 10-15 μm, 15-20 μm, 20-25 μm, or 25-30 μm.

Aspect 21. The solid support of any one of aspects 9-20, wherein the one or more patterns comprises one or more micro-islands.

Aspect 22. The solid support of any one of aspects 9-21, wherein the grid is a polyacrylamide grid.

Aspect 23. The solid support of any one of aspects 9-22, further comprising one or more flow cells.

Aspect 24. The solid support of aspect 23, wherein the one or more flow cells is positioned over the plurality of tethered oligonucleotides.

Aspect 25. A method of making a DNA patterned surface, the method comprising:
a) functionalizing a surface of a solid support with aldehyde groups to form an aldehyde-reactive substrate;
b) applying a cell-resistive layer onto the aldehyde-reactive substrate;
c) heating the cell-resistive layer;
d) applying a mask comprising one or more patterns to the cell-resistive layer;
e) exposing the aldehyde-reactive substrate, the cell-resistive layer, and the mask to create one or more patterns exposing the aldehyde-reactive substrate;
f) flowing one or more tethered oligonucleotides over the one or more patterns exposing the aldehyde-reactive substrate, wherein the one or more tethered oligonucleotides comprises a nucleotide sequence that, when hybridized to a complementary nucleotide sequence present in an untethered oligonucleotide, generates an enzyme cleavage site;
g) conjugating the 5′ amine-modified end of the one or more tethered oligonucleotides to the exposed aldehyde-reactive substrate within the one or more patterns; and
h) removing the cell-resistive layer.

Aspect 26. The method of aspect 25, wherein the method further comprises repeating steps b)-h) to create layers of the one or more patterns.

Aspect 27. The method of aspect 25, wherein the method further comprises, before step f), diluting the tethered oligonucleotides in a buffer.

Aspect 28. The method of aspect 27, wherein the method further comprises, before step g), incubating the one or more tethered oligonucleotides for about 5 minutes.

Aspect 29. The method of aspect 27, wherein the buffer is a sodium phosphate buffer.

Aspect 30. The method of aspect 29, wherein the concentration of the sodium phosphate buffer is about 50 mM.

Aspect 31. The method of aspect 28, wherein the method further comprises heating the solid support.

Aspect 32. The method of aspect 27, wherein the solid support is heated at 75° C. for about 60 minutes.

Aspect 33. The method of aspect 25, wherein said conjugating comprises performing amine-condensation comprising adding sodium dodecyl sulfate to the solid support.

Aspect 34. The method of aspect 33, wherein amine-condensation further comprises incubating the solid support at a temperature of from 90° C. to 100° C.

Aspect 35. The method of aspect 34, wherein the solid support is incubated for a period of time of from about 5 minutes to about 60 minutes.

Aspect 36. The method of aspect 34, wherein the incubation temperature is 75° C.

Aspect 37. The method of aspect 35, wherein the solid support is incubated for 30 minutes.

Aspect 38. The method of aspect 35, wherein the method further comprises rinsing the solid support with water.

Aspect 39. The method of aspect 25, wherein said conjugating comprises performing reductive-amination comprising adding sodium borohydride to the solid support.

Aspect 40. The method of aspect 39, wherein the method further comprises incubating the solid support for a period of time of from about 1 minute to about 30 minutes.

Aspect 41. The method of aspect 25, wherein removing the cell-resistive layer comprises:
i. rinsing the solid support; and r
ii. drying the solid support.

Aspect 42. The method of aspect 41, wherein said rinsing comprises rinsing the solid support with acetone.

Aspect 43. The method of aspect 41, wherein said drying comprises drying the solid support with nitrogen gas.

Aspect 44. The method of aspect 25, wherein the one or more tethered oligonucleotides are conjugated orthogonally relative to the aldehyde-reactive substrate.

Aspect 45. The method of aspect 25, wherein the target nucleotide sequence, when hybridized to a complementary nucleotide sequence present in an untethered oligonucleotide, generates a restriction enzyme cleavage site or a site that is cleavable by a CRISPR/Cas effector polypeptide complexed with a guide RNA.

Aspect 46. The method of aspect 25, further comprising flowing, onto the one or more patterns:
i) one or more cells;
ii) one or more effector molecules; or
iii) a combination of i) and ii),
wherein the one or more cells and the one or more effector molecules is bound to one or more untethered oligonucleotides comprising the complementary nucleotide sequence to the one or more tethered oligonucleotide sequences on the one or more patterns exposing the aldehyde-reactive substrate.

Aspect 47. The method of aspect 25, wherein the method comprises two or more tethered oligonucleotide sequences.

Aspect 48. The method of any one of aspects 25-47, wherein the cell-resistive layer is a photoresist layer.

Aspect 49. The method of any one of aspects 25-48, wherein the mask is a photomask.

Aspect 50. The method of any one of aspects 25-47, wherein the mask is an optical mask.

Aspect 51. The method any one of aspects 25-49, wherein the radiation is a beam of light.

Aspect 52. The method of any one of aspects 25-47, wherein the radiation is a beam of electrons.

Aspect 53. The method of any one of aspects 25-47, wherein the radiation is a beam of ions.

Aspect 54. A solid support comprising:
a) one or more patterns exposing an aldehyde-reactive substrate;
b) one or more tethered oligonucleotides, covalently attached to the one or more patterns via an amine-modified termini at the 5′ end of the plurality of tethered oligonucleotides, wherein the one or more of tethered oligonucleotides comprises a nucleotide sequence that, when hybridized to a complementary nucleotide sequence present in an untethered oligonucleotide, generates a restriction enzyme cleavage site or a site that is cleavable by a CRISPR/Cas effector polypeptide;
c) one or more untethered oligonucleotides.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

A Multiplexed, Lithographic DNA Approach to Recapitulate Complex Signaling Environments with Controlled Spatial Presentations

### Results

Fabricating Instructive, Multicomponent Surface DNA Patterns with Spatial and Hierarchical Complexity using Photolithography

While DNA-instructed assembly provides a simple and robust solution to coordinate heterogeneous signaling components by capitalizing on the specificity and strong, rapid binding kinetics of Watson-Crick base pairing, the challenge of engineering a parallel approach to fabricate multicomponent DNA-instructive surfaces was tackled. A microcantilever-based printing technology was employed to spot oligonucleotides as cell-sized circular features that then capture and assemble heterogeneous cell communities at the single-cell level. The reproducibility, throughput, and multiplexing capabilities of this method, however, were severely limited. DNA patterns were restricted to those comprised of spotted DNA features that were highly dependent on the humidity of the chamber enclosing the cantilever-based system and on human trial-and-error in identifying the appropriate printing conditions (i.e. printing speed, cantilever contact time, etc.). Furthermore, while the system was programmable to be semi-automated, the time required for complex printing of even a single oligonucleotide could be hours due to the method's inherently serial nature, effectively precluding its application to printing and registering multiple oligonucleotides.

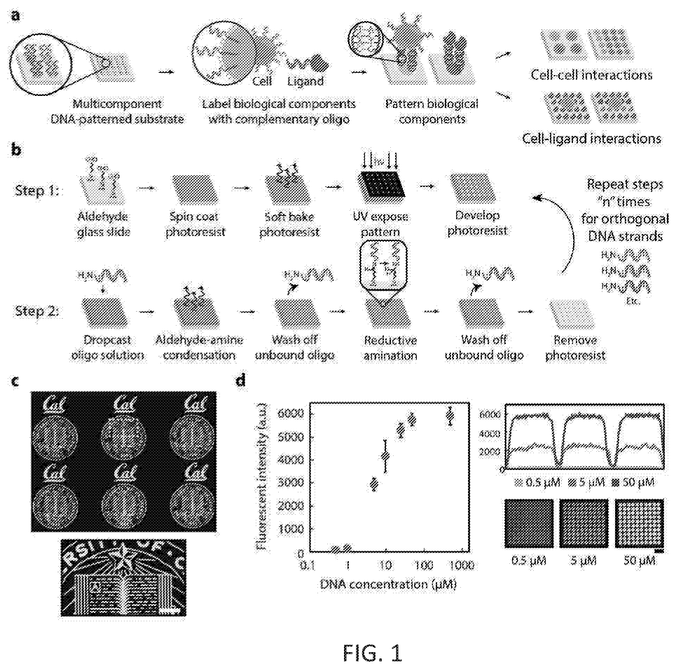

Here, a major advance of the underlying surface DNA-patterning concept is presented not only by expanding the patterning capabilities of DNA-based assembly to encompass both solid-phase ligands and cells but also by developing a high-throughput, parallel strategy that has increased multiplexing capabilities, flexibility to pattern any geometry, high spatial resolution, and ease in registering multiple DNA layers. Specifically, a strategy that uses patterned photoresist as a physical template was engineered—one that can be iteratively removed and re-patterned to conjugate numerous, orthogonal oligonucleotide strands. As illustrated in FIG. 1 b, there are two key steps for achieving this multicomponent, DNA-patterned platform. The first involves traditional photolithography, where patterned photoresist serves to 1) expose selective areas of surface aldehyde groups for DNA conjugation, 2) act as a physical barrier to prevent conjugation to unexposed aldehyde groups, and 3) preserve protected, unconjugated aldehyde functionality for subsequent, multilayered DNA patterning steps. The second step covalently immobilizes 20 base-pair oligonucleotides to the glass substrate by reacting the primary amine group at the 5′ end of the DNA with the surface-exposed aldehyde groups. See Methods for additional protocol details.

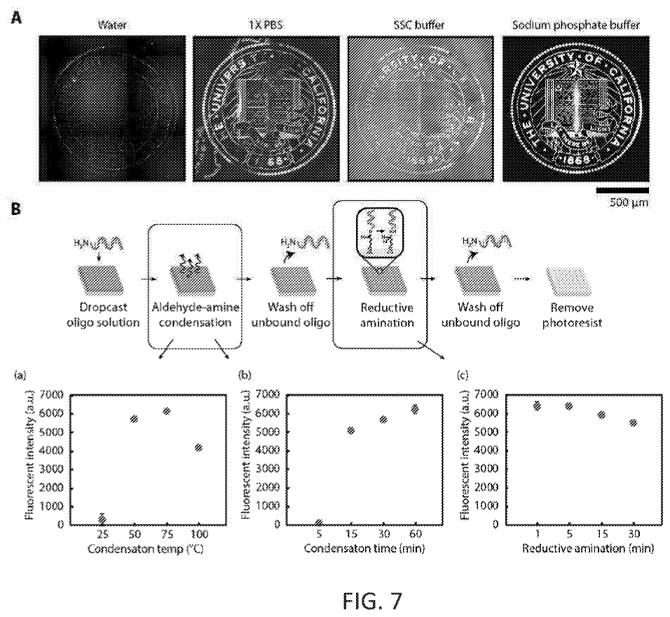

The approach of UV-patterning photoresist offers the distinct advantage of defining, with great control and precision, complex spatial patterns across different length scales (i.e. from microns to millimeters and, in turn, from sub-cellular to bulk population) and over large areas (up to thousands of mm²) within minutes. FIG. 1 c shows the resulting high-resolution surface DNA patterns that can be achieved with this method, visualized by hybridizing a complementary, fluorescent-labeled oligonucleotide. In-depth characterization of DNA-patterning steps revealed that optimization of both the buffer composition and the combination of condensation time and temperature played critical roles in achieving robust surface DNA patterns (FIG. 7). An additional key advantage of the method is the tunable control over DNA concentrations patterned onto the substrate. Such control enables variations in signal concentration, as is found with morphogen gradients during development. By varying the concentration of the oligonucleotide solution dropcast over the photoresist patterns, a >100-fold range of fluorescent intensities was achieved (FIG. 1 d).

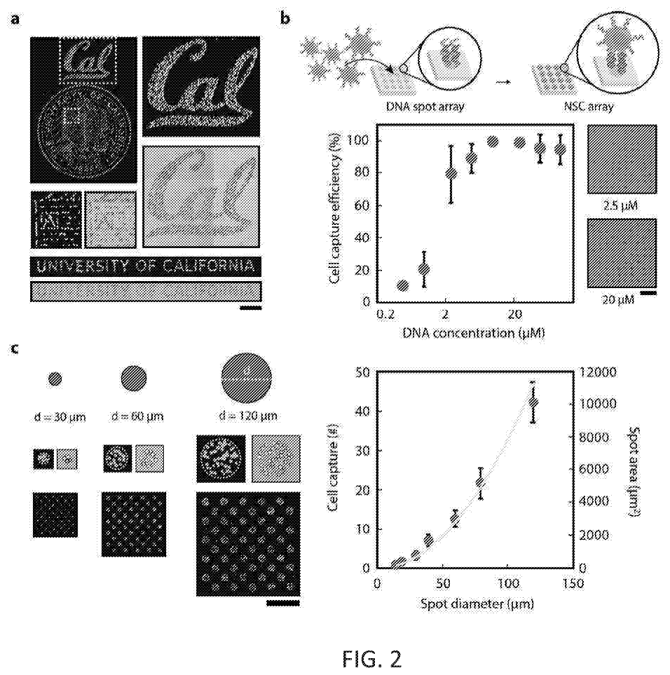

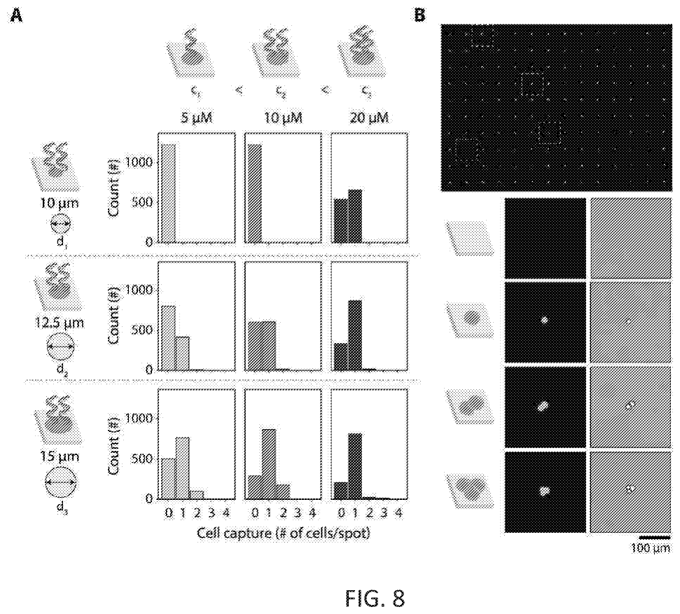

To highlight the utility of this DNA-based engineering approach for modeling spatial heterogeneity in vitro, it was demonstrated that lithographically-defined DNA surfaces are highly functional. As shown in FIG. 2 a, microfabricated DNA patterns were capable of organizing a bulk, oligo-labeled NSC population with high spatial precision, demonstrating the potential to re-create complex cell-based tissue structures, such as the hippocampal dentate gyrus, crypts of intestinal villi, or hepatic lobules. To determine the minimum DNA concentration necessary for cell patterning, cell-capture efficiencies of 20 μm-diameter spot arrays over a concentration range from 0.5-100 μM were tested. The results show that a minimum concentration of 5 μM was necessary to capture at least one oligonucleotide-labeled NSC per spot (FIG. 2 b). Above this concentration, a high average capture rate of >90% was observed. While DNA concentration can influence cell capture, so too can microfabricated pattern feature size. Increasing the diameter of DNA-patterned circle features resulted in a robust and reproducible increase in the number of cells captured per spot (FIG. 2 c). Moreover, when the diameter was commensurate with the size of NSCs (˜15 μm), single-cell capture was achieved (FIG. 8). As is demonstrated herein, this capability enables high-throughput clonal analysis.

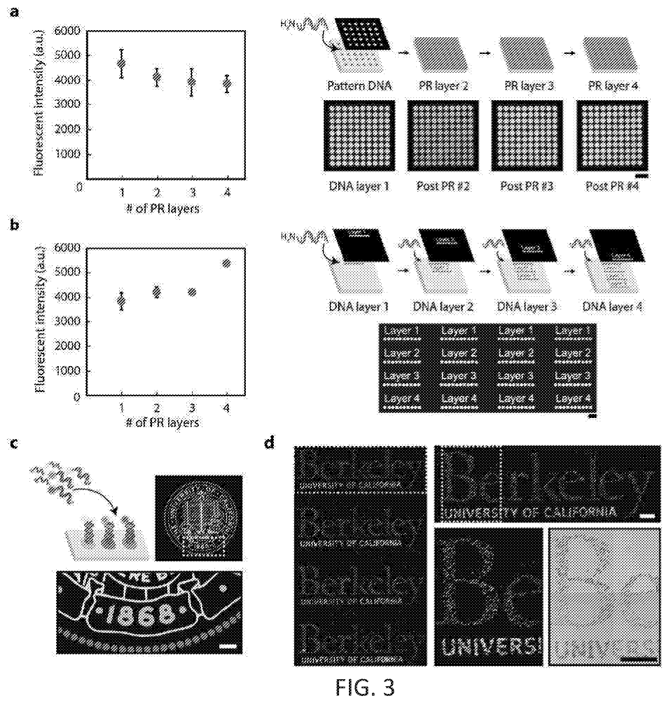

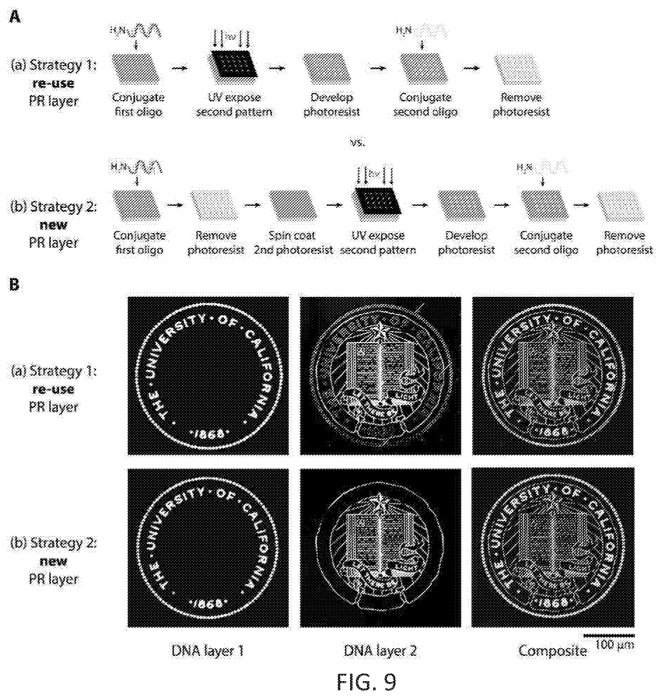

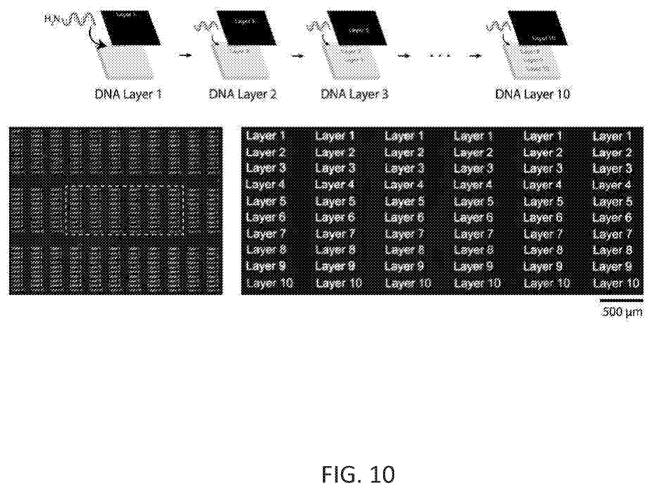

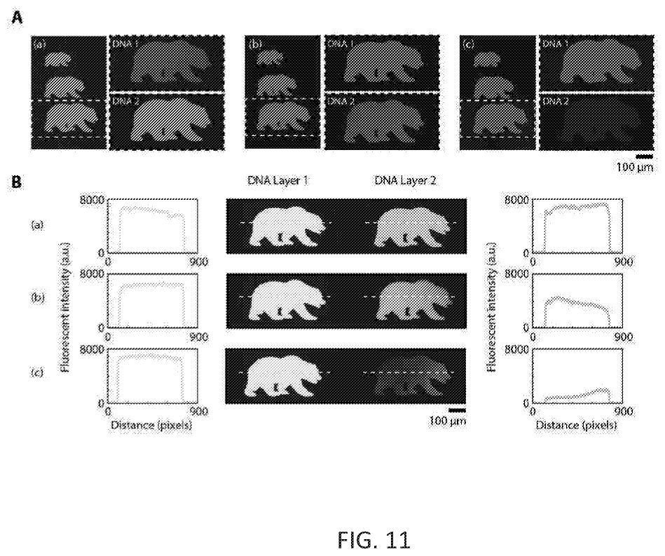

To fabricate multicomponent DNA patterns, it was found that the aforementioned two-step process can be repeated after dissolving the patterned photoresist in acetone and spinning on a new photoresist layer to define a new spatial mask that then guides the conjugation of additional oligonucleotide strands. To validate the robustness and reproducibility of the engineering approach, two key potential pitfalls were examined. First, it was demonstrated that the repeated application and patterning of a new photoresist mask does not adversely affect the first DNA-patterned layer. As FIG. 3A shows, the first DNA pattern retained functionality (i.e. the ability to hybridize) when subjected to iterative removal and application of three additional photoresist layers. In contrast, re-patterning the same photoresist layer for a second DNA pattern resulted in contamination of the first DNA layer (FIG. 9). Thus, the application of new photoresist layers preserves the integrity of the previously-patterned DNA layers (e.g. tethered DNA layers), while also allowing for the selective exposure of additional aldehyde regions for multicomponent conjugation. Second, it was demonstrated that the actual photolithographic steps—particularly, 1) heating, 2) photoresist removal with acetone, and 3) resist development with a highly alkaline solution (pH>10)—do not compromise the aldehyde groups on the glass substrate, as DNA patterns fabricated from subsequent layers retained high-intensity fluorescent values (FIG. 3 b). Extensive characterization established that multilayer patterning can be extended to at least 10 layers without loss of fidelity (FIG. 10) and that tunability of patterned DNA concentrations can also be achieved for multiplexed DNA patterns (FIG. 11). FIG. 3 c and d demonstrates the successful registration of three complex DNA patterns and the robust functionality of multiplexed surface DNA patterns, respectively.

Multicomponent DNA Patterns Instruct the Presentation of Heterogeneous Proteins with High Spatial Control

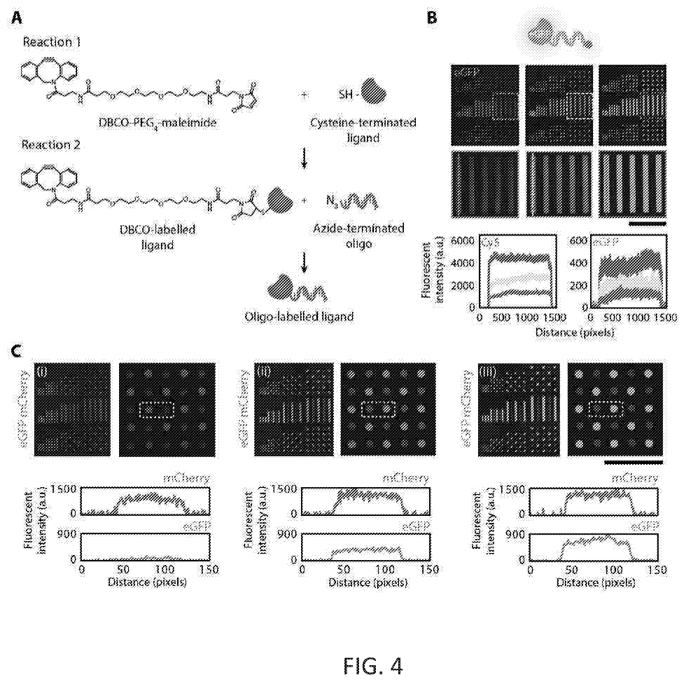

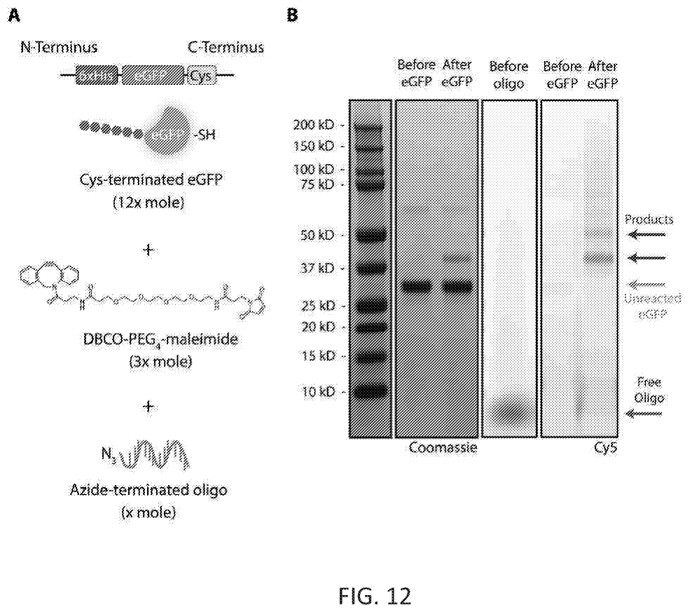

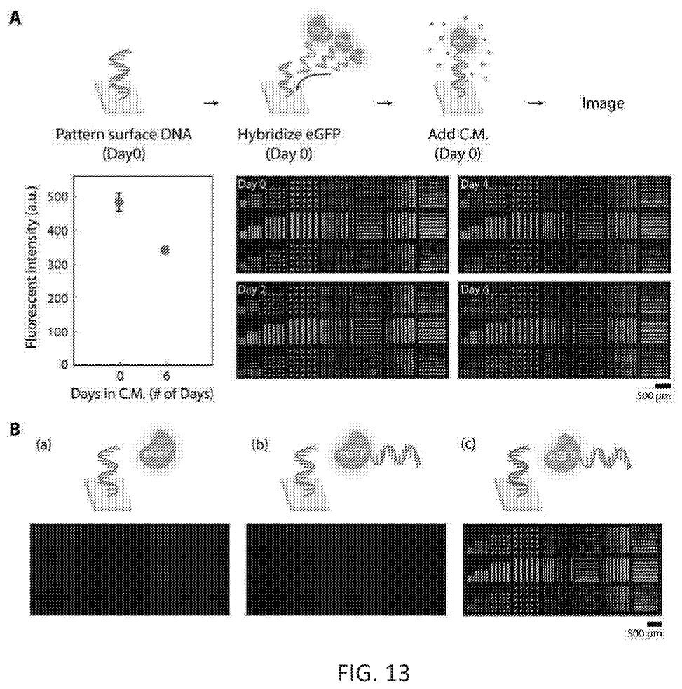

A key step for demonstrating the unique capabilities of the DNA-directed strategy is utilizing the surface DNA patterns to control the spatial organization of solid-phase ligands. Having such control would enable, for instance, emulating the presentation of ECM-sequestered or cell surface-tethered signals. The approach of using DNA as a programmable intermediary capture agent ensures that multiple ligands, each labeled with a different complementary oligonucleotide, can be assembled from a single mixed solution flowed across the DNA-patterned surface. To label ligands of interest with oligonucleotides, the heterobifunctional linker, dibenzocylcooctyene (DBCO)-polyethyleneglycol (PEG4)-maleimide (FIG. 4 a) was utilized. Briefly, ligands were designed to contain a free terminal cysteine to react with the maleimide group on the crosslinker, thereby introducing a DBCO moiety on the ligand that allowed for subsequent click chemistry reaction with an azide-modified oligonucleotide label. As a proof-of-concept, an oligonucleotide was conjugated to recombinant enhanced green fluorescent protein (eGFP) (FIG. 12). Surface DNA patterns directed the spatial organization of eGFP and successfully maintained robust protein patterns over long-term cell culture (FIG. 13 a). In addition, neither eGFP lacking an oligonucleotide label nor eGFP containing a non-complementary label resulted in protein capture (FIG. 13 b), indicating that complementary DNA sequences were necessary to achieve high specificity of eGFP patterns. To visualize patterned ligands and to serve as a relative readout of patterned protein concentration, a Cy5 dye was included at the 3′ end of the oligonucleotide label (FIG. 4 b). Finally, to highlight that spatial control could be extended to multiple solid-phase cues, a second oligo strand was conjugated to mCherry and tunable patterns of mCherry and eGFP were conjugated (FIG. 4 c).

### Applying Multiplexed Surface DNA Patterns to Dissect the Role of Spatial Organization on Competing NSC-Fate Decisions

As a first biological demonstration of the utility of the method, modeling complex signaling scenarios within the adult NSC niche was focused on. Stem cell niches are canonical examples of specialized microenvironments that coordinate the behavior (i.e. quiescence, activation, survival, migration, lineage commitment, etc.) of residing stem cells in response to physiological or pathological directives. Stem cells must decide whether to self-renew, thereby actively contributing to the reserve of stem cells within the niche, or to differentiate into specialized, mature progeny. The complex balance between these two competing fate choices ensures that a stem cell population can maintain homeostasis as well as respond to organismal needs. Here, how adult hippocampal NSCs resolve the competition between opposing fate cues and to what extent spatial organization of a signal offers biophysical context to inform this decision were investigated. The technique's spatial control over both cells and solid-phase cues to recapitulate and modulate NSC interactions with two fate-conflicting niche signals, FGF-2 and ephrin-B2, was leveraged. While FGF-2 operates as both a soluble and ECM-sequestered cue to promote proliferation and stem cell maintenance, ephrin-B2 is a key juxtacrine signal presented by neighboring hippocampal astrocytes that drives neuronal differentiation by signaling through the EphB4 receptor on NSCs. The advantages of the high spatial control of the DNA-based system to model solid-phase competition between FGF-2 and ephrin-B2 were utilized, thereby exposing single-NSC cultures to distinct spatial organizations of the ligands and conducted time-lapse experiments over the course of differentiation to study the dynamics of the single-cell cultures and correlate them with endpoint cell fate.

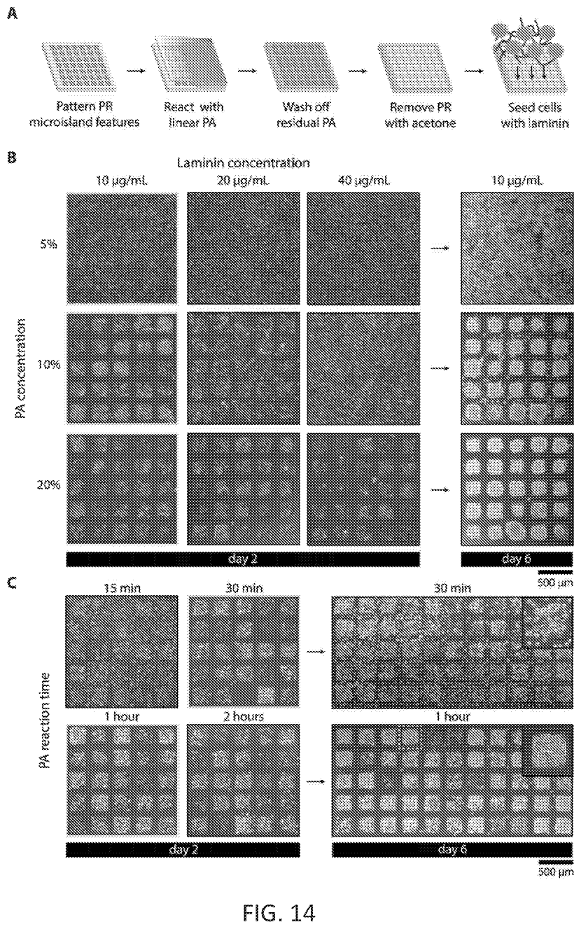

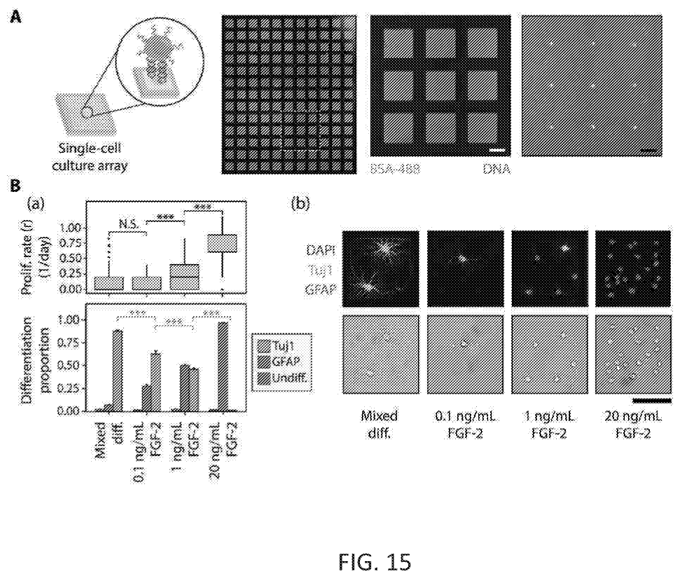

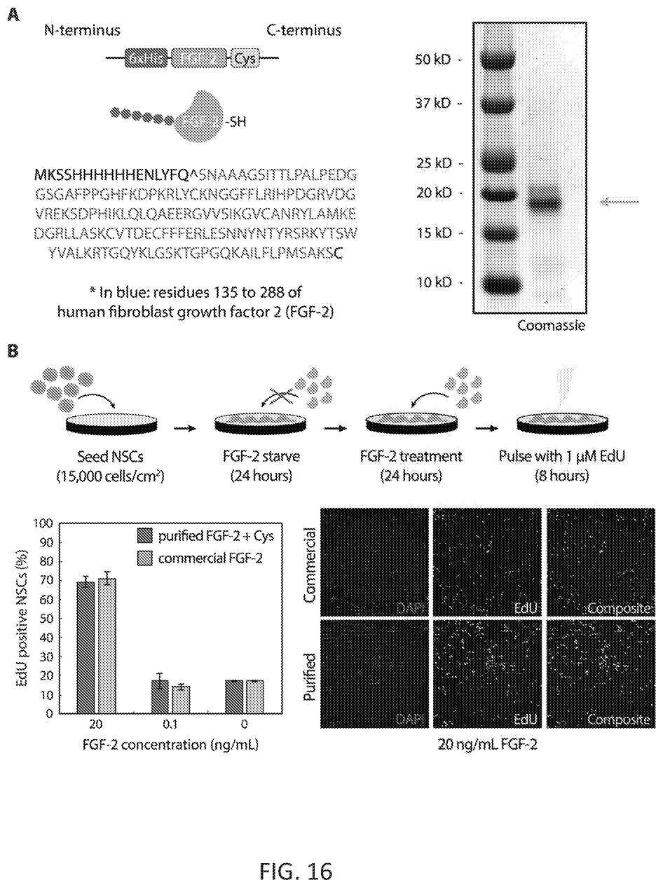

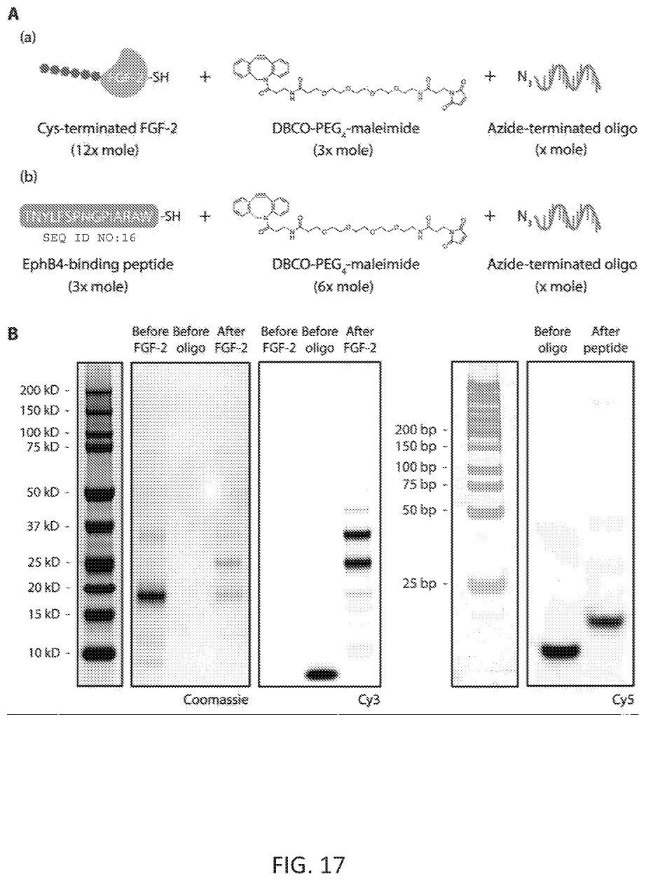

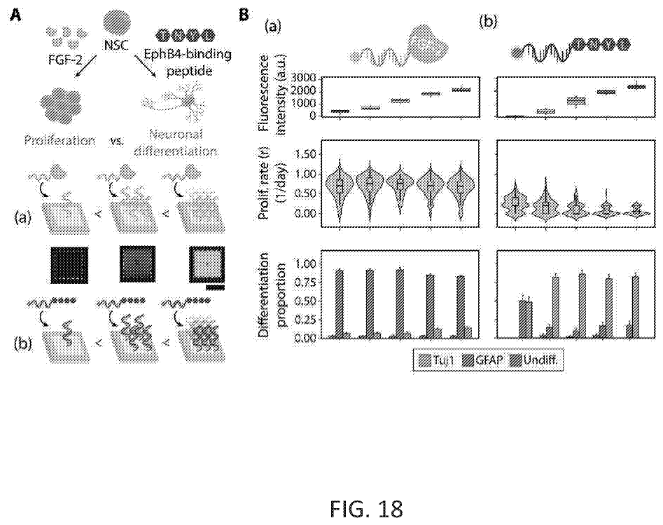

To enable high-throughput clonal analysis in the DNA-based platform, arrays of 15 μm-diameter DNA spots were fabricated to direct the capture of oligonucleotide-labeled single NSCs. Photolithography was then used to pattern a cell-resistive, non-biofouling material, polyacrylamide (PA), to create “microisland” features that isolate single-cell patterns from each other during culture (FIG. 14). The combination of DNA and PA patterns provided the high-throughput power of tracking 1000's of single NSCs over a 5-day differentiation period and subsequently performing clonal analysis by immunostaining to probe for cell fate (FIG. 15). To investigate first the contribution of solid-phase presentation of the individual niche cues, FGF-2 or ephrin-B2, on NSC-fate decisions, both ligands with unique oligonucleotides labeled with a Cy3 and Cy5 fluorescent dye, respectively, were prepared for visualization (FIGS. 16 and 17) and the complementary oligonucleotide was patterned within single NSC microislands to direct the subsequent ligand-oligo conjugate. Because of the challenge of producing recombinant ephrin-B2 in high yield, the full-length protein was replaced with a mimetic peptide, TNYLFSPNGPIARAW (SEQ ID NO:16), that exhibits nanomolar binding affinity to its cognate EphB4 receptor. As expected, FGF-2 and the ephrin-B2 peptide individually promoted opposing cell fates (FIG. 18). Interestingly, immobilized FGF-2 was a potent activator of high proliferation and low differentiation across all patterned ligand concentrations in contrast to the lower proliferation rate and higher differentiation observed with decreasing concentrations of soluble FGF-2 (FIG. 15). The results suggest that the activity of FGF-2 is more potent as a solid-phase than as a soluble cue, consistent with prior work on other immobilized growth factors. For the ephrin-B2 peptide, a minimum concentration threshold was necessary before observing high neuronal differentiation and low proliferation rate. Moreover, with increasing peptide concentrations, heterogeneity of single-cell microislands decreased, converging onto a similar end fate of a single differentiated neuron.

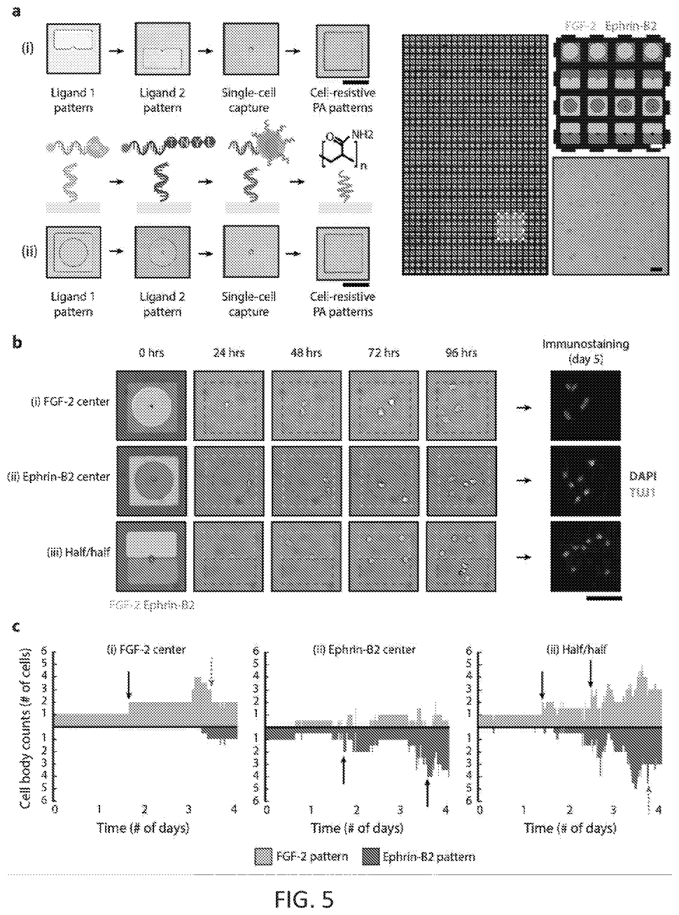

AsFGF-2 and the ephrin-B2-mimetic peptide were observed to drive divergent cell fates in single NSCs, the DNA-based method was employed to assemble and model scenarios in which NSCs are presented with both conflicting cues, thereby emulating the expression of both of these signals by hippocampal astrocytes contacting NSCs. The system's spatial control over both ligands and cells to modulate ligand presentation within single-cell microislands cultures was capitalized upon. Two DNA patterning strategies were implemented in parallel (FIG. 5 a). The first involved constraining single NSCs to the center of either an FGF-2 region or ephrin-B2 peptide region, with the second ligand patterned at the microisland periphery. The second strategy positioned single NSCs at the interface between two, half/half ligand patterns with equal access to both solid-phase signals. Thousands of microislands encompassing all three spatial arrangements were assembled simultaneously, as shown in FIG. 5 a (right).

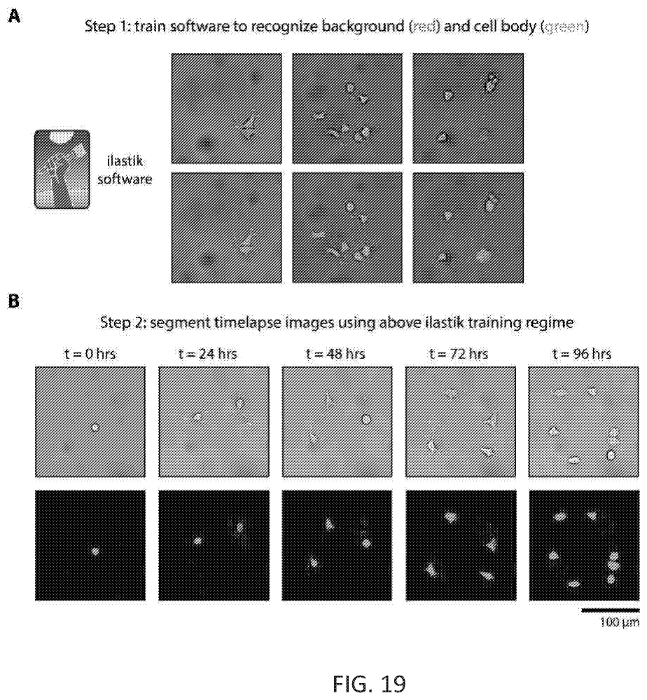

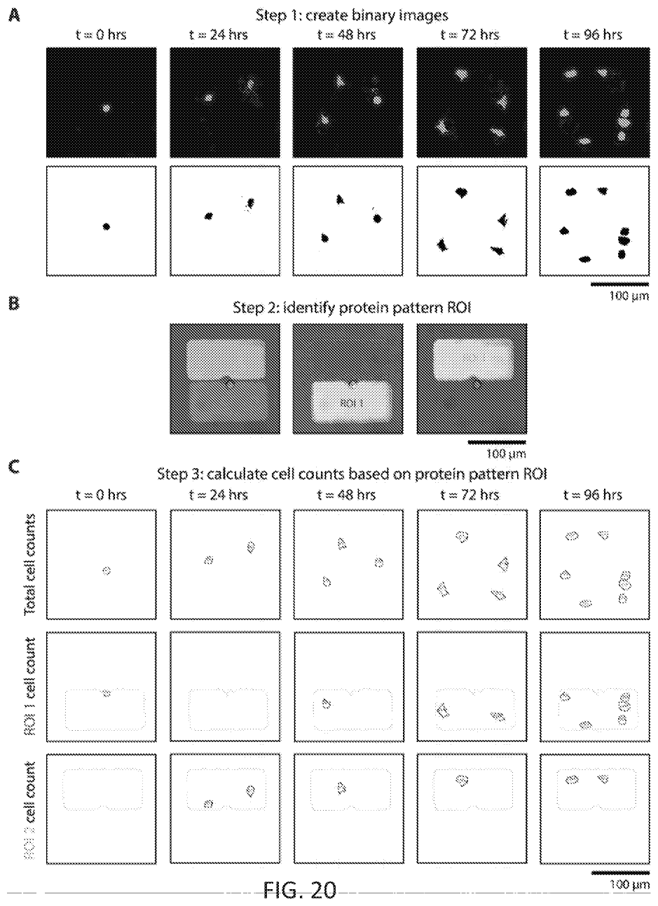

To assess whether variations in ligand spatial organization were sufficient to alter NSCs interactions with the two solid-phase signals and also whether this ultimately translated to differences in end-fate decisions, microislands were imaged over the course of a 4-day time-lapse to track cell body distributions across the ligand-patterned regions. Time-lapse snapshots of (i) FGF-2-center, (ii) ephrin-B2-center, and (iii) half/half microislands are provided in FIG. 5 b (left) along with their corresponding day 5 immunostaining results (right). Custom computational analysis (FIGS. 19 and 20) provided the capability to map out the dynamic cell-ligand interactions of each microisland culture and track how the initial parent NSC and its subsequent progeny distributed themselves over time in response to the organization of these competing niche cues (FIG. 5 c).

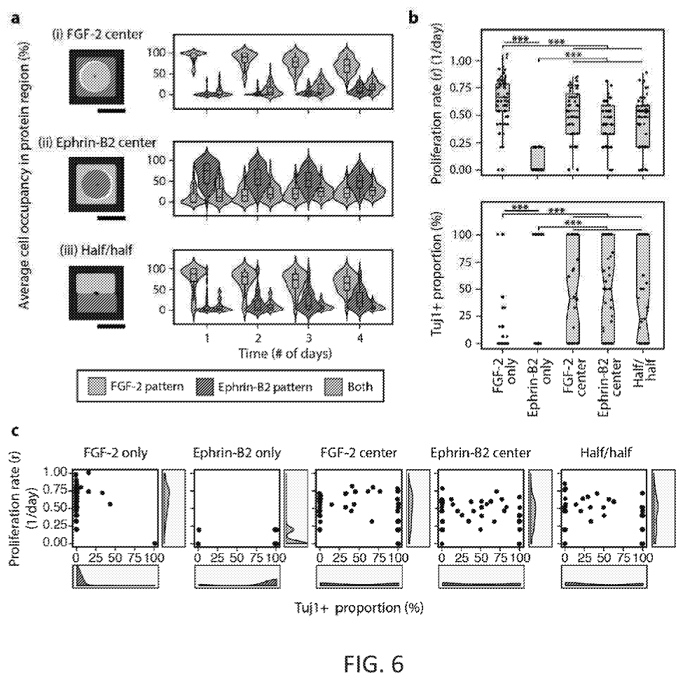

These analytical capabilities were harnessed and how spatial modulation of FGF-2 and ephrin-B2 “domains” shaped the interactions of the single NSC cultures with these two competing niche ligands was assessed. For the FGF-2-center microislands, high average cell occupancy within the FGF-2 domain that persisted over all four days was observed (FIG. 6 a(i)). In contrast, ephrin-B2-center microislands exhibited a much wider distribution of average cell occupancies in the ephrin-B2 region on the first day alone (FIG. 6 a(ii)) and, by the second day, the majority of microislands no longer occupied ephrin-B2 substantially. This observation was further corroborated upon analyzing the half/half microislands, where single NSCs had the freedom to “choose” either protein-patterned region (FIG. 6 a(iii)). Here, average cell occupancy within FGF-2 remained far greater than occupancy within ephrin-B2 or at the interface of both domains, mimicking the FGF-2-center microislands.

As NSC preference for FGF-2 over ephrin-B2 was observed, clonal microislands were then immunostained to investigate whether this cell-occupancy bias translated to cell-fate decisions. A comparison of the three different ligand spatial presentations against FGF-2-center and ephrin-B2-center microislands (FIG. 6 b) revealed that one niche signal did not exclusively dominate over the other. The three different ligand-competition presentations displayed significantly higher proliferation rates over the ephrin-B2-only condition yet were still significantly lower than the FGF-2-only microislands. With regards to differentiation, it was anticipated that the FGF-2 preference would result in cells in these microislands having a more proliferative, stem-like state. However, a surprisingly wide distribution of Tuj1-positive differentiation proportions was observed, including microisland subpopulations spanning both extremes of 100% and 0% neuronal differentiation as well as a few displaying partial neuronal differentiation.

To provide additional insight into this heterogeneity, the relationship between differentiation and proliferation for each of the microisland patterns was further investigated (FIG. 6 c). A clear phenotype for the FGF-2-center microislands in which an increase in Tuj1-positive differentiation correlated with a decrease in proliferation was observed. In the case of the ephrin-B2-center microislands, consistently low levels of proliferation were observed for both high and low differentiation. However, for the microislands containing both ligands in competition, there was no clear trend as microislands exhibiting both high and low differentiation exhibited wide proliferation-rate distributions. Therefore, the observed mix of proliferation and differentiation strongly indicates that these cells are integrating both signals and that the added presence of either cue is insufficient to instruct or completely alter cell-fate decisions, despite NSC's spatial preference toward occupying FGF-2. This outcome highlights the inherent complexity and heterogeneity of NSC behavior and further motivates the need for additional in-depth single-cell analysis to identify potential contributing factors that give rise to this heterogeneity. The unique power of the platform is that it provides the very foundation to do so.

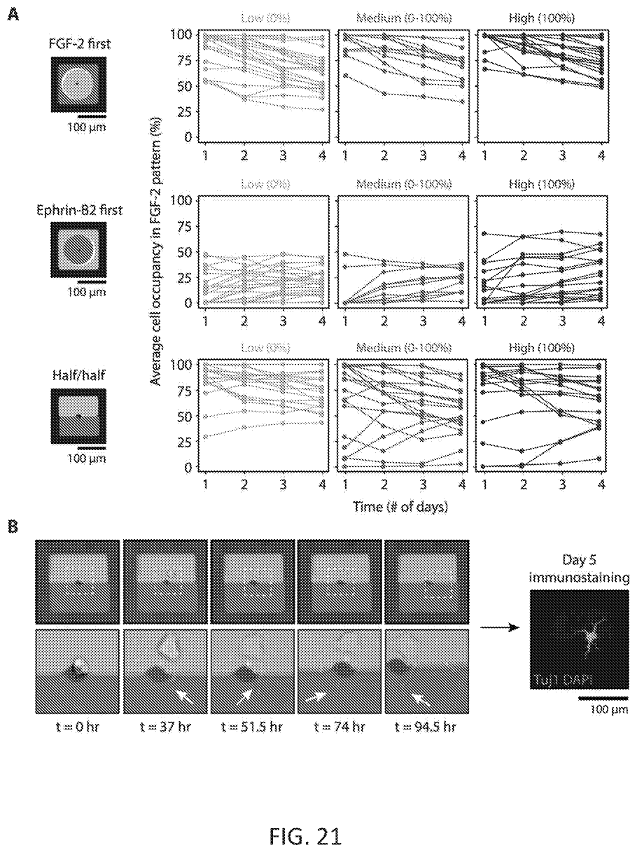

The dynamics of individual microislands was dissected by tracking the changes in average cell-body occupancy within the FGF-2-patterned region over time and microislands were subsequently grouped according to end fate. As shown in FIG. 21 a, each microisland's trajectory is represented by a line. However, rather than identifying unique consensus trajectories for the “Low (0%)”, “Medium (0-100%)”, and “High (100%)” neuronal differentiation bins or a minimum and/or maximum occupancy threshold that could be indicative of cell fate, microislands with similar FGF-2-occupancy patterns spread across all three differentiation categories were observed. More remarkably, instances of single-NSC cultures that underwent differentiation despite having nearly 100% FGF-2 occupancy were observed. A closer examination of these particular cases (FIG. 21 b) revealed a potential source for this paradox, as neurites were visualized extending across into the ephrin-B2 region, such that cells could potential sense and probe both protein patterns throughout differentiation. While it is unclear whether transient sampling of the ephrin-B2 differentiation cue by a short neurite is sufficient to drive a long-term cell-fate decision, future work to track neurite dynamics, which represents an additional computational image analysis challenge, and analyze temporal aspects of cell occupancy within each ligand-patterned region may help elucidate further how single NSCs sense and integrate conflicting instructive cues.

A photolithography-based strategy was introduced to fabricate instructive, multiplexed DNA surface patterns that enable the recapitulation and dissection of complex signaling scenarios in vitro by directing the assembly of heterogeneous cells and/or solid-phase ligands with high spatial precision. The use of patterned photoresist was demonstrated as a template to guide DNA conjugation and coordinate control over multiple DNA strands (i.e. multiplexing) without sacrificing spatial control and resolution. The photoresist masks can be removed and new masks patterned to conjugate oligonucleotide strands with prior DNA layers or patterns retaining their functionality.

The microfabricated DNA patterns were utilized to investigate how NSCs resolve conflicting solid-phase niche cues (i.e. FGF-2 and ephrin-B2) that drive opposing cell fates (i.e. proliferation and differentiation) and how spatial heterogeneity may offer information to direct this decision. By modulating the spatial presentation of these two cues and subsequently tracking average cell-body occupancy within the ligand-patterned regions, a strong bias in spatial occupancy to the FGF-2-patterned regions was discovered.

### Methods

Micropatterning 20-Base Pair (bp) Amine-Terminated Oligonucleotides with Positive Photoresist

Traditional photolithography was employed to pattern aldehyde glass substrates with positive photoresist. S1813 photoresist (Shipley) was spun onto aldehyde-functionalized glass slides (Schott Nexterion) at 3,000 RPM for 30 seconds and subsequently heated for 1.5 minutes on a 100° C. hotplate. Photoresist-coated aldehyde slides were exposed selectively to UV light (365 nm; 260 mJ cm²) with a mask aligner (Karl Suss MJB 3) using a custom mylar mask (Fineline Imaging). Patterns were developed using MF-321 developer (Shipley), washed with 18 mΩ deionized (DI) water, and dried with dry nitrogen gas. Resolution of patterning is limited by the wavelength of light used in the exposure system.

Immediately following photolithography, a 5′-amine-modified, 20-bp oligonucleotide (IDT, Eurofins) solution prepared in 50 mM sodium phosphate buffer (pH=8.5) was dropcast over the photoresist patterns. Slides were covered with a petri dish to prevent evaporation, and the DNA solution was allowed to incubate for 5 minutes. Slides were then heated for 1 hour in a 75° C. oven to induce the formation of Schiff bonds (C═N) between the terminal amine on the DNA and the aldehyde on the glass surface. Slides were then briefly submerged in 0.4% sodium dodecyl sulfate (SDS) in DI water and rinsed with plain DI water to remove excess DNA. To conjugate covalently the DNA strands to the surface aldehyde groups—thereby, converting the hydrolysable Schiff base to single C—N bonds—reductive amination was conducted at room temperature for 15 minutes in 0.25% sodium borohydride (Sigma) in 1×PBS. Upon completion, a second rinse with DI water was performed. To remove the positive photoresist, slides were thoroughly rinsed first with acetone and then DI water, followed by drying with dry nitrogen gas.

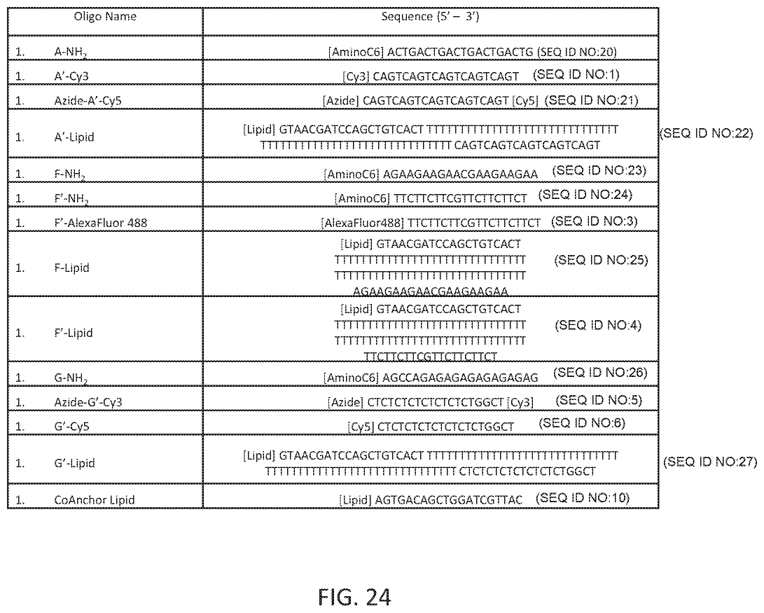

The above steps were repeated to micropattern orthogonal DNA strands (as illustrated in FIG. 1 b), starting with spinning on a new layer of positive photoresist. To align multiple DNA patterns, a microscope with a 10× objective was employed to register fiducial markers on subsequent mylar photomasks to pre-fabricated metal alignment markers on the DNA glass substrate. Completed DNA-patterned slides were stored under vacuum until ready for biopatterning. A complete list of DNA sequences is provided in FIG. 24.

### Patterning Metal Fiducial Markers for Multicomponent DNA Registration

Prior to all DNA patterning, metal alignment markers were fabricated on the aldehyde glass substrate using standard photolithography. Similar to DNA patterning, positive photoresist (Shipley 1813) was photopatterned using a mask aligner (Karl Suss MJB 3) followed by the deposition of a 100 Å thin film of titanium via electron-gun evaporation. Photoresist and excess metal were removed by acetone lift-off. Slides were then washed with DI water, dried with dry nitrogen gas, and stored under vacuum. Precision of DNA pattern registration is limited by lithographic alignment.

Characterizing DNA Patterns with Complementary Fluorescent DNA

Substrates were blocked at room temperature in 2% bovine serum albumin (BSA, Sigma) in 1× phosphate buffered saline, pH 7.4 (PBS) for 1 hour to minimize nonspecific adsorption. Complementary, fluorescently-tagged oligonucleotides were prepared at 0.2 μM in 2% BSA and incubated for 5 minutes on a shaker at room temperature. The substrate surface was then washed 4× with PBS and imaged using an ImageExpress Micro (IXM) high-throughput, automated imager. Complementary oligonucleotide sequences and their conjugated fluorophores are listed in FIG. 24.

Cloning and Expression of Cysteine (Cys)-Terminated Recombinant Proteins in Escherichia coli

The DNA fragments encoding enhanced green fluorescent protein (eGFP) and mCherry were subcloned into a T7 expression vector with a 6×His-tag at the N-terminus to allow for downstream purification and a Cys residue at the C-terminus to enable conjugation with a single-stranded oligonucleotide label. A T7 plasmid containing 6×His-fibroblast-growth-factor 2 (FGF-2)-Cys was a gift from the UC Berkeley QB3 MacroLab facility. All constructs were confirmed via sequencing and subsequently transformed into Rosetta 2 (DE3) competent E. coli cells.

For protein production, 20 mL of an overnight culture was seeded into 1 L of Terrific Broth (TB) supplemented with 100 μg/mL of ampicillin and allowed to grow at standard growing conditions (37° C., 220 rpm) until an OD₆₀₀=0.6. The culture was then induced with isopropyl β-D-1-thiogalactopyranoside (Thermo Fisher Scientific) at a final concentration of 1 mM and allowed to shake for an additional six hours at 30° C. prior to being harvested by centrifugation (5,000 g, 20 min, 4° C.). Bacterial pellets were stored at −80° C. until ready for purification.

### Recombinant Protein Purification Using Gravity Flow Chromatography

Frozen bacterial pellets were thawed on ice and re-suspended in 30 mL of lysis buffer (50 mM NaH₂PO₄, 300 mM NaCl, 10 mM Imidazole, pH=8) supplemented with 1 mg/mL of lysozyme (Sigma), 200 μg/mL of phenylmethylsulfonyl fluoride (Sigma), and 20 mM of 2-mercaptoethanol (Sigma). After incubating on ice for 30 minutes, cells were sonicated for 2 minutes at 60 W (10-sec on/10-sec off) to ensure complete lysis, and cell debris was pelleted via centrifugation (28,000 g, 1 hour, 4° C.). The collected supernatant was purified using gravity flow chromatography with a bed of Ni-NTA agarose (Qiagen). Wash buffer containing 50 mM imidazole was used to remove nonspecific binding of background proteins, and elution buffer containing 250 mM imidazole was applied to the column to elute the His-tagged protein of interest. Elution fractions were separated using SDS polyacrylamide gel electrophoresis (NuPAGE 4-12% Bis-Tris Protein Gel, Thermo Fisher Scientific) and analyzed via Coomassie staining (R-250, Thermo Fisher Scientific). Fractions containing protein of interest were then pooled, and dialysis was performed using a 10 kDa Slide-A-Lyzer cassette (Thermo Fisher Scientific) overnight at 4° C. with 2 solution changes to eliminate excess imidazole as well as desalt the collected protein into storage buffer (1×PBS, 0.5 mM EDTA, 10% glycerol, pH=8). The Pierce BCA Protein Assay (Thermo Fisher Scientific) assay was employed for protein quantification.

Labeling of Cysteine-Terminated Recombinant Protein with Azide-Terminated Oligonucleotide Label Using Dibenzylcyclooctyne (DBCO)-PEG₄-Maleimide Heterobifunctional Crosslinker

Immediately before use, a 10 mM solution of DBCO-PEG₄-Maleimide (Jena Bioscience) was prepared in anhydrous dimethyl sulfoxide (DMSO) and reacted, at a 4-fold molar excess, with the protein-of-interest (i.e. eGFP, mCherry, or FGF-2) diluted to 0.1 mM in Conjugation Buffer (1×PBS with 1 mM EDTA (pH=7)). The conjugation was reacted overnight at 4° C. on a tube rotator. The next day, excess DBCO was removed, and the buffer was exchanged to 1×PBS (pH=7) using a 10 kDa Amicon Ultra-0.5 mL Centrifugal Filter (EMD Millipore). The DBCO-reacted protein-of-interest was then reacted, at a 3-fold molar excess, with an azide-terminated oligonucleotide label overnight at 4° C. on a tube rotator. Reaction efficiency was assessed by running the product on a reducing SDS polyacrylamide gel (NuPAGE 4-12% Bis-Tris Protein Gels, ThermoFisher Scientific) and subsequently imaging the gel using a flat-bed fluorescent scanner (Typhoon 8600, Molecular Dynamics), probing for the fluorescent tag modifying the oligonucleotide label (FIG. 11, FIG. 17). Protein-oligonucleotide conjugate was stored at −20° C. until ready to use.

Labeling of Cysteine-Terminated EphB4-Binding Peptide with Azide-Terminated Oligonucleotide Label Using Dibenzylcyclooctyne (DBCO)-PEG₄-Maleimide Heterobifunctional Crosslinker

Because of its small size, the EphB4-binding peptide (TNYLFSPNGPIARAWC, approx. 2 kDa) (SEQ ID NO:17) was reacted, at a 2-fold excess, with the DBCO-PEG₄-Maleimide (Jena Bioscience) crosslinker, as outlined in the previous protocol for conjugating proteins of interest with an oligo label. The conjugation was reacted overnight at 4° C. on a tube rotator. The resulting DBCO-reacted peptide was reacted again, at a 3-fold molar excess, with an azide-terminated oligo label overnight at 4° C. on a tube rotator. Completion of the reaction was confirmed through visualization of a band shift on a 20% polyacrylamide gel (FIG. 17). Oligonucleotide-labeled peptide was stored at −20° C. until ready to use.

### DNA-Directed Patterning of Oligonucleotide-Labeled Proteins

Similar to the above protocol for characterizing surface DNA patterns with a complementary fluorescent oligonucleotide, substrates were first blocked at room temperature with 2% BSA in PBS for 1 hour to minimize nonspecific adsorption. Complementary, oligonucleotide-labeled fluorescent proteins were prepared at 0.2 μM in 2% BSA and incubated for 5 minutes on a shaker at room temperature. The substrate surface was then washed 4× with PBS and imaged using an ImageExpress Micro (IXM) high-throughput, automated imager.

### Patterning Polyacrylamide (PA) for High-Throughput Single-Cell Cultures Over 5-Day Differentiation

Upon completion of DNA patterning, a PA grid was fabricated onto the substrates to enable clonal analysis of thousands of single-cell cultures over the course of differentiation. This was achieved by first photopatterning a large-scale array of 141 μm×141 μm square features (i.e. “microislands”) arranged with a 200-μm pitch using positive photoresist (Shipley 1813). The photoresist squares were patterned such that the surface DNA patterns were positioned and protected beneath the square features. Subsequently, linear polyacrylamide was dropcast across the substrate surface, reacting with exposed aldehyde groups within unpatterned photoresist areas (FIG. 14). Specifically, a 10% PA solution in 10 mM 4-(2-hydroxyethyl)-1-piperazineethanesulfonic acid) (HEPES, pH=7) was first degassed in a desiccator for 10 minutes to remove dissolved oxygen. Upon activation of the PA solution with 1.5% tetramethylethylenedianine (Bio-Rad) and 0.225% ammonium persulfate (Bio-Rad), 250 μL of the PA mixture was dropcast immediately over the photoresist features, and a Gel Slick (Lonza)-treated glass coverslip was used to spread out the PA solution over the entire DNA-patterned substrate. Following 1 hour of polymerization, the coverslip was removed, and the slide was rinsed with DI water to remove unreacted PA. Finally, the photoresist defining the PA patterns and protecting the DNA were removed by dissolving in acetone. The slide was rinsed with DI water, dried with dry nitrogen gas, and stored under vacuum.

To characterize the nonbiofouling nature of the patterned PA, substrates were incubated with 1 mg/mL of BSA-AlexaFluor 488 conjugate (Thermo Fisher Scientific) at room temperature for 2 hours on a shaker. Loosely bound protein was removed by washing substrates 4× with PBS. Selective protein adsorption to the square microisland features was revealed upon imaging with a FITC filter.

Polydimethylsiloxane (PDMS) Stamping of Flow Cells onto DNA-Patterned Glass Slide

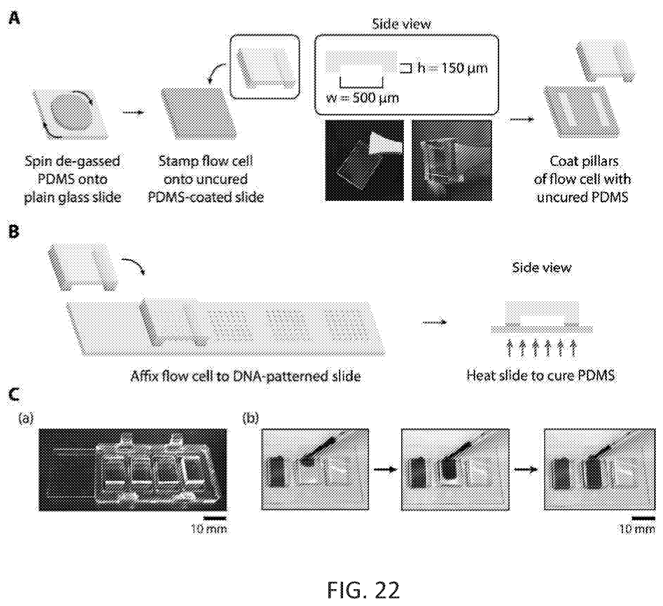

PDMS flow cells were fabricated using standard soft lithography in which a 10:1 Sylgard 184 prepolymer base: curing agent mixture (Dow Corning) was degassed, poured onto a negative silicon master containing 150 μm-high SU8 channels, and cured for 1 hour in an 80° C. oven. Upon complete curing, PDMS flow cells were excised from the negative-relief master using a razor blade and trimmed to fit within one well of a Millicell EZ 4-well chamber (EMD Millipore). To ensure strong attachment of the flow cell during both cell/protein patterning as well as long-term culture, PDMS flow cells were bonded to the DNA-patterned glass substrate using a PDMS stamping protocol (FIG. 22). Briefly, degassed 10:1 PDMS mixture was spin coated onto a blank glass slide at 4,000 RPM for 30 seconds to create a thin PDMS film. The prepared PDMS flow cell was subsequently stamped onto this uncured PDMS film such that the flow cell walls were “inked” with uncured PDMS. The flow cell was subsequently affixed over each well of the DNA-patterned glass substrate. The slide was heated at 65° C. for 1 hour to cure the PDMS “ink”—thus, creating a strong adhesive bond between the flow cell and DNA substrate. Completed slides were stored under vacuum until ready for biopatterning.

### Cell Culture

Adult rat hippocampal neural stem cells (NSCs) were isolated previously from 6-week-old female Fischer 344 rats. To promote monolayer adhesion, NSCs were cultured on polystyrene plates coated with 10 μg/mL poly-L-ornithine hydrobromide (Sigma) in sterile DI water overnight at room temperature and 5 μg/mL of laminin (Invitrogen) in sterile PBS overnight at 37° C. NSCs were maintained in Dulbecco's Modified Eagle Medium/Nutrient Mix F-12 (DMEM/F-12, Invitrogen) with 1% (v/v) N-2 Supplement (Invitrogen) and 20 ng/mL of basic fibroblast growth factor (FGF-2, Peprotech) and incubated at 37° C. and 5% CO₂. NSCs were passaged upon 80% confluency using Accutase (Innovative Cell Technologies). For mixed differentiation studies, NSCs were cultured in normal culture media supplemented with 1% fetal bovine serum (Invitrogen), 1 μM retinoic acid (Enzo Life Sciences), and 1% penicillin-streptomycin (Gibco) in DMEM/F-12+N-2 Supplement. For studies involving protein patterns, NSCs were cultured in maintenance media (DMEM/F-12+N-2) supplemented with 0.1 ng/mL of FGF-2 to promote low proliferation.

### Fluorescent Labeling of NSC Populations Using CellTracker Dyes

NSCs were prepared as a suspension in PBS at 8×10⁶ cells/mL. CellTracker Violet BMQC (2,3,6,7-tetrahydro-9-bromomethyl-1H,5H-quinolizino(9,1-gh)coumarin), CellTracker Green CMFDA (5-chloromethylfluorescein diacetate), CellTracker Red CMTPX, and CellTracker Deep Red (Thermo Fisher Scientific) were added to a final concentration of 2.5 μM, 2.5 μM, 2.5 μM, and 1 μM, respectively, and allowed to incubate with the cells for 15 minutes at room temperature with occasional agitation. To remove excess dye, cells were spun down and re-suspended 3× in 1 mL of PBS. All subsequent steps involving labeled cells were performed in the dark.

Labeling of NSC Membrane with Lipid-DNA

NSCs were detached using Accutase (Innovative Cell Technologies) and prepared at 8×10⁷ cells/mL in PBS. Cells were incubated with 5 μM lipid-DNA for 10 minutes at room temperature and followed immediately by a second incubation with 5 μM of a co-anchor lipid-DNA strand for another 10 minutes to stabilize the first DNA strand. NSCs were then washed 3× via centrifugation at 3000 RPM for 3 minutes with PBS and stored on ice until ready for patterning. Lipid oligo sequences are listed in FIG. 24.

### Single NSC Patterning and Culture

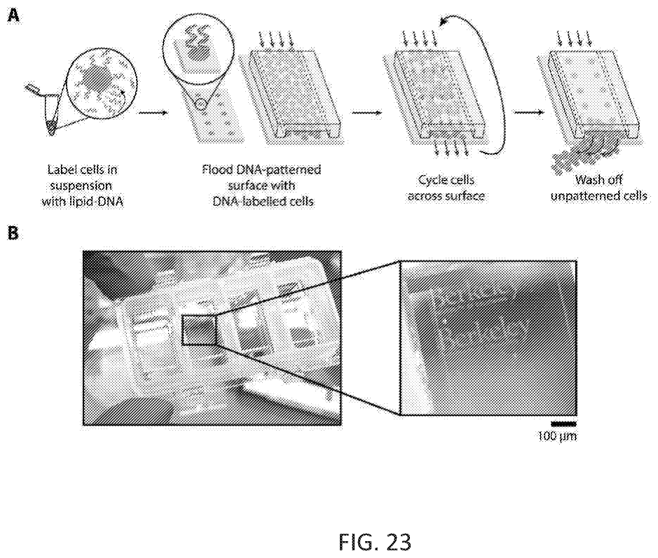

Prior to cell-patterning experiments, DNA-patterned substrates were sterilized in a laminar flow tissue culture hood under ultraviolet light for 15 minutes then blocked with 2% BSA in PBS for 1 hour to minimize non-specific cell attachment. Oligo-labeled NSCs were re-suspended in 2% BSA at 4×10⁷ cells/mL, and 20 uL was injected into the PDM flow cell. The high cell concentration ensured that the entire DNA-patterned area was covered with oligo-labeled NSCs. Cells were then cycled by pipetting 5 uL of the cell suspension into the inlet of the flow channel and removing 5 uL from the outlet. This action was repeated 10-20× to increase the chance of hybridization between the cell-tethered oligos and complementary, surface-tethered oligos. Unpatterned cells were washed away with PBS (FIG. 23). For experiments involving protein patterns, the above steps were repeated with a 0.2 μM solution of the protein(s) of interest in 2% BSA. Upon complete cell and/or protein patterning, 250 μL of the appropriate culture media supplemented with 10 μg/mL laminin was added to each well, and the slide was cultured for 5 days with half media changes (minus laminin) every other day to prevent cells from lifting off of the surface.

### Immunostaining of NSC Differentiation

Following 5 days of differentiation, NSCs were fixed for 5 minutes at room temperature with 4% (w/v) paraformaldehyde and washed 3× for 5 minutes with PBS. Cells were then blocked and permeabilized at room temperature in PBS containing 5% donkey serum (Sigma) and 0.1% Triton X-100 (PBS-DT) for 1 hour prior to being incubated overnight at 4° C. with the primary antibodies, 1:1000 mouse monoclonal IgG for tubulin III (Sigma, T8578) and 1:1000 chicken polyclonal IgG for glial fibrillary acidic protein (Abcam, ab4674), diluted in PBS-DT. The following day, cells were washed 3× for 5 minutes with PBS and incubated in the dark with secondary antibodies, 1:250 Alexa Fluor 488 donkey anti-mouse IgG (H+L) (Thermo Fisher Scientific, A-21202) and 1:250 Cy3 or Alexa Fluor 647 donkey anti-chicken IgG (H+L) (Jackson ImmunoResearch, 703-605-155), diluted in PBS-DT on a shaker at room temperature. Cells were subsequently washed 3× for 5 minutes in PBS with 1:1000 4′,6-Diamidino-2-Phenylindole, Dihydrochloride (DAPI) added during the second wash. Samples were stored in PBS before and during imaging.

### Statistical Analysis

All statistical analysis was performed in MATLAB (R2018a). One-way Analysis of Variance (ANOVA) was used to test for significant differences between variable means. A p-value of less than 0.05 for ANOVA was considered significant. For data with a significant ANOVA result, the Tukey-Kramer method was used to compare between individual groups and test for significance. A p-value of less than 0.05 for the Tukey-Kramer method was considered significant. Details on replicates, ANOVA results, and Tukey-Kramer comparisons are provided in the figure captions.

### DNA-Based Approaches for Temporal Regulation of Ligand Presentation

### Results

### DNase-Induced Cleavage of Single Ligand Presentation

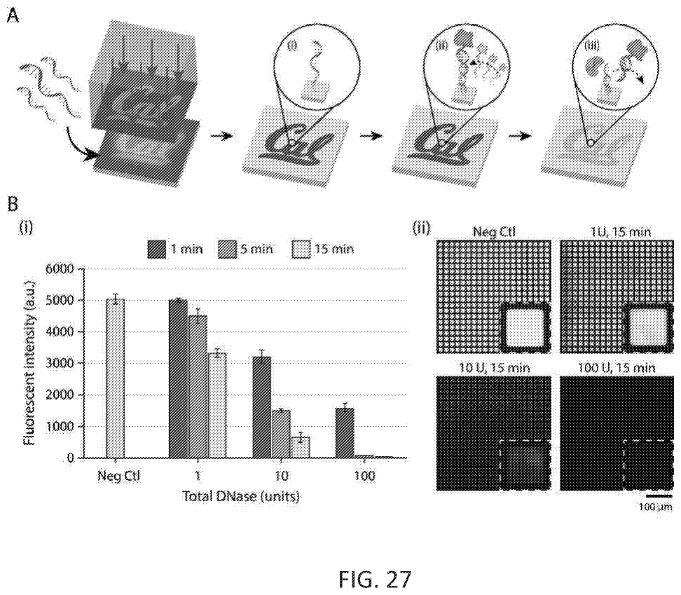

DNA assumes a versatile and powerful role as a nanobiomaterial, storing inheritable information as well as orchestrating RNA and protein production to maintain homeostasis or, in some cases, initiate pathological signaling cascades. More recently, DNA has proven to be useful also as a materials engineering building block due to its ease in programmability, precise nanoscale geometry, and robust hybridization. A range of DNA-based applications have been demonstrated that span nanoscale robots or dynamic delivery constructs generated by DNA origami, DNA-based sensors for diagnostics, molecular characterization tools, and even applications in the field of microelectronics. DNA's unique properties were taken into account, and photolithography was leveraged to pattern multiplexed DNA nanofilms that are highly functional as they assemble and recapitulate complex signaling environments in a controlled, bottom-up manner. Furthermore, inspiration is continuously drawn from biology by utilizing enzymes that recognize and manipulate DNA to strengthen the platform by incorporating the ability to impart temporal control over patterned signaling components. As illustrated in FIG. 27 (a), the first strategy to engineer temporal modulation into the system involves the use of deoxyribonuclease (DNase), which acts by cleaving the phosphodiester linkages of the DNA backbone, releasing DNA-hybridized ligands, and followed by a washing step to remove cleaved products. Due to this enzyme's lack of specificity, this particular approach enables a complete removal of all presented ligands, regardless of patterned DNA sequence. To enable visualization and quantification of DNase-based cleavage kinetics, surface DNA patterns were hybridized to a fluorescent complementary oligonucleotide. Various concentrations of DNase (1 U, 10 U, and 100 U) were introduced; and residual fluorescence was analyzed after various incubation times (1 min, 5 min, and 15 min). As expected, tunable cleavage kinetics were observed by either increasing incubation time or increasing the concentration of DNase (FIG. 27 (b)). Particularly, at 100 units of DNase, rapid cleavage of all patterned DNA within minutes was achieved.

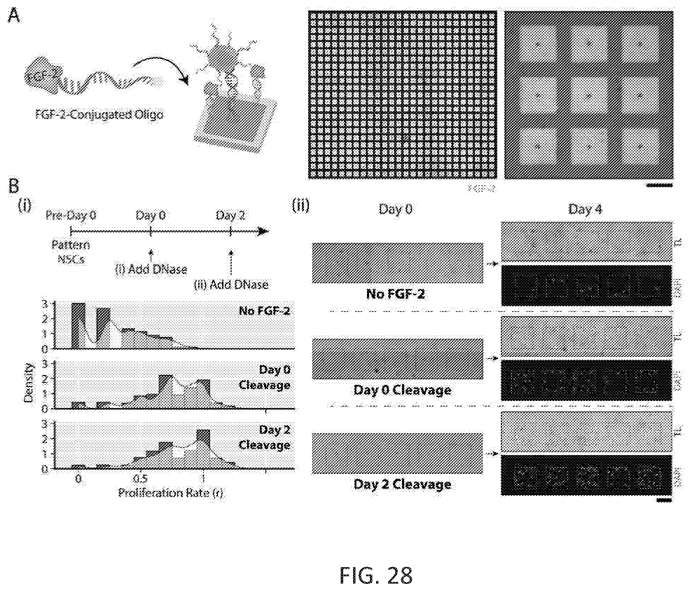

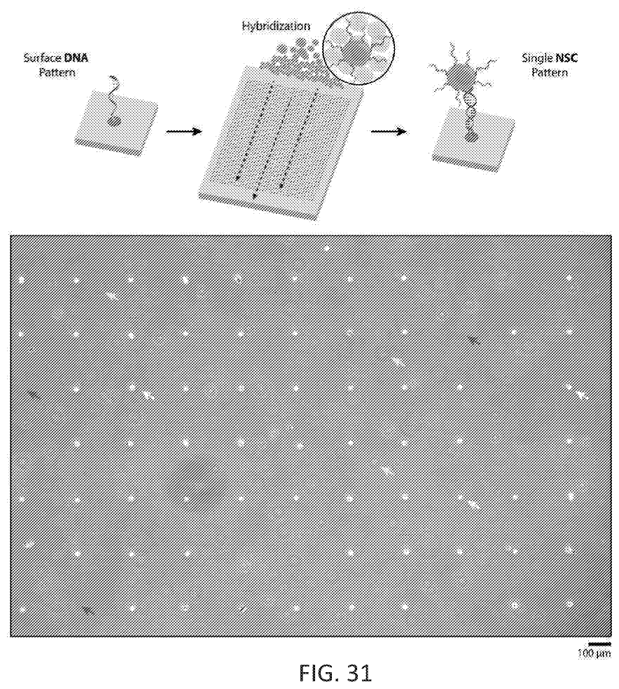

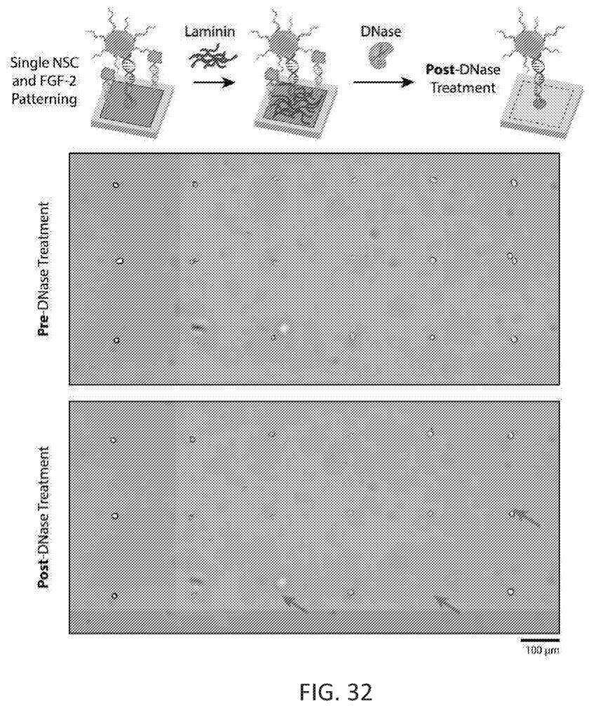

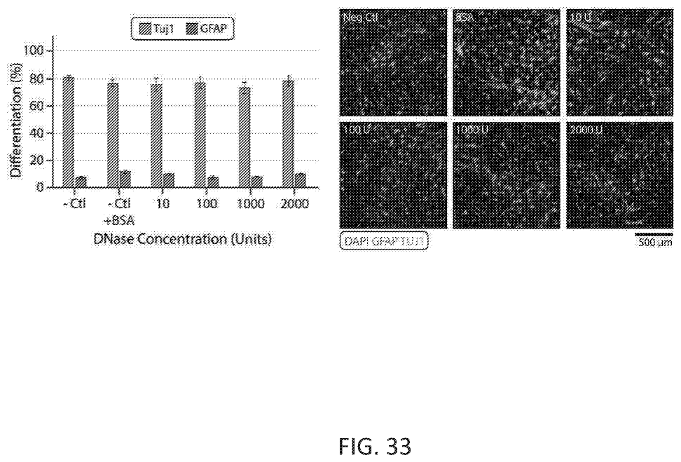

To provide a proof-of-concept demonstration of DNase-based cleavage within a biological system, temporal control over the presentation of fibroblast growth factor-2 (FGF-2) was highlighted, where FGF-2 is a key signaling ligand that promotes proliferation, and different degrees of ligand persistence directed various proliferation rates in NSCs was demonstrated. The DNA-patterning platform was employed to address single NSCs (FIG. 31) within microisland features containing patterned FGF-2. As illustrated in FIG. 28 (a), a fluorescent tag was included at the 3′ end of the oligonucleotide labeling FGF-2 to serve as a readout of protein localization, and the use of photolithography generated a large array of 100's of single-cell/FGF-2 microislands. To achieve temporal control over FGF-2 presentation, DNase was introduced at different timepoints post-cell patterning to vary the persistence of ligand exposure and its effects on single NSC proliferation rate were subsequently assessed. To avoid removing DNA-patterned NSCs upon the introduction of DNase, cells were incubated with laminin for one hour to allow for cells to adhere to the laminin-coated microislands and no longer rely on the DNA-tether (FIG. 32). The removal of FGF-2 at different time points throughout the 4-day culture resulted in a spectrum of proliferation rates (FIG. 28 (b)(i)) that increased from NSCs that lacked patterned FGF-2 to those that had immediate cleavage on Day 0, and cleavage on Day 2. These differences in proliferation rate can also be visualized through DAPI staining (FIG. 28 (b)(ii)). Other key advantages of using DNase include its low cytotoxicity and minimal effect on NSC differentiation (FIG. 33).

Encoding Cleavage Specificity with Restriction Sites

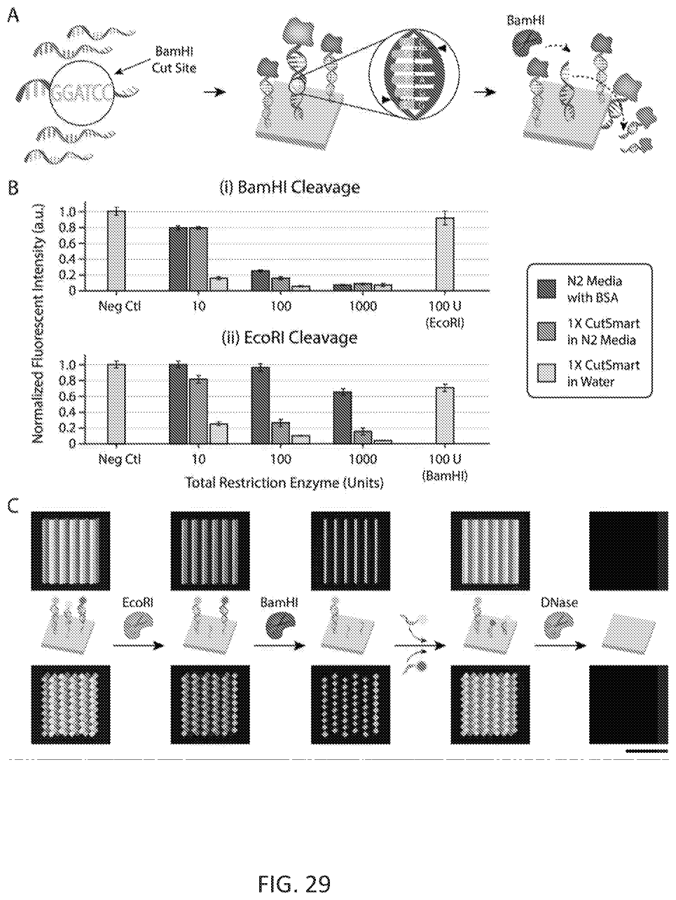

Though the application of DNase achieves temporal control over single DNA-presented ligands, biological systems are often comprised of multiple cues cooperating synergistically or antagonistically. Multicomponent control is required to resolve the temporal parameters governing these complex interactions. The use of orthogonal surface oligonucleotide strands programmed with unique sequences provides a solution for coordinating the DNA-based assembly of multiple signaling ligands. However, in order to obtain cleavage specificity, programming restriction sites into patterned oligonucleotides, as demonstrated in FIG. 29 (a), and subsequently incubating the corresponding restriction enzyme are proposed.

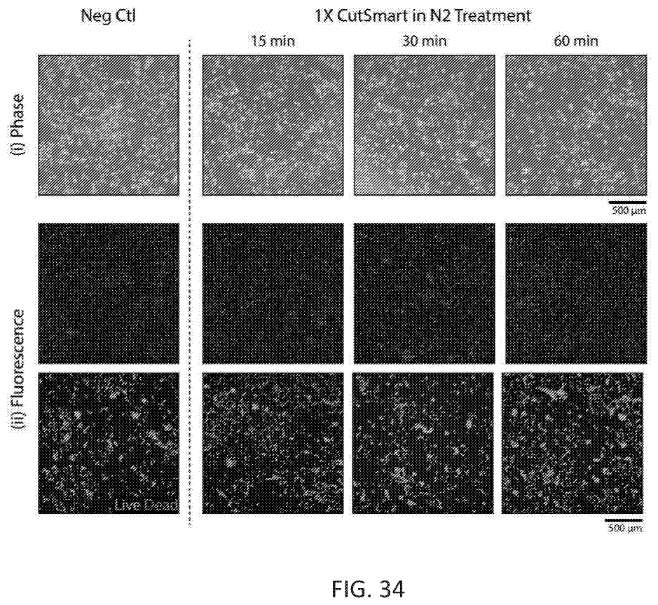

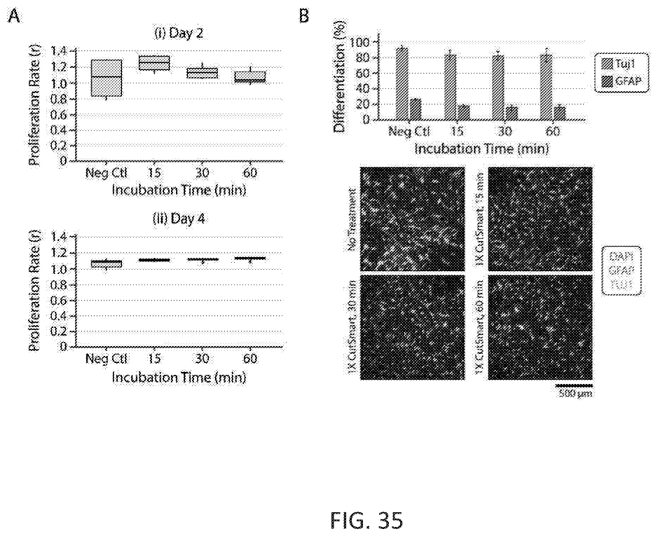

To assess the feasibility of this approach, two DNA strands were designed and tested: one encoded with a Bam HI restriction site (GGATCC) and another with an Eco RI restriction site (GAATTC). Complementary fluorescent oligos were then hybridized and cleavage following incubation with different concentrations of the high-fidelity restriction enzymes was quantified. As highlighted in FIG. 29 (b)(ii), one of the key determinants of cleavage efficiency was the incubation buffer used during cleavage. A comparison of 1× CutSmart buffer, which is commonly employed for molecular cloning, against 1×PBS and NSC media (i.e. N2 media) revealed that CutSmart buffer was far more efficient for both enzymes. However, in the case of high Bam HI concentration at 1000 U, this discrepancy was negligible, as nearly complete cleavage was achieved for all three buffers after a one-hour incubation. Given the discrepancy in cutting kinetics for the different buffers, the effects of incubating 1× CutSmart buffer prepared in N2 media with adult NSCs was investigated. For a one-hour incubation, cell viability remained high (FIG. 34), and there was little to no effect on subsequent NSC proliferation and differentiation (FIG. 35).

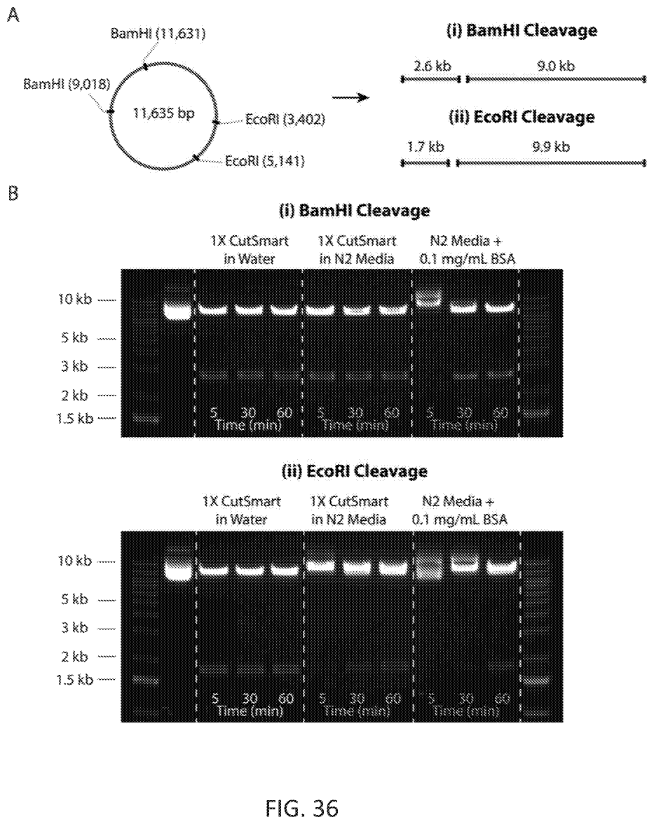

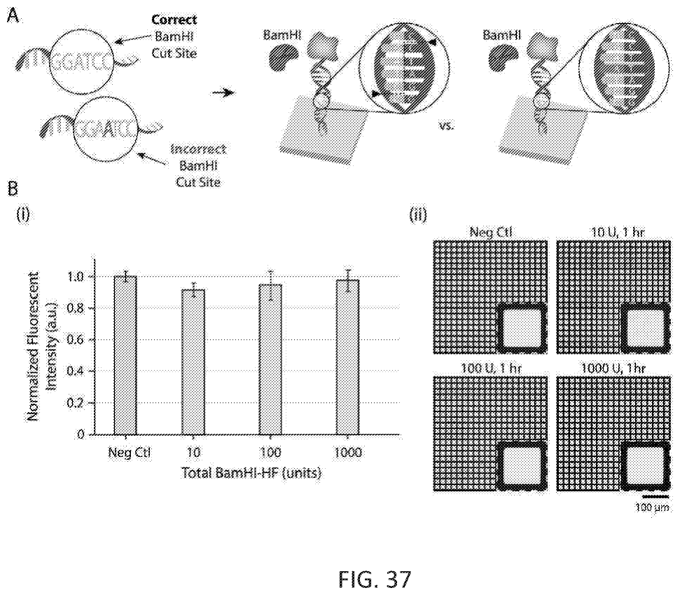

Another key finding from these characterization experiments was the heterogeneity of restriction enzyme cleavage activity. Specifically, Bam HI demonstrated faster cutting kinetics while also demonstrating more tolerance to buffer composition. Eco RI exhibited far poorer cutting in N2 media across the different concentrations—a result most likely attributed to sub-optimal concentration of magnesium, which serves as a necessary co-factor for restriction enzyme activity, and/or the presence of high salt concentration. As shown in FIG. 36, these results were recapitulated in a gel format upon repeating the buffer and concentration conditions using a plasmid as a substrate, one that contained two Bam HI and two Eco RI cut sites. Therefore, when selecting appropriate restriction enzymes and buffer conditions, a rapid gel cleavage screen can be conducted to inform optimal oligo sequence design and ensure appropriate cleavage conditions. The robust specificity of restriction enzymes was highlighted further by testing the addition of Bam HI enzyme to a surface-patterned DNA strand containing a restriction site that closely resembles the correct Bam HI sequence yet deviates by one base pair (FIG. 37). Little to no cutting was detected across different Bam HI concentrations.

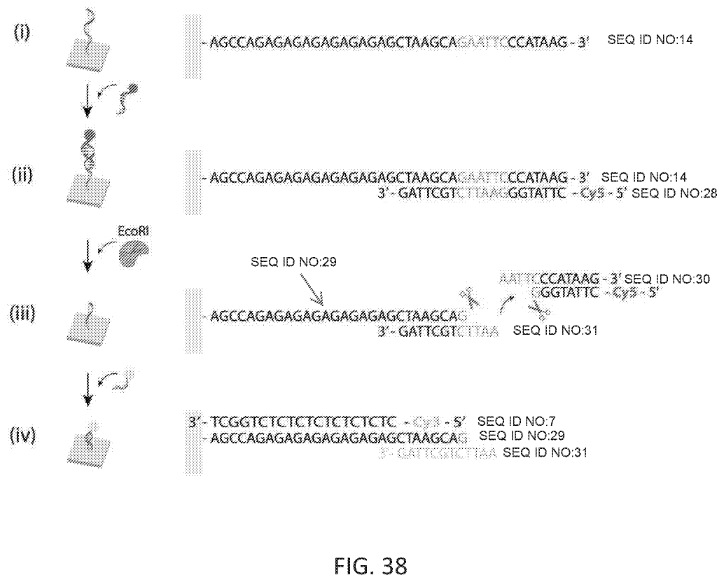

Thus, the strategy of programming restriction sites into patterned DNA strands imparts the necessary specificity to achieve multiplexed temporal control. More complex temporal ligand presentation logic can be achieved by extending the length of the surface-tethered oligonucleotide, such that partial DNA cleavage occurs upon the introduction of a restriction enzyme, leaving a residual 20-bp functional strand that can re-hybridize with a new ligand-conjugated oligo. An example of more complex temporal logic is demonstrated in FIG. 29 (c), where three unique fluorescent oligonucleotides were patterned initially, and the subsequent introduction of Eco RI and Bam HI removed the fluorescent oligos depicted by the yellow and purple patterns, respectively. Both cleavages exposed residual oligo strands that were then hybridized to new complementary strands that inverted the initial fluorophore presentation. Finally, the introduction of DNase removed all DNA-presented fluorophores. In-depth illustration of the DNA sequences employed for this cleavage and re-hybridization approach can be visualized in FIG. 38. The combined use of restriction sites/restriction enzymes with the lithographic DNA-patterning platform provides a systematic and controlled approach to explore the temporal dimension of solid-phase ligand signaling.

A Parallel Approach for Rapid, Specific Cleavage using Streptococcus pyogenes Cas9 (SpCas9) Ribonucleoprotein

The discovery of CRISPR/Cas9 has revolutionized gene-editing therapeutics by enabling targeted Cas9-mediated gene correction. A single guide RNA (gRNA) directs a Cas9 endonuclease to a genomic site of interest, inducing site-specific cleavage that results in disruption of the mutated locus through insertions/deletions (INDELs) or correction via homology-directed repair. Based on the demonstrated specificity and robustness of CRISPR-Cas9, the application of Streptococcus pyogenes (SpCas9) ribonucleoproteins (RNPs) to the DNA patterning system in a parallel approach to the previously described restriction enzyme strategy in the present disclosure to achieve temporal control over patterned ligands was investigated. Analogous to type II restriction endonucleases, which assemble as homodimers that each recognize and cleave within symmetric recognition sequences, spCas9 contains two nuclease domains that together generate double-stranded breaks. Thus, when implemented into the system, Cas9-mediated cleavage releases the DNA-presented ligand that can then be washed away.

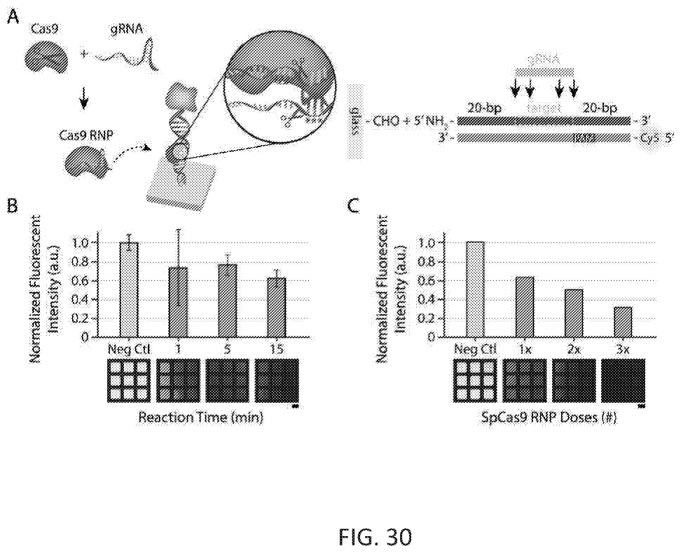

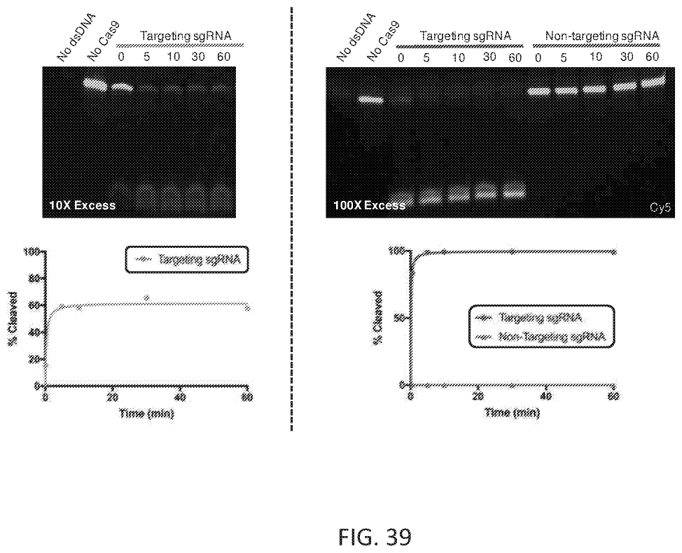

As illustrated in FIG. 30 (a), a single-stranded oligonucleotide amenable to spCas9 cleavage was designed and patterned, where the oligonucleotide contains a 20-bp target sequence located directly adjacent to the SpCas9 protospacer adjacent motif (PAM). 15-bp flanking regions were included to accommodate the bulky ˜160 kDa Cas9 protein, providing both accessibility to the surface-tethered oligo and adequate length for R-loop formation by base-pair hybridization between the gRNA and target DNA sequence. To validate that the assembled Cas9 RNP could target and cleave the designed oligos, cleavage efficiencies in a gel format were first screened, similar to the previously described restriction enzyme strategy in the present disclosure, identifying rapid cleavage for both 10-fold and 100-fold excess of RNP (FIG. 39). The CRISPR oligo was then patterned using photolithography. Upon hybridizing the fluorescent complementary oligo, the kinetics of SpCas9 RNP-mediated surface DNA cleavage were investigated, and incubation times that ranged from 1 min to 15 minutes were tested (FIG. 30 (b)). A cutting saturation effect was discovered—which most likely can be attributed to a lack of SpCas9 RNP displacement post-cleavage. It was also discovered that this saturation was reached within minutes, which highlights the rapid yet titratable action of SpCas9 RNP. This is in contrast to the previous restriction enzyme strategy that demonstrated increased cutting of DNA-presented ligand upon increased incubation times. To achieve various degrees of cutting, an initial proof-of-concept experiment was conducted, where multiple doses at a fixed RNP concentration were tested. As shown in FIG. 30 (c), repeated application of Cas9 RNP (1×, 2×, and 3×) resulted in increased cleavage. Interestingly, preliminary experiments revealed that increasing the RNP concentration does not result in increased cleavage efficiencies. Future work will focus on teasing apart the contributing factors for this observation. Other key experiments include identifying the number of required doses to achieve total cleavage of surface-hybridized DNA, characterizing RNP activity in different biological buffers, and demonstrating CRISPR multiplexing with multiple patterned DNA strands by incorporating orthogonal gRNAs.

The Multiple DNA platform with fabrication of surface DNA patterns comprised of multiplexed oligonucleotide strands which, in turn, assemble heterogeneous ligands through the hybridization between surface-tethered oligos and ligands labelled with the unique, complementary oligos. With DNase, it was demonstrated that nonspecific yet rapid, one-pot cleavage of hybridized ligands, was achieved for controlling solid-phase ligand persistence of a single ligand and investigating the effects of timing and strength/duration on cell behavior. For signaling environments in which multiple signals are operating together, both restriction endonucleases and SpCas9 RNP's can be utilized to achieve cleavage specificity or multiplexed temporal control.

### Methods

### Surface DNA Patterning Using Photolithography

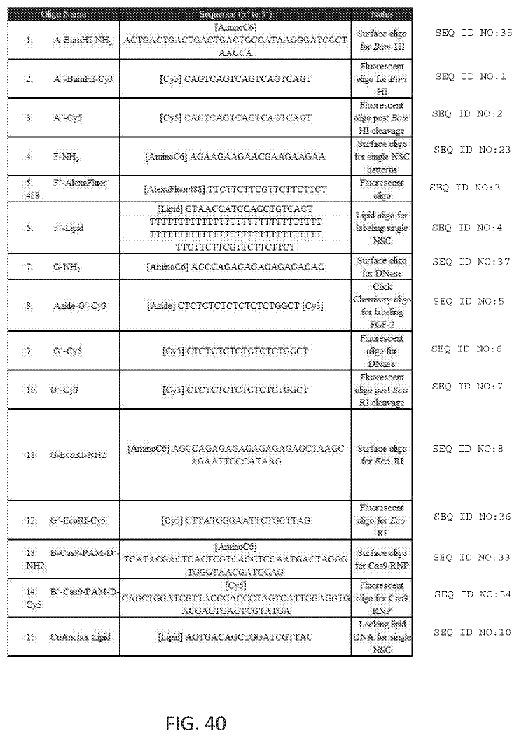

20 base-pair, single-stranded oligonucleotides (IDT) were patterned onto an aldehyde glass slide (Schott Nexterion). In short, microfabricated positive photoresist (Shipley S1813) served as a physical mask, selectively exposing aldehyde surface regions and subsequently guiding the conjugation of amine-terminated oligonucleotides with high spatial control. An oligonucleotide solution prepared in 50 mM sodium phosphate buffer (pH=8.5) was dropcast over the patterned photoresist, heated to induce condensation, and treated with 0.05% sodium borohydride in 1×PBS for 10 minutes to conjugate covalently the amine-terminated oligonucleotide to the aldehyde substrate. Photoresist was removed by rinsing slides with acetone, followed by DI water, and dried under a dry nitrogen stream. Slides were stored under vacuum until ready to use. DNA sequences are provided in FIG. 40.

To visualize patterns, Millicell EZ 4-well chambers (Millipore) were first secured onto the glass slide, and the surface was blocked for 1 hour on a shaker by adding 1 mL of blocking buffer (2% bovine serum albumin (BSA) in 1×PBS) to each well. The 2% BSA was exchanged using a pipette with 250 μL/well of a 0.2 μM solution comprised of the complementary oligonucleotide containing a fluorescent tag prepared in the same blocking buffer. Following a 5-minute incubation on a shaker to allow for hybridization, wells were rinsed 4× with 1×PBS to remove unbound fluorescent oligonucleotides and stored in fresh 1×PBS during imaging.

### Cell Culture

Adult rat hippocampal neural stem cells (NSCs) were isolated previously from 6-week-old female Fischer 344 rats. To promote monolayer adhesion, NSCs were cultured on polystyrene plates coated with 10 μg/mL poly-L-ornithine hydrobromide (Sigma) in sterile DI water overnight at room temperature and 5 μg/mL of laminin (Invitrogen) in sterile PBS overnight at 37° C. NSCs were maintained in Dulbecco's Modified Eagle Medium/Nutrient Mix F-12 (DMEM/F-12, Invitrogen) with 1% (v/v) N-2 Supplement (Invitrogen) and 20 ng/mL of basic fibroblast growth factor (FGF-2, Peprotech) and incubated at 37° C. and 5% CO₂. NSCs were passaged upon 80% confluency using Accutase (Innovative Cell Technologies). For differentiation studies, NSCs were cultured in normal culture media supplemented with 1% fetal bovine serum (Invitrogen), 1 μM retinoic acid (Enzo Life Sciences), and 1% penicillin-streptomycin (Gibco) in DMEM/F-12+N-2 Supplement. For studies involving protein patterns, NSCs were cultured in maintenance media (DMEM/F-12+N-2) supplemented with 0.1 ng/mL of FGF-2 to promote low proliferation and differentiation.

### DNA-Based Single NSC Patterning

High-throughput capture of thousands of single NSCs is achieved by first labeling cell membranes with lipid-conjugated oligonucleotides containing the complementary sequence to surface-patterned, 15 μm-diameter DNA spot features. The prepared DNA-tethered cell suspension is then flowed 10-20× across the DNA substrate with the aid of a polydimethoxysilane (PDMS) flow chamber to promote hybridization between surface-bearing and cell-labeling oligos—thus, achieving cell capture. Both the cell-labeling process and flow steps are described previously in detail.

### DNase-Based Cleavage Experiments

Deoxyribonuclease I (DNase) (Worthington, LS002138) was supplied by the manufacturer as a lyophilized power and was re-suspended in NSC media (DMEM/F-12+1% N-2 Supplement) at 1,000 Kunitz units/mL. For DNase-cleavage characterization experiments, stock DNase was diluted further to the appropriate Kunitz units in 250 μL of media and subsequently incubated at 37° C. within each DNA-patterned well for the designated amount of time. Wells were then rinsed 4× with 1×PBS to remove cleaved DNA.

For DNase-cleavage experiments of DNA-hybridized fibroblast-growth factor 2 (FGF-2), patterned single NSCs were first incubated with 20 μg/mL of laminin for 1 hour at 37° C. to ensure that cells no longer rely on DNA to be tethered to the surface and, thus, not removed upon the introduction of DNase. 1000 units of DNase was then introduced to cells by pipetting into the PDMS flow cell and allowed to incubate for 15 minutes at 37° C. Wash steps were conducted by cycling PBS into and out of the flow cell 10×. A total of three DNase treatment was conducted to ensure complete cleavage of patterned FGF-2 and validated upon imaging as an observed loss of Cy3 signal.

### Restriction Enzyme-Based Cleavage Experiments

For DNA-patterning experiments, high-fidelity Bam HI and Eco RI (NEB) were prepared in the following buffers: 1× CutSmart (NEB) diluted in water, 1× CutSmart diluted in NSC media (DMEM/F-12+1% N-2 Supplement), and/or NSC media supplemented with 0.1 mg/mL bovine-serum albumin (BSA). 250 μL of prepared restriction enzyme solution containing the desired units and buffer were then added to fluorescently-hybridized DNA-patterned slides, prepared as described above.

For restriction enzyme digest characterization experiments, 20 units of high-fidelity Bam HI and Eco RI were prepared in the aforementioned buffers and incubated with 500 ng of a 11.6 kb plasmid containing two Eco RI cut sites and two Bam HI cut sites in a total volume of 50 μL for 5 min, 30 min, or 60 min at 37° C. Digest efficiency was assessed by running products on a 1% Tris-Acetate-EDTA (TAE) agarose gel, and DNA bands were visualized using SyberSafe (Thermo Fisher Scientific).

Streptococcus pyogenes Cas9 (SpCas9) Ribonucleoprotein (RNP)-Based Cleavage Experiments

To assemble the RNP, recombinant SpCas9 was diluted to 10 μM in RNP buffer (20 mM Tris-HCl, 100 mM KCl, 5% (v/v) glycerol, 5 mM MgCl₂, 1 mM TCEP, 50 μg/mL, pH=7.5) and added to 12 μM of folded single guide RNA (sgRNA) prepared in folding buffer (20 mM HEPES, 150 mM NaCl, pH=7.5). The two components were incubated at 37° C. for 10 minutes and subsequently diluted further in RNP buffer at the desired concentration prior to being added to DNA-patterned substrate.

FIG. 1 shows high-resolution surface DNA patterning using photolithography. (a) Multicomponent patterns of unique 20 base-pair oligonucleotides instruct the spatial organization of cells and ligands through the hybridization between surface-presented oligonucleotides and complementary oligonucleotide-labeled biological components. (b) Surface DNA patterns are fabricated through the successive utilization of, first, photolithography to define regions of reactive aldehyde groups for oligonucleotide conjugation (Step 1) and, second, a reductive amination step to react covalently the amine-terminated oligonucleotides to the aldehyde-functionalized glass surface (Step 2). Multicomponent DNA patterns are assembled by patterning a new layer of positive photoresist and repeating steps 1 and 2 using unique oligonucleotides. (c) The use of photolithography enables the fabrication of high-resolution, spatially complex DNA patterns. Patterned photoresist is utilized as a mask to conjugate selectively amine-terminated oligonucleotides, which can be visualized by hybridizing a complementary fluorescent oligonucleotide. (d) A dynamic range of surface DNA pattern intensities (left) can be achieved by tuning the DNA solution concentration. Representative fluorescent intensity profiles (right, top) and their corresponding images of a 40-μm DNA spot array (right, bottom) are illustrated for a low (0.5 μM), medium (5 μM), and high (50 μM) DNA concentration. Error bars are standard deviation and n=3. All scale bars represent 100 μm.

FIG. 2 depicts microfabricated DNA patterns direct the capture of NSCs. (a) Patterned surface oligonucleotides organize a fluorescently-labeled population of NSCs with high spatial precision through Watson-Crick base pairing between the surface-conjugated DNA and the temporary lipid-modified DNA tethered to the cell membranes. (b) Cell-capture efficiency of 20 μm-diameter DNA-spot patterns was dependent upon the concentration of DNA solution with a significant drop of efficiency occurring at a concentration below 5 μM (left). This is seen in the representative NSC-patterned images of a 10×10 array of 20 μm-diameter DNA spots for a range of concentrations (right). (c) The number of DNA-captured cells can be controlled by tuning the feature size of the DNA patterns. Representative images (left) demonstrate DNA spots with different diameter dimensions capturing varying numbers of fluorescently-labeled NSCs. Moreover, an increase in diameter size of DNA-patterned spots (right) results in an increase in cell-capture number (blue dots) that follows a similar increasing trend in spot area (yellow line). All error bars are standard deviation and n values are reported in Table S3. Scale bars represent 500 μm.

FIG. 3 depicts scalable, multicomponent DNA patterns organize heterogeneous cell populations. Characterization of multiple fabrication steps highlight the compatibility of photolithography with DNA patterning. (a) The integrity of surface DNA patterns is preserved—as indicated by the ability to hybridize with its complementary, fluorescent oligo counterpart—when subjected to repeated photolithographic fabrication steps, as would occur when patterning multiple DNA layers (i.e. removal of photoresist (PR) with acetone and patterning of a new layer). Despite a slight initial drop upon the application of a second PR layer, the average fluorescent intensity of DNA-patterned features remains robust upon a third and fourth photolithography step. (b) The functionality of the surface-modified aldehyde groups, which is necessary for DNA conjugation, is also preserved during successive PR layer applications. Additional photolithography steps yield surface DNA patterns with robust fluorescent intensities. Scale bars represent 100 μm. All error bars are standard deviation and n=3. (c) Micron-scale registration of 3 complex DNA patterns were patterned and visualized with unique complementary fluorescent oligonucleotides. (d) To highlight their functionality, multicomponent DNA patterns assembled three distinct, fluorescently-tagged NSC populations with high spatial control and specificity by labeling each population with unique complementary, lipid-modified oligos that insert into the cell membrane. Scale bars represent 500 μm.

FIG. 4 depicts microfabricated DNA patterns direct the spatial organization of solid-phase ligands. (a) The heterobifunctional linker, dibenzyocyclooctyne (DBCO)—polyethylene glycol (PEG4)—maleimide, enables covalent labeling of ligands of interest with an oligonucleotide label. A free sulfhydryl group on the protein is reacted first with the maleimide moiety on the crosslinker, introducing a DBCO functional group to the ligand that then reacts via click chemistry to an azide-terminated oligonucleotide. (b) The incorporation of an oligonucleotide label possessing a fluorescent tag enables imaging and monitoring of DNA-directed ligand patterns. For proof of concept, enhanced green fluorescent protein (eGFP) with a Cy5 tag was assembled using DNA surface patterns (top). Trends in fluorescent intensity profiles for the patterned protein and the fluorescent tag closely matched one other when tuning surface DNA concentrations, suggesting that the fluorescent oligonucleotide label can also be employed as a relative readout of patterned protein concentration (bottom). (c) Multicomponent DNA surface patterns enable tunable control over each ligand concentration as evident in the DNA assembly of eGFP and mCherry. mCherry concentration was held constant as eGFP concentration was tuned as quantified by the change in eGFP fluorescence intensity (bottom) and visualized in the fluorescent composite images (top). Scale bars represent 500 μm.

FIG. 5 depicts multicomponent DNA patterns enable tight spatial control and investigation of the presentation of competing ligand cues, FGF-2 and ephrin-B2, on single NSC behavior. (a) Overview of two 4-layer DNA patterning schemes that direct the assembly of FGF-2, ephrin-B2-mimetic peptide, single NSCs, and PA patterns (left). (i) The top PR patterns segregate each ligand to one half of the microisland, exposing the patterned single NSC to both solid-phase cues equally, while (ii) the bottom PR patterns forces the presentation of one ligand over the other. A representative image (right) of a large-area microisland array containing both presentation strategies; the patterned ligands are visualized by their respective fluorescent oligonucleotide labels (FGF-2 in cyan and ephrin-B2 in magenta). The zoomed-in insert highlights the simultaneous assembly of three different spatial presentation configurations: half/half, FGF-2 center, and ephrin-B2 center. (b) Representative time-lapse images illustrating cell proliferation and migration for 3 sample microislands of the different ligand spatial configurations and their corresponding day 5 immunostaining results: (i) FGF-2 center, (ii) ephrin-B2 center, (iii) half/half. (c) Quantification of cell body counts within FGF-2 (cyan) and ephrin-B2 (magenta) patterns over 4-day time-lapse utilizing custom analysis script, corresponding to the same 3 sample microislands in part (b). All scale bars represent 100 μm.

FIG. 6 shows cell occupancy in response to various ligand presentations of FGF-2 and ephrin-B2 and resulting end fate after 5-day differentiation. (a) Average cell occupancy of cell bodies within the FGF-2 (cyan), ephrin-B2 (magenta), and spanning both (grey) protein-patterned regions were tracked over time for each of the three ligand spatial presentations: (i) FGF-2 center, (ii) ephrin-B2 center, and (iii) Half/half. A strong spatial bias towards FGF-2 was observed. (b) Analysis of end fate through quantification of proliferation (top) and neuronal differentiation (bottom) reveal that, despite spatial preference towards FGF-2, some NSC microislands integrated both signals, generating significant heterogeneity. (c) The potential identification of distinct subpopulations that give rise to this heterogeneity was tested by comparing proliferation rate vs. Tuj1-positive differentiation on a per microisland basis. However, the heterogeneity in single NSC response to the presentation of both ligands was further highlighted as permutations of high/low differentiation and proliferation were present for all three competing ligand presentations. n=55 for each ligand presentation. All p-values obtained from Tukey-Kramer test. ***p<0.001. All scale bars represent 100 μm.

FIG. 7 shows characterization and optimization of DNA Patterning Steps. (A) Surface patterning of amine-terminated oligonucleotides is strongly influenced by the composition of the buffer used to dilute the DNA. To visualize and characterize resulting DNA patterns, the fluorescent signal from a complementary fluorescent oligonucleotide that had been flowed across, and hybridized onto, the surface DNA patterns is imaged and quantified. The use of water as the dilution buffer resulted in a weak fluorescent signal. Adding salt to the buffer improved the fluorescence intensity of the patterns; however, an excess of salt, e.g. 1×PBS buffer, resulted in undesirable background signal. Saline-sodium citrate (SSC) buffer, commonly employed for DNA microarray technologies, partially dissolved the positive photoresist, contributing to even higher background fluorescence. Thus, the ideal buffer is one that 1) incorporates low salt concentration and 2) preserves the integrity of the patterned photoresist. In the reported experiments, 50 mM sodium phosphate buffer (pH=8.0) was determined to be the ideal candidate for generating robust DNA patterns with minimal background. Scale bars represent 500 μm. (B) Two key steps in the DNA-patterning process were investigated further. For the aldehyde-amine condensation step, various (a) oven temperatures (25° C., 50° C., 75° C., 100° C.) were tested for a 30-min incubation as well as a range of (b) incubation times (5 min, 15 min, 30 min, and 1 hour) at 75° C. Results from both highlight the necessary balance in selecting an appropriate temperature/time to ensure complete condensation. At the highest temperature (i.e. 100° C.), increased coffee-ring effect with higher DNA concentrations accumulating at the edge of the dropcast DNA was observed; thus, a condensation temperature of 75° C. and incubation time of 30 minutes were selected. (C) For the reductive amination step, all tested incubation times (1 min, 5 min, 15 min, and 30 min) generated high-intensity DNA patterns. Longer incubation times, however, resulted in a slight drop in intensity values. Error bars represent standard deviation and n=4.

FIG. 8 shows optimization of DNA-Patterned Features for High Efficiency Single-Cell Capture. DNA spot features were microfabricated onto a surface to capture single adult neural stem cells labeled with a lipid-oligonucleotide containing the complementary sequence. (A) To achieve high efficiency single-cell capture, a combination of DNA-patterned spot size diameters and DNA concentrations were screened, where the screening identified a 15 μm-diameter spot size and 20 μM DNA concentration as the optimal condition. (B) Representative images of efficient and inefficient single-cell capture of fluorescently-labeled adult neural stem cells by photolithographic surface DNA patterns.

FIG. 9 shows Re-Use of Photoresist (PR) Layer vs. New PR Layer for Multicomponent DNA Patterning. (A) Two strategies were investigated for fabricating multicomponent surface DNA patterns. The (a) first strategy involved re-using the same PR layer that was employed to pattern the first DNA layer to conjugate a second oligo. The (b) second strategy involved removing the first PR layer upon the successful conjugation of the first oligo and applying a new PR layer on which to perform photolithography to define surface patterns for a second oligo. (B) Both strategies were capable of generated two-component DNA surface patterns as illustrated in the composite images (right). However, the (a) first strategy resulted in undesired contamination of the first DNA layer pattern with the second oligonucleotide (red arrows), most likely a result of the second oligo reacting with residual unconjugated aldehyde groups from the first DNA pattern region. In the case of the (b) second strategy, distinct and separate patterns with no bleed through of fluorescent signal between the layers was achieved.

FIG. 10 depicts robustness of Photolithographic Approach for Assembling Multiplexed DNA Patterns. The repeated application of multiple patterned photoresist layers was tested for up to 10 DNA layers. Hybridization with a complementary fluorescent oligonucleotide revealed that each layer retained its functionality.

FIG. 11 shows tunable Multicomponent DNA Patterns within the Same and Distinct Layers. Surface DNA concentrations can be controlled for multiple DNA components when patterned (A) within the same layer as demonstrated by the two-component, mixed bear patterns in which the concentration of one DNA strand (i.e. DNA 1) is held constant while a second strand (i.e. DNA 2) is tuned across (a) high, (b) medium, and (c) low concentrations, or (B) across separate layers as each layer contains a unique oligonucleotide and, similarly, one layer (i.e. DNA Layer 1) is held constant while the concentration of the other (i.e. DNA Layer 2) is varied across (a) high, (b) medium, and (c) low concentrations.

FIG. 12 shows characterization of Labeling Reaction of Recombinant Fluorescent Protein, eGFP, with a Fluorescently-Tagged Oligonucleotide Label using Dibenzocyclooctyne (DBCO) Heterobifunctional Cross-linker. eGFP was produced and purified from E. coli and subsequently reacted with a fluorescent oligonucleotide label to enable DNA-directed solid-phase ligand patterning. (A) eGFP was cloned with the following terminal tags: 1) histidine tag at the N-terminus to enable purification using a Ni-NTA column and 2) cysteine tag at the C-terminus to enable oligonucleotide conjugation via click chemistry. The two-step labeling reaction involved first reacting the fluorescent protein with a 4× molar excess of the heterobifunctional cross-linker, DBCO-PEG₄-maleimide, forming a stable thioether linkage between the maleimide group on the cross-linker and the sulfhydryl group on the C-terminal cysteine of the protein. The second step then involved reacting the DBCO-labeled protein at a 3× molar excess with the fluorescent oligo label via the terminal azide on the oligo and the DBCO moiety on the protein. (B) SDS-PAGE was utilized to assess the reaction efficiency. Coomassie staining of eGFP revealed a product band shifted ˜7 kDa, corresponding to the molecular weight of the fluorescent oligonucleotide. To validate that this product was labeled with DNA, the gel was imaged for fluorescence as the oligonucleotide label possesses a Cy5 fluorescent tag at the 3′ end. A fluorescent band with a molecular weight matching the Coomassie-stained bands was detected and, importantly, no fluorescent band was detected at ˜7 kDa, suggesting that all of the oligos were conjugated to eGFP.

FIG. 13 depicts Stability and Specificity of DNA-Directed Enhanced Green Fluorescent (eGFP) Protein Patterns. (A) To characterize the stability of DNA-directed ligand patterns for use in long-term differentiation studies, an oligonucleotide label was conjugated to recombinant eGFP, and the conjugate was hybridized to its complementary, surface-patterned DNA. eGFP patterns were monitored over the course of a 6-day time-course with conditioned media (C.M.) changes occurring every other day. The fluorescent intensity emitted from eGFP was quantified on Days 0 and 6 (left). Despite a slight decrease in intensity, eGFP patterns continued to be well defined and stable, as illustrated in the representative images on days 0, 2, 4 and 6. Error bars are standard deviation and n=3. (B) To highlight the specificity of DNA-directed ligand patterning, a combination of patterning controls was tested using eGFP as the ligand of interest. No eGFP signal was detected for conditions in which there was (a) an absence of an oligonucleotide label conjugated to eGFP or (b) noncomplementary surface-patterned DNA. Only when (c) an oligonucleotide label on the eGFP is complementary to the surface DNA patterns was there detectable eGFP fluorescent patterns.

FIG. 14 depicts optimization of Polyacrylamide (PA) Patterning using Photolithography. In addition to directing the spatial assembly of DNA onto a substrate, patterned positive photoresist was used to fabricate PA grids, generating an array of cell-contained 141 μm×141 μm microislands. Each microisland accommodates a single adult neural stem cell (NSC) and enables high-throughput clonal analysis. The PA grid restricts each patterned single cell to within a microisland over the course of differentiation. (A) To achieve high-fidelity, non-biofouling PA patterns, various combinations of PA percentages and laminin concentrations were tested by seeding 50,000 NSCs and observing the integrity of cells patterned within the micro-island features over the course of 6 days. A low percentage of PA (i.e. 5% PA) was insufficient to retain NSCs within the microislands across all of the different laminin concentrations tried. A 10% PA composition with low 10 μg/mL laminin concentration demonstrated non-biofouling properties for the first two days but, by days 4 and 6, NSCs were observed escaping the microisland features. Although 20% PA resulted in robust non-biofouling grids for all laminin concentrations for up to 6 days, washing off excess PA was difficult. Thus, 10% PA was optimized further by (B) testing the reaction time of the persulfate-induced grafting of the linear PA to the surface aldehydes. A minimum reaction time of 1 hour was necessary to retain cells within the microislands over the course of 6 days. This condition was utilized for all subsequent experiments.

FIG. 15 depicts microfabricated DNA and PA Patterns Support High-Throughput Clonal Analysis of Adult Neural Stem Cells (NSCs). (A) An array of 15 μm-diameter DNA spots (top left, blue circles) was microfabricated in combination with PA patterns (top left, green) to enable, first, the high-throughput capture of oligo-labeled single NSCs and, second, long-term biological studies of 1000's of single-cell cultures in parallel. Compatibility of DNA and PA patterning was evident as DNA patterns could be visualized with a complementary fluorescent oligo (magenta), while PA grid patterns were evident upon incubation with bovine serum albumin (BSA)-AlexaFluor488 as the PA is non-biofouling and excluded BSA to square microisland features. A representative grid of DNA-directed single NSC patterns are evident (right). (B) The combination of PA and DNA patterns enabled NSC clonal analysis following 5-day treatment with various soluble media conditions. (a) As anticipated, 20 ng/mL of FGF-2 promoted high proliferation with low levels of differentiation. As FGF-2 concentration decreased, proliferation rates also decreased as neuronal differentiation increased, while mixed differentiation containing 1% fetal bovine serum and 1 μM retinoic acid exhibited the highest neural differentiation as visualized by the high Tuj1 expression in the (b) representative immunostaining images of microislands for the different soluble conditions. Error bars are standard deviation and n values can be found in Table S3. All p-values obtained from Tukey-Kramer test. ***p<0.001. All scale bars represent 100 μm.

FIG. 16 shows purification of Recombinant Fibroblast Growth Factor-2 (FGF-2) and 5-Ethynyl-2′-Deoxyuridine (EdU) Pulse-Chase Experiment to Validate Protein Activity in Adult Neural Stem Cells (NSCs). (A) A T7 vector containing the ectodomain of human FGF-2 with a N-terminal histidine tag and a C-terminal cysteine was a gift from University of California—Berkeley Macrolab and subsequently cloned and purified from Rossetta2 E. coli (orange arrow highlights Coomassie band corresponding to purified FGF-2). (B) To validate the bioactivity of the aforementioned recombinant cysteine-terminated FGF-2, an EdU pulse-chase experiment was conducted with NSCs, comparing the proliferation of cells treated with recombinant FGF-2 versus commercially-available FGF-2 commonly used for stem cell maintenance. Cells were plated at 15,000 cells/cm², starved from FGF-2 for 24 hours to deplete residual FGF-2 added during NSC subculture, treated with either commercial or recombinant FGF-2 for 24 hours, pulsed with 1 uM EdU for 8 hours, fixed/stained, and imaged. Recombinant FGF-2 induced similar proliferation in comparison to commercial FGF-2 at both high (20 ng/mL) and low (0.1 ng/mL) concentrations (left), which can also be visualized by similar EdU staining for both cultures (right). Error bars are standard deviation and n=3.

FIG. 17 depicts characterization of Oligo Labeling Reaction of Niche Ligands, Fibroblast Growth Factor-2 (FGF-2) and EphB4-Binding Peptide, using Click Chemistry. (A) The strategy for labeling the niche ligand, FGF-2, is identical to that of labeling the fluorescent proteins; however, in order to conjugate an oligo to the short EphB4 peptide, TNYLFSPNGPIARAW (SEQ ID NO:16), a slight variation in the reaction stoichiometries was made to accommodate the small peptide size (˜2 kDa), which made it difficult to remove excess DBCO crosslinker via a spin column. (B) SDS-PAGE was used to assess FGF-2 reaction efficiency (left), and a 20% PA gel was employed to analyze the peptide reaction (right). For both scenarios, no unlabeled oligo was detected, suggesting that all of the fluorescent oligo label was reacted to the niche ligand of interest.

FIG. 18 depicts multicomponent DNA Patterns Enable Controlled, High-Throughput Studies of Adult Neural Stem Cell (NSC) Niche Solid-Phase Ligand Cues, Fibroblast Growth Factor-2 (FGF-2) and Ephrin-B2, at the Single-Cell Level. (A) Adult NSCs reside within a dynamic niche where they receive and integrate a wide variety of extrinsic cues that ultimately instruct NSC fate decisions. FGF-2 and ephrin-B2 are two niche cues that promote opposing cell fates, where FGF-2 induces self-renewal, and ephrin-B2 promotes neuronal differentiation (top). The spatial presentation of (a) FGF-2 and (b) an ephrin-B2-mimetic peptide, TNYLFSPNGPIARAWC (SEQ ID NO:17), can be controlled via DNA-directed assembly. Tunable solid-phase presentation can be achieved by modulating the concentration of surface-patterned DNA as visualized by the representative microislands with increasing fluorescent intensities. (B) Single NSC microislands were assembled containing a range of patterned ligand concentrations of either FGF-2 or ephrin-B2 peptide (a) Different concentrations of patterned FGF-2 resulted in similar proliferation and differentiation profiles, even at low concentrations. (b) For the ephrin-B2 peptide, however, a minimum ligand concentration was necessary to drive high neuronal differentiation (right). Error bars are standard deviation and n values can be found in Table S3. All scale bars represent 100 μm.

FIG. 19 depicts Cell Body Segmentation using ilastik Software. One of the key challenges faced when analyzing the time-lapse experiments was processing the large number of microisland samples and their even larger associated time-lapse data sets. To address this challenge, a custom computational analysis pipeline was developed to detect and quantify cells within each protein region at each timepoint. In short, ilastik software (66) was employed to enable high-throughput segmentation and identification of cell bodies. (A) A pipeline was established based on user training of the software to distinguish between cell bodies (green) and background (red). Specifically, a handful of timepoints were analyzed manually, which included more challenging case scenarios in which cells were closely associated, spread out, more transparent, etc. Three example time-lapse frames (top) and their associated user-defined training segmentation (bottom) are displayed. (B) The established pipeline was then applied via batch processing to every frame of a 4-day time-lapse video. Representative images of bright field images (top) and their segmentation results (bottom) are provided.

FIG. 20 shows an overview of Custom Fiji Script for Counting Cells. The segmented time-lapse videos achieved using ilastik were then imported and analyzed via a custom macro script in Fiji. (A) The first step included converting the segmentation images into binary images and subsequently conducting post processing by filling holes and watershedding. Example timepoints from Days 0, 1, 2, 3, and 4 are provided. (B) The second step involved generating regions of interest (ROIs) based upon the fluorescent signal that is coupled to the protein patterns. (C) Finally, particle analysis was then conducted for three scenarios: 1) total cell body counts within the entire frame of view, 2) cell body counts within ROI 1, and 3) cell body counts within ROI 2. Based on these counts, the number of cell bodies within each of the protein regions as well as those that span both regions could be extrapolated for each timepoint.

FIG. 21 depicts tracking changes in Average Cell Occupancy within FGF-2 over Time for Each Individual Microisland. (A) Microislands were binned into one of three categories based on Day 5 immunostaining results: “Low (0%)”, “Medium (0-100%)”, and “High (100%)” Tuj1+ neuronal differentiation. For each microisland, average cell occupancy within FGF-2 was then monitored for each day of the 4-day time-lapse. The microisland's trajectory is depicted as a line connecting each of these 4 points. (B) Time-lapse snapshots (left) of a microisland exhibiting 100% neuronal differentiation (right) despite having near 100% FGF-2 occupancy throughout the 4-day culture reveals dynamic neurite processes occupying both FGF-2 and ephrin-B2 patterns.

FIG. 22 shows polydimethylsiloxane (PDMS) Stamping Protocol to Enable Cell Patterning. PDMS flow cells are affixed within each well of a 4-well chamber slide to enable cycling of an oligo-labeled cell suspension across the DNA-patterned surface. The PDMS flow cell concentrates the cells to the surface as well as increases the probability of hybridization between the complementary cell-bearing and surface-patterned oligos. The latter of which is achieved by the repeated action of removing 5 μL of the cell suspension from the outlet and re-injecting into the inlet, thereby slowly cycling the cells across the surface until cells are patterned efficiently. In order to secure PDMS flow cells to the glass slide, a PDMS stamping protocol was employed. (A) Degassed PDMS was spun onto a plain glass slide at 4,000 RPM for 30 seconds to generate a thin film of PDMS. The flow cell was then stamped against this surface, coating only the pillars of the flow cell. (B) The flow cell is then positioned carefully over the DNA-patterned wells to ensure that, not only are the DNA patterns contained within the flow cell chamber, but also the flow cell must fit within the 4-well gasket chamber. The glass slide assembly is then heated at 65° C. for 1 hour to cure the PDMS “glue”, creating a bond strong enough to secure the flow cell throughout both the cell patterning steps as well as the 5-day differentiation experiment. (C) (a) A representative image of PDMS flow cells positioned within each well and (b) demonstration of capillary action drawing food coloring through the flow cell without the need of plasma treatment due to the tall flow channel height of 150 μm.

FIG. 23 depicts DNA-Directed Cell Patterning using Photolithographically-Defined Surface DNA Patterns. (A) Microfabricated patterns of 20-bp single-stranded oligonucleotides direct the assembly of cell types of interest with high spatial precision via hybridization between complementary surface-conjugated and cell-labeling oligo strands. Cells are labeled with DNA through two successive incubation steps with lipid-modified oligo strands. While the first DNA imparts specificity, as the sequence of this strand is complementary to that of the DNA patterned onto the substrate, the second lipid-modified “co-anchor” strand tethers the first strand into the cell membrane. To achieve high patterning efficiencies, DNA-labeled cells are flooded first, via a PDMS flow cell, onto the surface of the DNA-patterned substrate. The cell suspension is then cycled slowly across the surface via the repeated action of pipetting cells into the flow cell inlet, retrieving the cells from the outlet, and re-pipetting into the inlet. Excess cells are washed off by flooding the flow cell with PBS multiple times, revealing cell patterns. (B) The resulting bulk adult neural stem cell patterns can be discerned by eye when DNA patterns are on the order of 100's of microns, as is the case for the displayed “Berkeley” patterns.

FIG. 24 shows an overview of Surface-Patterned DNA Sequences and their Complementary Fluorescent, Cell-Labeling, and Ligand-Labeling Oligonucleotides.

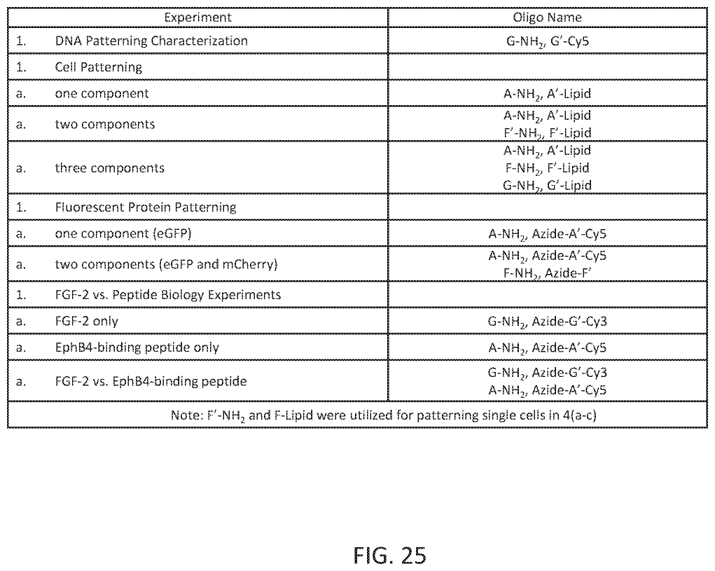

FIG. 25 shows an overview of Complementary Pairs of DNA Strands used for Characterization, Cell Patterning, Ligand Patterning, and Biological Experiments.

FIG. 26 shows an in-Depth Report of Experimental Sample Number “n”.

FIG. 27 shows a DNase-based strategy to achieve rapid, one-pot cleavage. (A) Temporal control over DNA-assembled ligands was achieved through incubation with DNase. (i) A 20 base-pair, single-stranded oligonucleotide was conjugated with high spatial control onto an aldehyde-functionalized glass slide using photolithographic techniques. (ii) Surface-tethered oligonucleotides instructed ligand assembly via hybridization with complementary oligonucleotides labeling a ligand of interest. (iii) The addition of DNase to DNA-assembled ligands induced hydrolysis of phosphodiester bonds, resulting in cleavage and subsequent release of the patterned ligand. (B) (i) Release kinetics of a hybridized fluorescent oligonucleotide were tested at different timepoints (1 min, 5 min, and 15 min) with different enzyme units (1 U, 10 U, and 100 U) of DNase prepared in NSC media (n=3; error bars represent standard deviation). (ii) Representative images of fluorescent DNA patterns 15 minutes post-incubation with different enzyme units of DNase. Scale bar represents 100 μm.

FIG. 28 shows DNase-based cleavage of patterned fibroblast growth factor-2 (FGF-2) induced changes in single NSC proliferation. (A) A fluorescent oligonucleotide was conjugated to FGF-2 using click chemistry, and the conjugate was patterned within a large array of microisland features containing single NSCs using DNA-based assembly. FGF-2 patterns were visualized by imaging the fluorescent tag. Zoomed-in, overlay image shows representative 3×3 array of single-cell microislands patterned within FGF-2 (right). (B) (i) FGF-2 was patterned and cleaved with DNase at controlled timepoints (Day 0 and Day 2) during a 4-day culture, and proliferation rate was compared between conditions. (ii) Representative images of transmitted light (TL) and DAPI-stained microislands on Day 4 highlight different proliferation rates for various cleavage conditions. Scale bar represents 100 μm.

FIG. 29 shows Encoding DNA cleavage specificity with restriction sites. (A) The incorporation of restriction sites into patterned DNA strands enabled targeted, site-specific cleavage upon incubation with a restriction enzyme. (B) Cleavage kinetics of different enzyme units (10 U, 100 U, and 1000 U) of (i) Eco RI and (ii) Bam HI prepared in different media conditions (N2+0.1 mg/mL BSA, 1× CutSmart in N2, and 1× CutSmart in water) were tested when incubated with hybridized fluorescent oligos containing either Eco RI or Bam HI cut sites for 1 hour at 37° C. (n=3; error bars represent standard deviation). (C) The combined use of unique restriction sites and DNase enabled temporal control over multiple DNA-assembled signals as shown by the sequential cleavage of a (i) Cy5-hybridized oligonucleotide possessing an Eco RI cut site, (ii) Cy3-hybridized oligonucleotide possessing a Bam HI cut site, and (iii) AlexaFluor488-hybridized oligonucleotide with the use of DNase. The first cleaved DNA strand was designed to retain functionality post-cleavage, enabling the re-hybridization of a new Cy3-fluorescent oligo. Scale bar represents 100 μm.

FIG. 30 depicts RNA-guided, site-specific cleavage with Streptococcus pyogenes Cas9 (spCas9) ribonucleoprotein (RNP). (A) spCas9 RNP is assembled with a 20-base pair (bp) gRNA targeting a surface-patterned DNA oligo that has been hybridized with a ligand of interest (left). The target sequence is immediately adjacent to the spCas9 PAM sequence, NGG, and contains 15-bp flanking sequences (right). For characterization experiments, a complementary DNA strand with a Cy5 modification at the 3′ end was employed for visualization and quantification. (B) Cas9 RNP cleavage kinetics of 50 nmole was tested for different incubation times (1, 5, and 15 min) in RNP buffer at 37° C. n=3 and error bars represent standard deviation. (C) Initial studies (i.e. one replicate) suggest that titratable cleavage can be achieved through multiple 15-min RNP doses. Scale bar represents 100 μm.

FIG. 31 depicts DNA-Patterned, High-Throughput Single Neural Stem Cell (NSC) Array. Surface DNA patterns instruct high-throughput arrays of single adult neural stem cells (NSCs) via hybridization between surface-tethered and cell-bearing complementary oligonucleotides. White arrows highlight single NSC capture, red arrows highlight no capture, and yellow arrows highlight floating cells not in-plane.

FIG. 32 depicts Patterned Single Neural Stem Cell (NSC) Retention Following Laminin Incubation and DNase Treatment. A one-hour incubation step with 10 μg/mL of laminin was conducted prior to DNase treatment to allow cells to attach to the laminin-coated glass substrate and no longer rely on its surface-tethered oligonucleotides to position it onto the surface. An average single-cell pattern retention rate of 75-80% was observed. Of the cells that were removed, the majority were located at the inlet of the polydimethoxysilane flow cell where they experienced the most shear. Red arrows highlight cells that were removed during the DNase treatment.

FIG. 33 shows the effects of DNase treatment on adult neural stem cell (NSC) differentiation. NSCs were treated for 30-minutes at 37° C. with different concentrations of DNase prepared in NSC media (i.e. N2 media) prior to inducing mixed differentiation for 5 days. Immunostaining analysis of neuronal and astrocytic fate commitment revealed similar differentiation proportions for each condition. n=4 and error bars are standard deviation.

FIG. 34 depicts Live/Dead Assessment of Adult Neural Stem Cells (NSCs) Following 1× CutSmart Treatment in N2 Media for Various Incubation Times. Live/dead assessment of adult neural stem cells (NSCs) following 1× CutSmart treatment in N2 media for various incubation times.

FIG. 35 shows the effects of 1× CutSmart buffer on NSC behavior. Effects of 1× CutSmart buffer prepared in N2 Media on NSC differentiation and proliferation. n=4 and error bars are standard deviation.

FIG. 36 depicts Cleavage Kinetics of Plasmid in Different Buffer Conditions using Gel Format. (a) A ˜11.6 kbp plasmid containing two (i) Bam HI and (ii) Eco RI cut sites was employed to screen restriction enzyme cleavage kinetics. (b) Restriction enzymes, (i) Bam HI and (ii) Eco RI, were incubated for different incubation times (5 min, 30 min, 60 min) in various buffer conditions (1× CutSmart in water, 1× CutSmart in N2 media, and N2 media+0.1 mg/mL BSA) prior to running out the cleavage products on a 1% agarose TAE gel and imaged with SyberSafe.

FIG. 37 depicts specificity of Restriction Enzyme-Based Cleavage. (a) To demonstrate the specificity of restriction enzyme-based cleavage, an incorrect Bam HI oligo was patterned onto an aldehyde glass substrate using photolithography. (b) Different concentrations of Bam HI prepared in 1× CutSmart buffer were incubated for one hour following the hybridization of a fluorescent complementary oligo, revealing little to no cleavage between conditions. n=3 and error bars are standard deviation.

FIG. 38 depicts in-Depth Visualization of Rehybridization Strategy Post Restriction Enzyme-Based Cleavage. (i) Eco RI oligo is conjugated to a glass substrate by reacting the terminal amine modification on the oligo to the aldehyde group functionalized to the glass substrate. (ii) A 20-bp complementary oligo containing an Eco RI cut site (GAATTC) and Cy5 fluorescent modification is hybridized to the surface-tethered oligo. (iii) The addition of Eco RI restriction enzyme releases the hybridized fluorescent DNA portion. (iv) The unhybridized surface DNA can then hybridize a new fluorescent oligo strand.

FIG. 39 depicts SpCas9 RNP Cleavage Kinetics Screen in Gel Format. SpCas9 RNP cleavage kinetics were tested on the same surface-patterned and complementary fluorescent DNA strands utilized for DNA patterning. Both a 10-fold molar excess of SpCas RNP (right) and 100-fold molar excess (right) were investigated as well as specificity of cleavage through the incorporation of a non-targeting single-guide RNA (sgRNA).

FIG. 40 depicts an overview of DNA Sequences.

### Example 2

Here, a DNA-based solution to achieve temporal control over solid-phase ligand presentation is described, where the DNA-based solution enables investigations of how temporal patterns of extrinsic cues converge into cell fate decisions. Within the last decade, DNA has transcended its traditional role of storing inheritable information and orchestrating protein production by functioning as an engineering building block due to its ease in programmability, precise nanoscale geometry, and robust hybridization. Inspired by DNA's unique properties, the use of photolithography has been leveraged to fabricate multiplexed DNA substrates that assemble complex biological signaling environments in vitro in a controlled, bottom-up manner. Patterned 20-base pair (bp) oligonucleotides direct the capture of heterogenous cell types as well as solid-phase ligands by hybridizing with their complementary DNA strands, each conjugated with a signaling component (e.g. cell or solid-phase ligand) of interest. To impart temporal control over patterned signaling components within the platform, inspiration from biology continues to be drawn by utilizing enzymes that recognize specific DNA sequences. Various nuclease-based strategies can be incorporated to achieve rapid cleavage of a patterned single ligand or multiplexed cleavage over multiple signaling ligands of interest. Furthermore, the combination of cleavage and hybridization provides the necessary foundation for recapitulating complex temporal logic.

FIG. 27 a illustrates the first proposed strategy of utilizing deoxyribonuclease (DNase) to engineer control over the timing and duration of a presented signal. Firstly, photolithography is employed to pattern photoresist, guiding the covalent conjugation of a 20-bp oligonucleotide with micron-scale spatial resolution. The functional, patterned DNA strand then assembles a ligand-of-interest upon hybridization with its complementary oligonucleotide, which has been tethered to the ligand. The controlled introduction of DNase, which acts by cleaving the phosphodiester linkages of the DNA backbone, releases the DNA-hybridized ligands and is followed by a wash step to remove the cleaved products. Due to this enzyme's lack of specificity, this particular approach enables a complete removal of all presented ligands, regardless of patterned DNA sequence. To enable visualization and quantification of DNase-based cleavage kinetics, surface DNA patterns were hybridized to a fluorescent complementary oligonucleotide. We, then, introduced different concentrations of DNase (1 U, 10 U, and 100 U) and analyzed residual fluorescence after various incubation times (1 min, 5 min, and 15 min). As expected, tunable cleavage kinetics was observed by either increasing incubation time or increasing the concentration of DNase (FIG. 27 b). Particularly, at 100 units of DNase, rapid cleavage of all patterned DNA within minutes was achieved. Thus, this strategy provides a simple and robust method to achieved rapid on-off kinetics for solid-phase ligands.

To provide a proof-of-concept demonstration that DNase-based cleavage can be applied within a biological system and induce changes in cell behavior, the DNA-patterning platform was employed to address single NSCs (FIG. 31) within square microisland features containing patterned fibroblast growth factor-2 (FGF-2), a key signaling ligand that promotes proliferation. As illustrated in FIG. 28 a, a fluorescent tag was included at the 3′ end of the oligonucleotide labeling FGF-2 to serve as a readout of protein localization, and the use of photolithography generated a large array of 100's of single-cell/FGF-2 microisland cultures. To achieve temporal control over FGF-2 presentation, DNase was introduced at different timepoints post-cell patterning to vary the persistence of ligand exposure and its effects on single NSC proliferation rate were subsequently assessed. To avoid removing DNA-patterned NSCs upon the introduction of DNase, cells were incubated with laminin for one hour to allow for cells to adhere to the laminin-coated microislands and no longer rely on the DNA-tether (FIG. 32). The removal of FGF-2 at different time points throughout the 4-day culture resulted in a spectrum of proliferation rates (FIG. 28 b(i)) that increased from NSCs that lacked patterned FGF-2 to those that had immediate cleavage on Day 0, and cleavage on Day 2. These differences in proliferation rate can also be visualized through DAPI staining (FIG. 28 b(ii)). Through additional experiments, it was shown that DNase has low cytotoxicity and minimal effect on NSC differentiation (FIGS. 33 and 34).

Though the application of DNase achieves temporal control over single DNA-presented ligands, biological systems are often comprised of multiple cues cooperating synergistically or antagonistically. Multicomponent control is required to resolve the temporal parameters governing these complex interactions. The use of orthogonal surface oligonucleotide strands programmed with unique sequences provides a solution for coordinating the DNA-based assembly of multiple signaling ligands (citation). However, in order to obtain cleavage specificity, programming targeted cleavage sites into the patterned oligonucleotides is proposed. As demonstrated in FIG. 29 a, the incorporation of restriction sites and the subsequent incubation of the corresponding restriction enzyme. To assess the feasibility of this approach, two DNA strands were designed and tested: one encoded with a Bam HI restriction site (GGATCC) and another with an Eco RI restriction site (GAATTC). Complementary fluorescent oligonucleotides were then hybridized and cleavage following incubation with different concentrations of the high-fidelity restriction enzymes was quantified. As highlighted in FIG. 29 b(ii), one of the key determinants of cleavage efficiency was the incubation buffer used during cleavage. A comparison of 1× CutSmart buffer, which is commonly employed for molecular cloning, against 1×PBS and NSC media (i.e. N2 media) revealed that CutSmart buffer was far more efficient for both enzymes. However, in the case of high Bam HI concentration at 1000 U, this discrepancy was negligible, as nearly complete cleavage was achieved for all three buffers after a one-hour incubation. Given the discrepancy in cutting kinetics for the different buffers, whether cells could tolerate CutSmart buffer was investigated. Therefore, the effects of incubating 1× CutSmart buffer prepared in N2 media with adult NSCs were tested. For a one-hour incubation, cell viability remained high (FIG. 34), and little to no effect on NSC proliferation and differentiation was identified (FIG. 35).

Through characterization experiments, variability in restriction enzyme cleavage activity was also discovered. Specifically, Bam HI demonstrated faster cutting kinetics while also demonstrating more tolerance to buffer composition. Eco RI exhibited far poorer cutting in N2 media across the different concentrations—a result most likely attributed to sub-optimal concentration of magnesium, which serves as a necessary co-factor for restriction enzyme activity, and/or to the presence of high salt concentration. As shown in FIG. 36, these results were recapitulated in a gel format upon repeating the buffer and concentration conditions using a plasmid as a substrate, one that contained two Bam HI and two Eco RI cut sites. Therefore, when selecting appropriate restriction enzymes and buffer conditions, a rapid gel cleavage screen can be conducted to inform optimal oligonucleotide sequence design and ensure appropriate cleavage conditions. The robust specificity of this restriction enzyme-based cleavage strategy was highlighted further by testing the addition of Bam HI enzyme to a surface-patterned DNA strand containing a restriction site that closely resembles the correct Bam HI sequence yet deviates by one base pair (FIG. 37). No significant cutting was detected across different Bam HI concentrations.

Thus, programming restriction sites into patterned DNA strands is one strategy for imparting the necessary specificity to achieve multiplexed temporal control. Given that the discovery of CRISPR-Cas9 has revolutionized gene-editing therapeutics by enabling targeted Cas9-mediated gene correction, whether Streptococcus pyogenes (SpCas9) ribonucleoproteins (RNPs) could be applied to the DNA patterning system in a parallel approach was investigated. Analogous to type II restriction endonucleases, which assemble as homodimers that each recognize and cleave within symmetric recognition sequences, SpCas9 contains two nuclease domains that together generate double-stranded breaks. Thus, when implemented into the platform, Cas9-mediated cleavage releases the DNA-presented ligand, which can then be washed away.

As illustrated in FIG. 29 a, a single-stranded oligonucleotide amenable to SpCas9cleavage was designed and patterned as it contains a 20-bp target sequence located directly adjacent to the SpCas9 protospacer adjacent motif (PAM). 15-bp flanking regions were included to accommodate the bulky ˜160 kDa Cas9 protein, providing both accessibility to the surface-tethered oligo and adequate length for R-loop formation by base-pair hybridization between the gRNA and target DNA sequence. To validate that the assembled Cas9 RNP could target and cleave the designed oligos, cleavage efficiencies were first screened in a gel format similar to the previously described restriction enzyme strategy in the present disclosure, identifying rapid cleavage for both 10-fold and 100-fold excess of RNP (FIG. 38). The CRISPR oligo was then patterned using photolithography. Upon hybridizing the fluorescent complementary oligo, the kinetics of SpCas9 RNP-mediated surface DNA cleavage were investigated, and incubation times that ranged from 1 min to 15 minutes were tested (FIG. 29 b). A cutting saturation effect—which most likely can be attributed to a lack of SpCas9 RNP displacement post-cleavage—was discovered. It was also found that this saturation was reached within minutes, which highlights the rapid yet titratable action of SpCas9 RNP. This is in contrast to the previous restriction enzyme strategy that demonstrated increased cutting of DNA-presented ligand upon increased incubation times. To achieve various degrees of cutting, multiple doses at a fixed RNP concentration were tested. As shown in FIG. 29 c, repeated application of Cas9 RNP (1×, 2×, and 3×) resulted in increased cleavage. Interestingly, preliminary experiments revealed that increasing the RNP concentration does not result in increased cleavage efficiencies. To provide the initial proof-of-concept of the targeted specificity of Cas9 RNP within the system, two-component DNA patterns, in which one oligo strand that contains the target sequence complementary to the gRNA is patterned within the same square microisland area with a second strand that does not, were fabricated. As expected, upon the addition of multiple Cas9 doses, cleavage was achieved for one component and not the other.

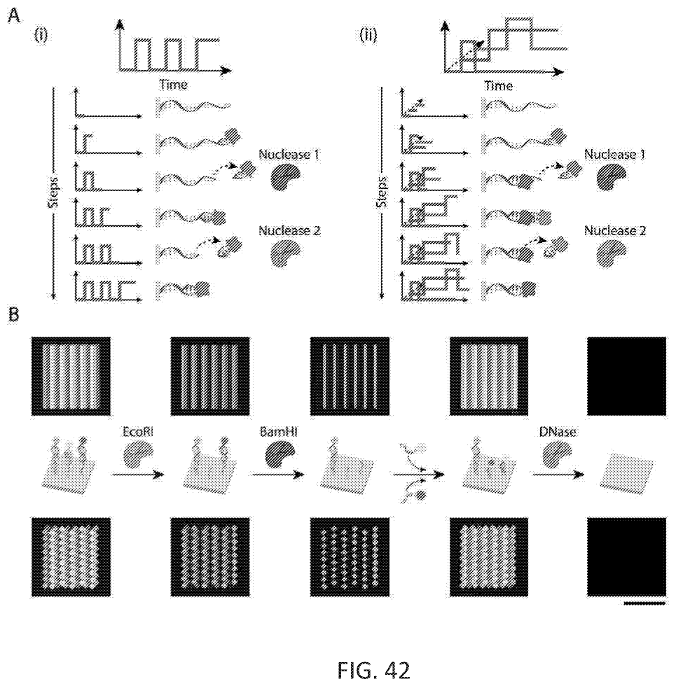

The application of DNase, restriction enzymes, and Cas9 RNPs within the lithographic DNA-patterning platform provides not only a systematic and controlled approach to model temporal solid-phase ligand signaling but also a foundation for engineering more complex temporal logic, capturing various modes of ligand presentation. As illustrated in FIG. 42 a, one approach is to extend the length of the surface-tethered oligonucleotide and program it to contain multiple cleavage sequences. In doing so, cycled on-off kinetics can be obtained for a single ligand through repeated hybridization and cleavage (FIG. 42 a(i)). Additionally, heterogeneous cues can be presented and displaced in parallel on the same strand with the added advantage of nanoscale spatial control between ligands through their strategic hybridization given DNA's predictable structure (FIG. 42 a(ii)). A second approach involves spatiotemporal control over micropatterned regions, which is demonstrated in FIG. 42 b using hybridized fluorescent oligonucleotide strands. Three unique fluorophores were patterned initially, and the subsequent introduction of Eco RI and Bam HI removed the fluorescent oligos depicted by the yellow and purple patterns, respectively. Both cleavages exposed residual, functional oligonucleotide strands that were then hybridized to new complementary strands that inverted the initial fluorophore presentation. Finally, the introduction of DNase removed all DNA-presented fluorophores. In-depth illustration of the DNA sequences employed for this cleavage and re-hybridization approach can be visualized in FIG. 39.

Here, DNA-based strategies for engineering temporal control over presented solid-phase ligands—strategies that enable recapitulating and dissecting how temporal parameters modulate ligand action—are described. The platform harnesses photolithography to fabricate surface DNA patterns comprised of multiplexed oligonucleotide strands which, in turn, assemble heterogeneous ligands through the hybridization between surface-tethered oligonucleotides and ligands labelled with the unique, complementary strands. While it was previously demonstrated that employing photolithography imparts tight spatial control over ligand presentation, integrating temporal control into the system by capitalizing on biology's natural toolbox of nucleases to cleave DNA-presented ligands was proposed. One of the key advantages of adopting nucleases is their biocompatibility, which is in contrast to previous strategies that apply UV light, induce pH changes, or tune temperature to achieve temporal modulations—thus, inducing cytotoxicity and/or altering normal cell behavior. A second key advantage of nuclease-mediated temporal control is the ease of method adoption as both custom oligonucleotides as well as various nucleases are widely accessible. Finally, the third advantage is the cleavage tunability by selecting different classes of nucleases. With DNase, nonspecific yet rapid cleavage of all hybridized ligands, which is particularly well suited for controlling solid-phase ligand persistence of a single ligand and investigating the effects of timing and strength/duration on cell behavior, was demonstrated. For signaling environments in which multiple signals are operating together, both restriction endonucleases and SpCas9 RNP's can be utilized to achieve cleavage specificity or multiplexed temporal control. Finally, even more sophisticated strategies can be derived from these capabilities, modeling more complex networks. Altogether, these nuclease-mediated strategies can easily be integrated into a diverse span of biological systems, providing a facile and robust approach to dissect the effects of temporal signaling.

### Methods

### Surface DNA Patterning Using Photolithography

20 base-pair, single-stranded oligonucleotides (IDT) were patterned onto an aldehyde glass slide (Schott Nexterion). Briefly, microfabricated positive photoresist (Shipley S1813) served as a physical mask, selectively exposing aldehyde surface regions and subsequently guiding the conjugation of amine-terminated oligonucleotides with high spatial control. An oligonucleotide solution prepared in 50 mM sodium phosphate buffer (pH=8.5) was dropcast over the patterned photoresist, heated to induce condensation, and treated with 0.05% sodium borohydride in 1×PBS for 10 minutes to conjugate covalently the amine-terminated oligonucleotide to the aldehyde substrate. Photoresist was removed by rinsing slides with acetone, followed by de-ionized (DI) water, and dried under a dry nitrogen stream. Slides were stored under vacuum until ready to use. DNA sequences are provided in FIG. 40.

To visualize patterns, Millicell EZ 4-well chambers (Millipore) were first secured onto the glass slide, and the surface was blocked for 1 hour on a shaker by adding 1 mL of blocking buffer (2% bovine serum albumin (BSA) in 1×PBS) to each well. The 2% BSA was exchanged using a pipette with 250 μL/well of a 0.2 μM solution of complementary oligonucleotide containing a fluorescent tag prepared in the same blocking buffer. Following a 5-minute incubation on a shaker to allow for hybridization, wells were rinsed 4× with 1×PBS to remove unbound fluorescent oligonucleotides and stored in fresh 1×PBS during imaging.

### Cell Culture

Adult rat hippocampal neural stem cells (NSCs) were isolated previously from 6-week-old female Fischer 344 rats. To promote monolayer adhesion, NSCs were cultured on polystyrene plates coated with 10 μg/mL poly-L-ornithine hydrobromide (Sigma) in sterile DI water overnight at room temperature and 5 μg/mL of laminin (Invitrogen) in sterile PBS overnight at 37° C. NSCs were maintained in Dulbecco's Modified Eagle Medium/Nutrient Mix F-12 (DMEM/F-12, Invitrogen) with 1% (v/v) N-2 Supplement (Invitrogen) and 20 ng/mL of basic fibroblast growth factor (FGF-2, Peprotech) and incubated at 37° C. and 5% CO₂. NSCs were passaged upon 80% confluency using Accutase (Innovative Cell Technologies). For differentiation studies, NSCs were cultured in normal culture media supplemented with 1% fetal bovine serum (Invitrogen), 1 μM retinoic acid (Enzo Life Sciences), and 1% penicillin-streptomycin (Gibco) in DMEM/F-12+N-2 Supplement. For studies involving protein patterns, NSCs were cultured in maintenance media (DMEM/F-12+N-2) supplemented with 0.1 ng/mL of FGF-2 to promote low proliferation and differentiation.

### DNA-Based Single NSC Patterning

High-throughput capture of thousands of single NSCs is achieved by first labeling cell membranes with lipid-conjugated oligonucleotides containing the complementary sequence to surface-patterned, 15 μm-diameter DNA spot features. The prepared DNA-tethered cell suspension is then flowed 10-20 times across the DNA substrate with the aid of a polydimethoxysilane (PDMS) flow chamber to promote hybridization between surface-bearing and cell-labeling oligos—thus, achieving cell capture. Both the cell-labeling process and flow steps are described previously in detail.

### DNase-Based Cleavage Experiments

Deoxyribonuclease I (DNase) (Worthington, LS002138) was supplied by the manufacturer as a lyophilized power and was re-suspended in NSC media (DMEM/F-12+1% N-2 Supplement) at 1,000 Kunitz units/mL. For DNase-cleavage characterization experiments, stock DNase was diluted further to the appropriate Kunitz units in 250 μL of media and subsequently incubated at 37° C. within each DNA-patterned well for the designated amount of time. Wells were then rinsed 4× with 1×PBS to remove cleaved DNA.

For DNase-cleavage experiments of DNA-hybridized fibroblast-growth factor 2 (FGF-2), patterned single NSCs were first incubated with 20 μg/mL of laminin for 1 hour at 37° C. to ensure that cells no longer rely on DNA to be tethered to the surface and, thus, not removed upon the introduction of DNase. 1000 units of DNase was then introduced to cells by pipetting into the PDMS flow cell and allowed to incubate for 15 minutes at 37° C. Wash steps were conducted by cycling PBS into and out of the flow cell 10×. A total of three DNase treatment was conducted to ensure complete cleavage of patterned FGF-2 and validated upon imaging as an observed loss of Cy3 signal.

### Restriction Enzyme-Based Cleavage Experiments

For DNA-patterning experiments, high-fidelity Bam HI and Eco RI (NEB) were prepared in the following buffers: 1× CutSmart (NEB) diluted in water, 1× CutSmart diluted in NSC media (DMEM/F-12+1% N-2 Supplement), and/or NSC media supplemented with 0.1 mg/mL bovine-serum albumin (BSA). 250 μL of prepared restriction enzyme solution containing the desired units and buffer were then added to fluorescently-hybridized DNA-patterned slides, prepared as described above.

For restriction enzyme digest characterization experiments, 20 units of high-fidelity Bam HI and Eco RI were prepared in the aforementioned buffers and incubated with 500 ng of a 11.6 kb plasmid containing two Eco RI cut sites and two Bam HI cut sites in a total volume of 50 μL for 5 min, 30 min, or 60 min at 37° C. Digest efficiency was assessed by running products on a 1% Tris-Acetate-EDTA (TAE) agarose gel, and DNA bands were visualized using SyberSafe (Thermo Fisher Scientific).

Streptococcus pyogenes Cas9 (SpCas9) Ribonucleoprotein (RNP)-Based Cleavage Experiments

To assemble the RNP, recombinant SpCas9 was diluted to 10 μM in RNP buffer (20 mM Tris-HCl, 100 mM KCl, 5% (v/v) glycerol, 5 mM MgCl₂, 1 mM TCEP, 50 μg/mL, pH=7.5) and added to 12 μM of folded single guide RNA (sgRNA) prepared in folding buffer (20 mM HEPES, 150 mM NaCl, pH=7.5), which was transcribed and purified in the Doudna Lab as described previously. The two components were incubated at 37° C. for 10 minutes and subsequently diluted further in RNP buffer at the desired concentration prior to being added to DNA-patterned substrate.

### Statistical Analysis

All statistical analysis was performed in MATLAB (R2018a). One-way Analysis of Variance (ANOVA) was used to test for significant differences between variable means. A p-value of less than 0.05 for ANOVA was considered significant. For data with a significant ANOVA result, the Tukey-Kramer method was used to compare between individual groups and test for significance. A p-value of less than 0.05 for the Tukey-Kramer method was considered significant. Details on replicates, ANOVA results, and Tukey-Kramer comparisons are provided in the figure captions.

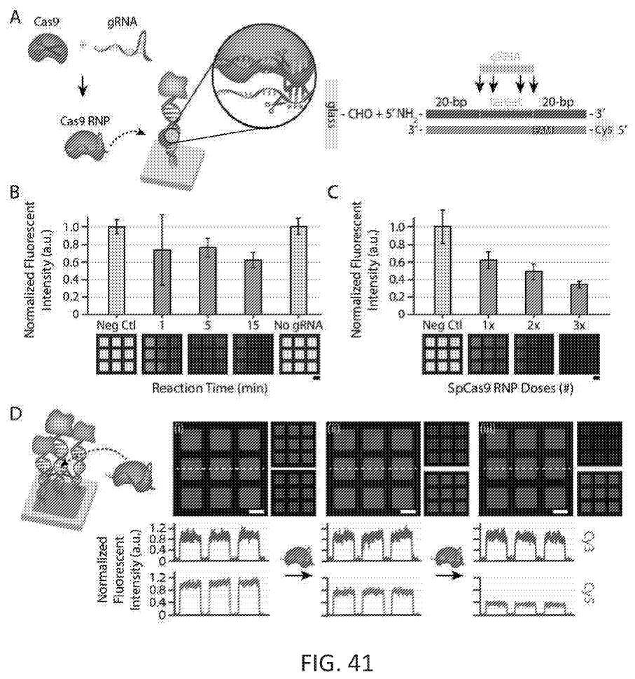

FIG. 41 depicts RNA-guided, site-specific cleavage with Streptococcus pyogenes Cas9 (spCas9) ribonucleoprotein (RNP). (A) spCas9 RNP is assembled with a 20-base pair (bp) gRNA targeting a surface-patterned DNA oligo that has been hybridized with a ligand of interest (left). The target sequence is immediately adjacent to the spCas9 PAM sequence, NGG, and contains 15-bp flanking sequences (right). For characterization experiments, a complementary DNA strand with a Cy5 modification at the 3′ end was employed for visualization and quantification. (B) Cas9 RNP cleavage kinetics of 50 nmole was tested for different incubation times (1, 5, and 15 min) in RNP buffer at 37° C. n=3 and error bars represent standard deviation. (C) Titratable cleavage can be achieved through multiple 15-min RNP doses. Error bars are standard deviation and n=3. Scale bars represent 100 μm.

FIG. 42 depicts engineering complex temporal signaling logic. The combined use of programmed hybridization and cleavage sets the stage for more complex temporal strategies. This can be achieved within (a) a single patterned oligonucleotide strand in which (i) cycled on-off kinetics can be achieved for a single ligand or (ii) parallel, heterogeneous temporal control can be obtained for multiple ligands. (b) Spatiotemporal control can also be obtained over micropatterned regions as demonstrated by the sequential cleavage of Cy5-hybridized oligonucleotides possessing Eco RI cut sites (i.e. yellow pattern) and Cy3-hybridized oligonucleotides possessing Bam HI cut sites (purple pattern). Both cleaved DNA strands were designed to retain functionality post-cleavage, enabling the re-hybridization of new fluorescent oligo that inverted the original fluorophore patterns (i.e. yellow-purple to purple-yellow). Finally, the application of DNase removed all DNA patterns. Scale bar represents 100 μm.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising:
a) a solid support;
b) a plurality of tethered oligonucleotides, wherein the tethered oligonucleotides are attached to the solid support via the 5′ termini of the oligonucleotides in a patterned array, wherein each of the plurality of tethered oligonucleotides comprises a nucleotide sequence that, when hybridized to a complementary nucleotide sequence present in an untethered oligonucleotide, generates an enzyme cleavage site; and
c) a plurality of untethered oligonucleotides that are hybridized to the plurality of tethered oligonucleotides in the patterned array, wherein the untethered oligonucleotides each comprise:
i) the nucleotide sequence that generates an enzyme cleavage site, wherein the enzyme cleavage site is a restriction enzyme cleavage site or a site that is cleavable by a CRISPR/Cas effector polypeptide;
ii) a cell, or an effector molecule that affects an activity and/or a phenotype of a cell, wherein the cell or the effector molecule is attached to the untethered oligonucleotide at the 5′ end of the untethered oligonucleotides; and
iii) a fluorophore.

2. The composition of claim 1, wherein the effector molecule is a polypeptide.

3. The composition of claim 1, wherein the effector molecule comprises a lipid.

4. The composition of claim 1, wherein the effector molecule comprises an oligosaccharide.

5. The composition of any one of claims 1-4, wherein the enzyme cleavage site is:
a) a restriction enzyme cleavage site; or
b) a site that is cleavable by a CRISPR/Cas effector polypeptide when the CRISPR/Cas effector polypeptide is complexed with a guide RNA.

6. The composition of any one of claims 1-5, wherein the composition comprises at least a first, a second, and a third plurality of hybridized, untethered oligonucleotides that are bound to the tethered oligonucleotides in the patterned array, wherein:
a) the first plurality of hybridized, untethered oligonucleotides comprises, bound to the 5′ end of the oligonucleotides, a first effector molecule, wherein the first plurality of hybridized, untethered oligonucleotides generates a first enzyme cleavage site; and
b) the second plurality of hybridized, untethered oligonucleotides comprises, bound to the 5′ end of the oligonucleotides, a second effector molecule, wherein the second plurality of hybridized, untethered oligonucleotides generates a second enzyme cleavage site; and
c) the third plurality of hybridized, untethered oligonucleotides comprises, bound to the 5′ end of the oligonucleotides, a target cell.

7. The composition of claim 6, further comprising a fourth plurality of hybridized, untethered oligonucleotides, wherein the fourth plurality of hybridized, untethered oligonucleotides bound to the 5′ end of the oligonucleotides, a third effector molecule, wherein the fourth plurality of hybridized, untethered oligonucleotides generates a third enzyme cleavage site.

8. The composition of any one of claims 1-7, wherein the cell is a stem cell or a progenitor cell.

9. The composition of any one of claims 1-8, wherein the effector molecule is a growth factor, a hormone, an adhesion protein, a tumor-associated antigen, an integrin, a chemokine, a juxtacrine, an antibody, an extracellular matrix polypeptide, a co-stimulatory polypeptide, a T-cell receptor, a morphogen, a delta family protein, a Notch family protein, a Wnt polypeptide, or an Eph polypeptide.

10. A method of temporally modulating an activity and/or phenotype of a cell, the method comprising:
a) at a first time, contacting the composition of any one of claims 2-9 with a first enzyme that cleaves the first enzyme cleavage site, wherein said contacting results in removal of the first effector molecule from the target cell; and
b) determining the effect of the removal of the first effector molecule on an activity and/or phenotype of the cell.

11. The method of claim 10, comprising:
c) at a second time, contacting the composition of any one of claims 2-6 with a second enzyme that cleaves the second enzyme cleavage site, wherein said contacting results in removal of the second effector molecule from the target cell; and
d) determining the effect of the removal of the second effector molecule on an activity and/or phenotype of the cell.

12. A solid support comprising:
a) one or more patterns exposing an aldehyde-reactive substrate;
b) one or more tethered oligonucleotides covalently attached to the exposed aldehyde-reactive substrate within the one or more patterns via an amine-modified terminus at the 5′ end of the one or more tethered oligonucleotides,
wherein the one or more tethered oligonucleotides comprises a nucleotide sequence that, when hybridized to a complementary nucleotide sequence present in an untethered oligonucleotide, generates an enzyme cleavage site.

13. The solid support of claim 12, wherein the enzyme cleavage site is:
a) a restriction enzyme cleavage site; or
b) a site that is cleavable by a CRISPR/Cas effector polypeptide when the CRISPR/Cas effector polypeptide is complexed with a guide RNA.

14. The solid support of claim 12 or claim 13, wherein the one or more tethered oligonucleotides comprises a nucleotide sequence that hybridizes to a complementary nucleotide sequence present in an untethered oligonucleotide, generates an enzyme cleavage site, and wherein the untethered oligonucleotide comprises a target cell bound to the 5′ end of the untethered oligonucleotide.

15. The solid support of claim 12 or claim 13, wherein the one or more tethered oligonucleotides comprises a nucleotide sequence that hybridizes to a complementary nucleotide sequence present in an untethered oligonucleotide, generates an enzyme cleavage site, and wherein the untethered oligonucleotide comprises an effector molecule bound to the 5′ end of the untethered oligonucleotide.

16. The solid support of any one of claims 12-15, wherein the aldehyde-reactive substrate further comprises a grid that surrounds the one or more patterns.

17. The solid support of any one of claims 9-16, wherein the aldehyde-reactive substrate further comprises one or more alignment markers.

18. The solid support of any one of claims 9-17, wherein the one or more tethered oligonucleotides has a length of from 20 nucleotides to 50 nucleotides.

19. The solid support of any one of claims 9-18, wherein the one or more patterns exposing the aldehyde-reactive substrate comprises a diameter ranging from 50 nm-50 mm.

20. The solid support of any one of claims 9-19, wherein the one or more patterns exposing the aldehyde-reactive substrate comprises a diameter ranging from 2-5 μm, 5-10 μm, 10-15 μm, 15-20 μm, 20-25 μm, or 25-30 μm.

21. The solid support of any one of claims 9-20, wherein the one or more patterns comprises one or more micro-islands.

22. The solid support of any one of claims 9-21, wherein the grid is a polyacrylamide grid.

23. The solid support of any one of claims 9-22, further comprising one or more flow cells.

24. The solid support of claim 23, wherein the one or more flow cells is positioned over the plurality of tethered oligonucleotides.

25. A method of making a DNA patterned surface, the method comprising:
a) functionalizing a surface of a solid support with aldehyde groups to form an aldehyde-reactive substrate;
b) applying a cell-resistive layer onto the aldehyde-reactive substrate;
c) heating the cell-resistive layer;
d) applying a mask comprising one or more patterns to the cell-resistive layer;
e) exposing the aldehyde-reactive substrate, the cell-resistive layer, and the mask to create one or more patterns exposing the aldehyde-reactive substrate;
f) flowing one or more tethered oligonucleotides over the one or more patterns exposing the aldehyde-reactive substrate, wherein the one or more tethered oligonucleotides comprises a nucleotide sequence that, when hybridized to a complementary nucleotide sequence present in an untethered oligonucleotide, generates an enzyme cleavage site;
g) conjugating the 5′ amine-modified end of the one or more tethered oligonucleotides to the exposed aldehyde-reactive substrate within the one or more patterns; and
h) removing the cell-resistive layer.

26. The method of claim 25, wherein the method further comprises repeating steps b)-h) to create layers of the one or more patterns.

27. The method of claim 25, wherein the method further comprises, before step f), diluting the tethered oligonucleotides in a buffer.

28. The method of claim 27, wherein the method further comprises, before step g), incubating the one or more tethered oligonucleotides for about 5 minutes.

29. The method of claim 27, wherein the buffer is a sodium phosphate buffer.

30. The method of claim 29, wherein the concentration of the sodium phosphate buffer is about 50 mM.

31. The method of claim 28, wherein the method further comprises heating the solid support.

32. The method of claim 27, wherein the solid support is heated at 75° C. for about 60 minutes.

33. The method of claim 25, wherein said conjugating comprises performing amine-condensation comprising adding sodium dodecyl sulfate to the solid support.

34. The method of claim 33, wherein amine-condensation further comprises incubating the solid support at a temperature of from 90° C. to 100° C.

35. The method of claim 34, wherein the solid support is incubated for a period of time of from about 5 minutes to about 60 minutes.

36. The method of claim 34, wherein the incubation temperature is 75° C.

37. The method of claim 35, wherein the solid support is incubated for 30 minutes.

38. The method of claim 35, wherein the method further comprises rinsing the solid support with water.

39. The method of claim 25, wherein said conjugating comprises performing reductive-amination comprising adding sodium borohydride to the solid support.

40. The method of claim 39, wherein the method further comprises incubating the solid support for a period of time of from about 1 minute to about 30 minutes.

41. The method of claim 25, wherein removing the cell-resistive layer comprises:
i. rinsing the solid support; and r
ii. drying the solid support.

42. The method of claim 41, wherein said rinsing comprises rinsing the solid support with acetone.

43. The method of claim 41, wherein said drying comprises drying the solid support with nitrogen gas.

44. The method of claim 25, wherein the one or more tethered oligonucleotides are conjugated orthogonally relative to the aldehyde-reactive substrate.

45. The method of claim 25, wherein the target nucleotide sequence, when hybridized to a complementary nucleotide sequence present in an untethered oligonucleotide, generates a restriction enzyme cleavage site or a site that is cleavable by a CRISPR/Cas effector polypeptide complexed with a guide RNA.

46. The method of claim 25, further comprising flowing, onto the one or more patterns:
i) one or more cells;
ii) one or more effector molecules; or
iii) a combination of i) and ii),
wherein the one or more cells and the one or more effector molecules is bound to one or more untethered oligonucleotides comprising the complementary nucleotide sequence to the one or more tethered oligonucleotide sequences on the one or more patterns exposing the aldehyde-reactive substrate.

47. The method of claim 25, wherein the method comprises two or more tethered oligonucleotide sequences.

48. The method of any one of claims 25-47, wherein the cell-resistive layer is a photoresist layer.

49. The method of any one of claims 25-48, wherein the mask is a photomask.

50. The method of any one of claims 25-47, wherein the mask is an optical mask.

51. The method any one of claims 25-49, wherein the radiation is a beam of light.

52. The method of any one of claims 25-47, wherein the radiation is a beam of electrons.

53. The method of any one of claims 25-47, wherein the radiation is a beam of ions.

54. A solid support comprising:
a) one or more patterns exposing an aldehyde-reactive substrate;
b) one or more tethered oligonucleotides, covalently attached to the one or more patterns via an amine-modified termini at the 5′ end of the plurality of tethered oligonucleotides, wherein the one or more of tethered oligonucleotides comprises a nucleotide sequence that, when hybridized to a complementary nucleotide sequence present in an untethered oligonucleotide, generates a restriction enzyme cleavage site or a site that is cleavable by a CRISPR/Cas effector polypeptide;
c) one or more untethered oligonucleotides.
